(19) United States
(12) Patent Application Publication
(10) Pub. No.: US 2024/0225558 A1
(43) Pub. Date: Jul. 11, 2024

(54) INFORMATION PROCESSING APPARATUS AND INFORMATION PROCESSING METHOD

(71) Applicant: PARAMOUNT BED CO., LTD., Tokyo (JP)

(72) Inventors: Takashi ISHIKAWA, Tokyo (JP); Kazuki KOUKE, Tokyo (JP); Yuuki NAKAMURA, Tokyo (JP)

(73) Assignee: PARAMOUNT BED CO., LTD., Tokyo (JP)

(21) Appl. No.: 18/561,264

(22) PCT Filed: Jun. 28, 2022
(86) PCT No.: PCT/JP2022/025834

§ 371 Date: Nov. 15, 2023

(30) Foreign Application Priority Data
Dec. 7, 2021 (JP) 2021-198459

(51) Int. Cl. A61B 5/00; A61B 5/11; A61B 7/00; G06T 7/00; G06T 7/70; G16H 40/67

(52) U.S. Cl. CPC A61B 5/7275; A61B 5/0077; A61B 5/1117; A61B 5/4205; A61B 5/447; A61B 5/7267; A61B 7/008; G06T 7/0012; G06T 7/70; G16H 40/67; A61B 2505/07; G06T 2207/30004; G06T 2207/30196; G06T 2207/30232

(57) ABSTRACT

An information processing apparatus including: a receiver configured to acquire sensor information received from a wearable device and location information identifying a location of a communication device which receives the sensor information; and a controller configured to execute, based on the location information and the sensor information, an evaluation processing to evaluate a risk of a user who wears the wearable device, wherein the evaluation processing being configured to be changed based on the location information.

### TECHNICAL FIELD

The present invention relates to an information processing apparatus, an information processing method, and the like. This application claims priority from Japanese Patent Application No. 2021-198459, filed on Dec. 7, 2021, the contents of which are incorporated herein by reference.

### BACKGROUND

Heretofore, a system used in a scene where a caregiver provides assistance to a care recipient has been known. Patent Literature 1 discloses a method of disposing a sensor in a living space and generating provision information on a state of an inhabitant, living in the living space, based on time variation in detection information acquired by the sensor.

### CITATION LIST

### Patent Literature

PTL 1: Japan JPlaid-open application publication 2021-18760

### SUMMARY OF INVENTION

The present invention provides an information processing apparatus, an information processing method, and the like that appropriately support assistance provided to a care recipient by a caregiver.

An aspect of this disclosure relates to an information processing apparatus including: a receiver configured to acquire sensor information received from a wearable device and location information identifying a location of a communication device which receives the sensor information; and a controller configured to execute, based on the location information and the sensor information, an evaluation processing to evaluate a risk of a user who wears the wearable device, wherein the evaluation processing being configured to be changed based on the location information.

Another aspect of this disclosure relates to an information processing method including the steps of: acquiring sensor information received from a wearable device and location information identifying a location of a communication device which receives the sensor information; executing, based on the location information and the sensor information, an evaluation processing to evaluate a risk of a user who wears the wearable device, wherein the evaluation processing being configured to be changed based on the location information.

### BRIEF DESCRIPTION OF DRAWINGS

FIG. 1 is a diagram illustrating a configuration example of an information processing apparatus.

FIG. 2 is a diagram illustrating an example of arranging a wearable module and a communication device.

FIG. 3 is a diagram illustrating a configuration example of the wearable module.

FIG. 4 is a diagram illustrating a configuration example of the communication device.

FIG. 5 is a diagram illustrating a configuration example of an information processing system according to this embodiment.

FIG. 6 is a diagram illustrating a configuration example of a server system.

FIG. 7A is a diagram illustrating a display screen used in registration processing in the communication device.

FIG. 7B is a diagram illustrating a display screen used for pairing.

FIG. 7C is a diagram illustrating a display screen used for associating the wearable module and a care recipient with each other.

FIG. 8A is a diagram illustrating an example of a data structure of access point information.

FIG. 8B is a diagram illustrating an example of a data structure of module information.

FIG. 8C is a diagram illustrating an example of a data structure of notice management information.

FIG. 9 is a sequence diagram illustrating processing in the information processing system.

FIG. 10A illustrates an example of a screen showing a result of falling down determination processing.

FIG. 10B illustrates an example of the screen showing the result of the falling down determination processing.

FIG. 11 illustrates an example of the screen showing the result of the falling down determination processing.

FIG. 12 is a diagram illustrating sensor information corresponding to falling down from a bed.

FIG. 13 is a diagram illustrating sensor information corresponding to falling down from a wheelchair.

FIG. 14 is a diagram illustrating sensor information corresponding to falling down in a toilet.

FIG. 15 is a diagram illustrating sensor information corresponding to falling down during walking.

FIG. 16 is a diagram illustrating a configuration example of a neural network.

FIG. 17 is a diagram illustrating input data and output data in machine learning.

FIG. 18A is a diagram illustrating a pressure sensor disposed in the wheelchair.

FIG. 18B is a diagram illustrating a cross-sectional structure of a cushion disposed in the wheelchair.

FIG. 18C is a diagram illustrating a user interface unit and a notification unit provided in a control box.

FIG. 19A is a diagram illustrating a table which is a peripheral device.

FIG. 19B is a diagram illustrating a driving mechanism of the table.

FIG. 19C is a diagram illustrating a wheeled walker which is the peripheral device.

FIG. 19D is a diagram illustrating a driving mechanism of the wheeled walker.

FIG. 19E is a diagram illustrating the bed which is the peripheral device.

FIG. 20 is a diagram illustrating a configuration example of the peripheral device.

FIG. 21 is a diagram illustrating a configuration example of the information processing system according to this embodiment.

FIG. 22 is a sequence diagram illustrating processing in the information processing system.

FIG. 23 is a diagram illustrating implicit knowledge related to taking a meal.

FIG. 24 is a diagram illustrating devices arranged in a scene of taking a meal.

FIG. 25 is a diagram illustrating relationship among the devices, acquired data, and situations.

FIG. 26 is a diagram illustrating devices arranged in the vicinity of the bed.

FIG. 27 is a diagram illustrating labeled training data registered by a skilled worker.

FIG. 28 is a diagram illustrating labeled training data that is subjected to transparent processing and displayed at the time of assistance.

FIG. 29 illustrates an example of a display screen of a skeleton tracking result.

FIG. 30 is a diagram illustrating a device arranged in the vicinity of the wheelchair.

FIG. 31A illustrates an example of a display screen including a care recipient in an appropriate lateral position and the skeleton tracking result.

FIG. 31B illustrates an example of a display screen including a care recipient not in an appropriate lateral position and the skeleton tracking result.

FIG. 31C illustrates an example of a display screen including a care recipient in a supine position and the skeleton tracking result.

FIG. 32A illustrates an example of a display screen used for input data selection and the like in end-of-life care.

FIG. 32B illustrates an example of a display screen showing an analysis result in end-of-life care.

FIG. 32C illustrates an example of a display screen showing an analysis result in end-of-life care.

FIG. 32D illustrates an example of a display screen showing a detailed analysis result in end-of-life care.

FIG. 33 is a diagram illustrating devices operated in conjunction with an end-of-life care determination result.

FIG. 34A is a diagram illustrating a device and a scene in which recommendations are displayed.

FIG. 34B illustrates an example of a screen where recommendations are displayed.

FIG. 34C illustrates an example of a screen where recommendations are displayed.

### DETAILED DESCRIPTION OF THE INVENTION

Hereinbelow, this embodiment will be described with reference to the drawings. Throughout the drawings, the same or similar components are assigned with the same reference signs, and redundant description thereof will be omitted. Note that, this embodiment to be described below is not intended to unjustly limit the contents described in the scope of claims. In addition, not all configurations to be described in this embodiment are necessarily essential elements of this disclosure.

A method according to this embodiment is one in which, for work that a caregiver does according to his/her “feel” and “implicit knowledge” for example, such “feel” and “implicit knowledge” are digitized to give instructions to a caregiver so that the caregiver can provide appropriate assistance irrespective of his/her degree of proficiency. In addition, the method according to this embodiment is not limited to one for giving instructions to a caregiver, and may include one for directly controlling assistance instruments and the like. Hereinbelow, a specific method will be described.

Note that, the following mainly describes an example in which a caregiver is a nursing care staff of a nursing care facility and a care recipient is a user of the nursing care facility. For example, various devices such as a communication device 200 to be described later may be devices arranged in the nursing care facility. However, the method of this embodiment is not limited to this, and the caregiver may be a nurse or an assistant nurse of a hospital or may be a family member who provides nursing care at home to a person who needs nursing care. In addition, assistance in this embodiment may include help in actions such as taking a meal and voiding and personal care in daily life. For example, “assistance” in the following description may be replaced by “nursing care”.

### 1. System Configuration Example

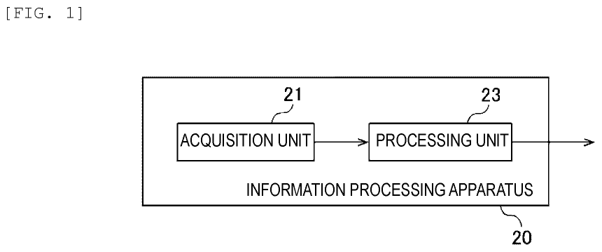

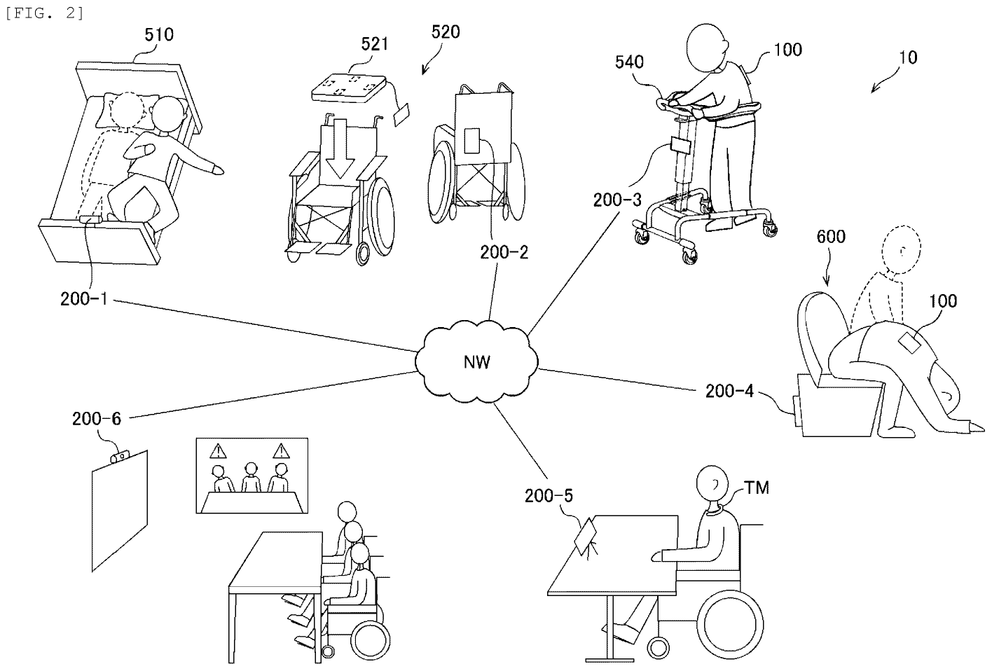

FIG. 1 is a diagram illustrating a configuration example of an information processing apparatus 20 of this embodiment. The information processing apparatus 20 includes an acquisition unit 21 (receiver) and a processing unit 23 (controller). However, the configuration of the information processing apparatus 20 is not limited to that of FIG. 1, and can be modified such as omitting a part of the configuration or adding a different configuration. For example, the information processing apparatus 20 may include units such as a storage unit, a display, and a user interface unit (not illustrated). In addition, the same goes for FIG. 2 and subsequent figures with regard to the point that the configuration can be modified such as omission or addition.

The acquisition unit 21 is configured to acquire information that associates sensor information, output from a wearable module 100 (wearable device), with location information identifying a location where the communication device 200 having received the sensor information is disposed. The wearable module 100 is a device that is worn by a care recipient to receive assistance, and the communication device 200 is a device that is disposed in a specific location. Note that, the wearable module 100 in this embodiment may be extended to a sensor module that moves along with the movement of a care recipient. For example, in the case of a sensor module for a care recipient who moves using a stick, a wheeled walker, a wheelchair, or the like, the sensor module may be mounted on the stick, the wheeled walker, the wheelchair, or the like. In addition, although a description will be provided in this embodiment using an example in which the wearable module 100 includes an acceleration sensor 120, the wearable module 100 is not limited to this and may include sensors such as a gyroscope sensor and a depth sensor, for example. In other words, although the following describes an example in which sensor information output from the wearable module 100 is information indicating acceleration, the sensor information may be other information such as angular speed and depth (distance). The wearable module 100 and the communication device 200 will be described later using FIGS. 2 to 4. Sensor information and location information will also be described in detail later.

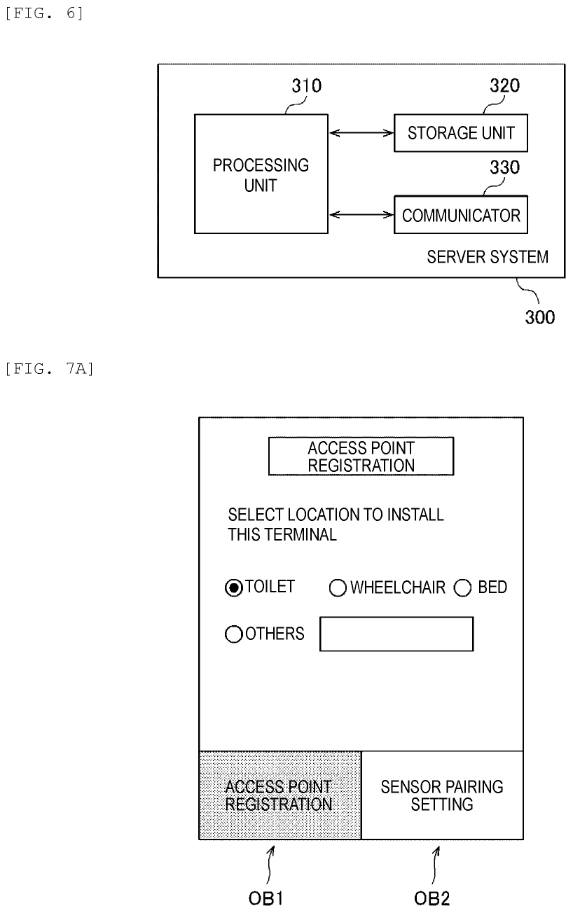

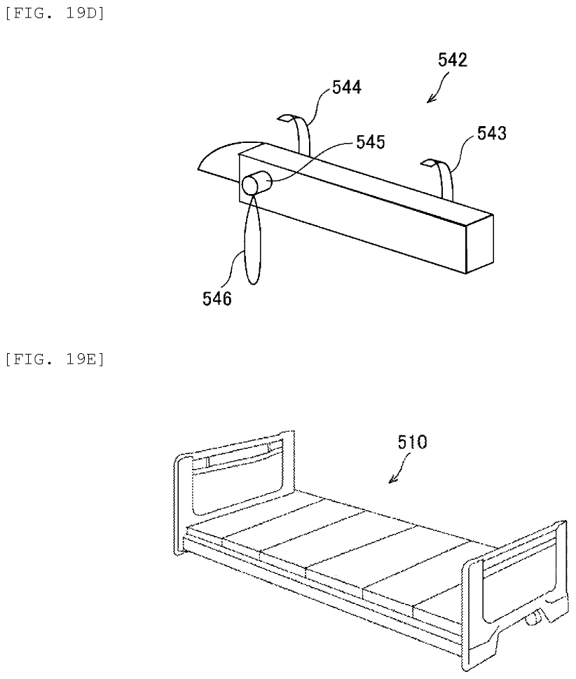

The processing unit 23 is configured to perform, based on location information and sensor information, intervention determination processing that is processing of determining as to whether intervention for a care recipient wearing the wearable module 100 is needed. The intervention mentioned here may be intervention by a caregiver, may be intervention using an assistance device, or may be both of them. Then, if determining that intervention is needed based on the intervention determination processing, the processing unit 23 causes various devices to perform intervention control that is control for causing them to intervene. The intervention control may be control for causing a caregiver terminal 400 to give notice prompting a caregiver to intervene. The caregiver terminal 400 is a device used by a caregiver who provides assistance to a care recipient. The caregiver terminal 400 will be described in detail later using FIG. 5. Alternatively, the intervention control may be control for operating a peripheral device 700 that is disposed in the vicinity of a care recipient and the communication device 200. Control over the peripheral device 700 will be described later using FIGS. 19A to 22.

For example, the processing unit 23 may execute, as the intervention determination processing described above, falling down determination processing based on location information and according to a location where the communication device 200 is disposed. Then, based on the falling down determination processing, the processing unit 23 causes the devices to perform intervention control that includes at least one of causing the caregiver terminal 400 to give notice of the risk of falling down and controlling the peripheral device 700. For example, the processing unit 23 may cause the caregiver terminal 400 and the peripheral device 700 to perform intervention control with detection of the risk of falling down as a trigger.

According to the method of this embodiment, in a case where the multiple communication devices 200 are arranged in a nursing care facility and the like, the location of a care recipient can be presumed according to which of the communication devices 200 has received sensor information. As a result, the intervention determination processing can be executed in consideration of the location, and thus processing precision can be improved. Since the location is identified automatically at this time, a caregiver does not need to perform a location setting operation, for example, so that the level of convenience can be increased. Hereinbelow, the method of this embodiment will be described in detail.

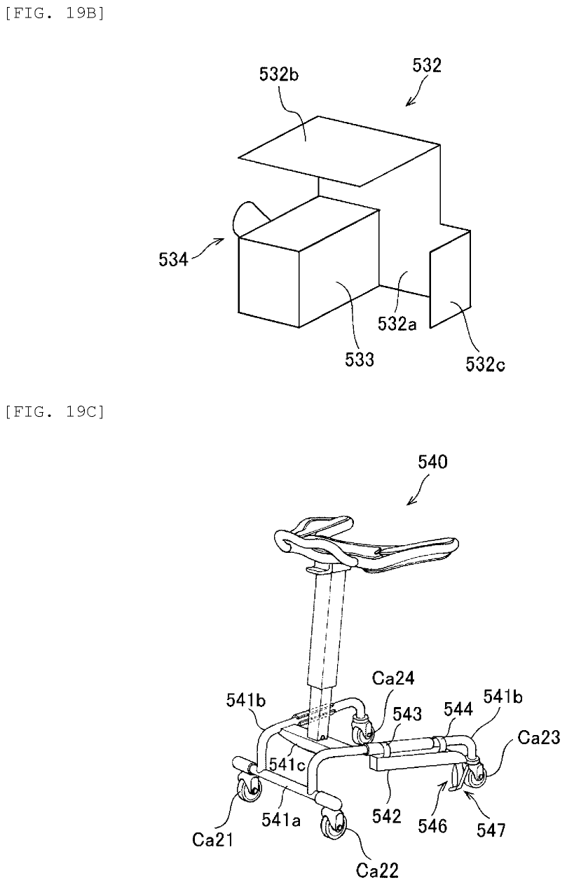

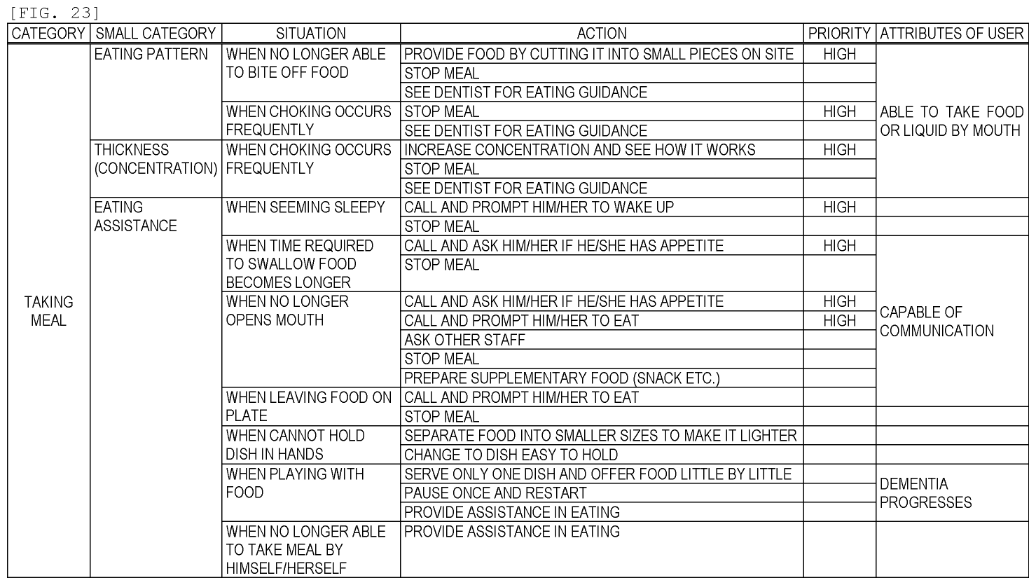

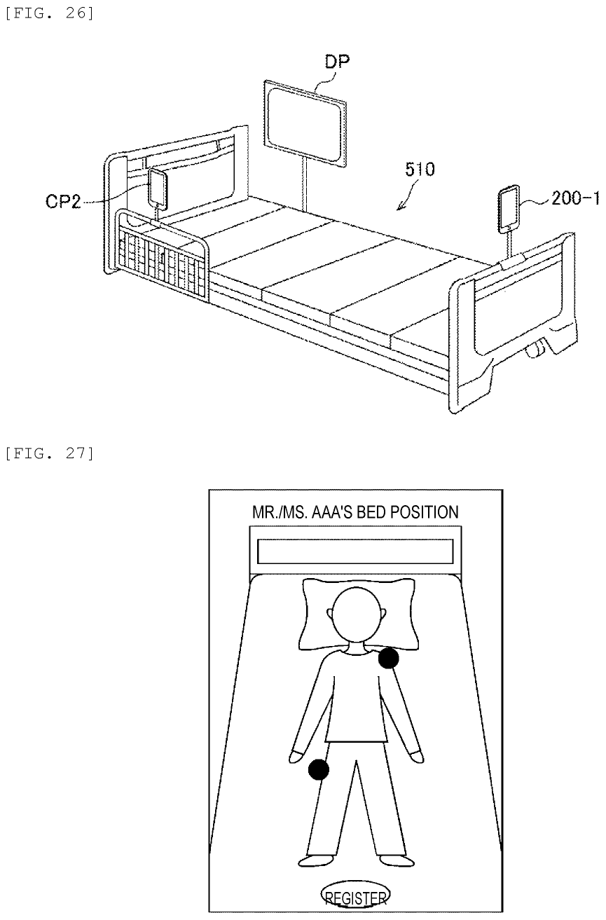

Note that, sensor for the information used intervention determination processing according to this embodiment is not limited to information output from the wearable module 100. For example, the acquisition unit 21 may acquire, as sensor information, information sensed by using at least one of a sensor in the communication device 200 and a sensor in a device disposed in the vicinity of the communication device 200. Since there is an increased degree of freedom in selecting a device that outputs sensor information, it is possible to acquire various kinds of sensor information and perform various kinds of the intervention determination processing. For example, as will be described later using FIGS. 18A to 18C, the contents of the falling down determination processing may be changed. In addition, while not limited to the falling down determination processing, the intervention determination processing may include determination in taking a meal which will be described later using FIGS. 23 to 25, determination in position adjustment which will be described later using FIGS. 26 to 29, and the like.

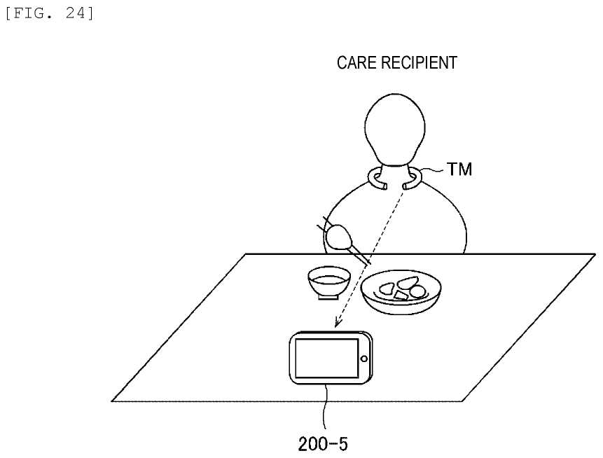

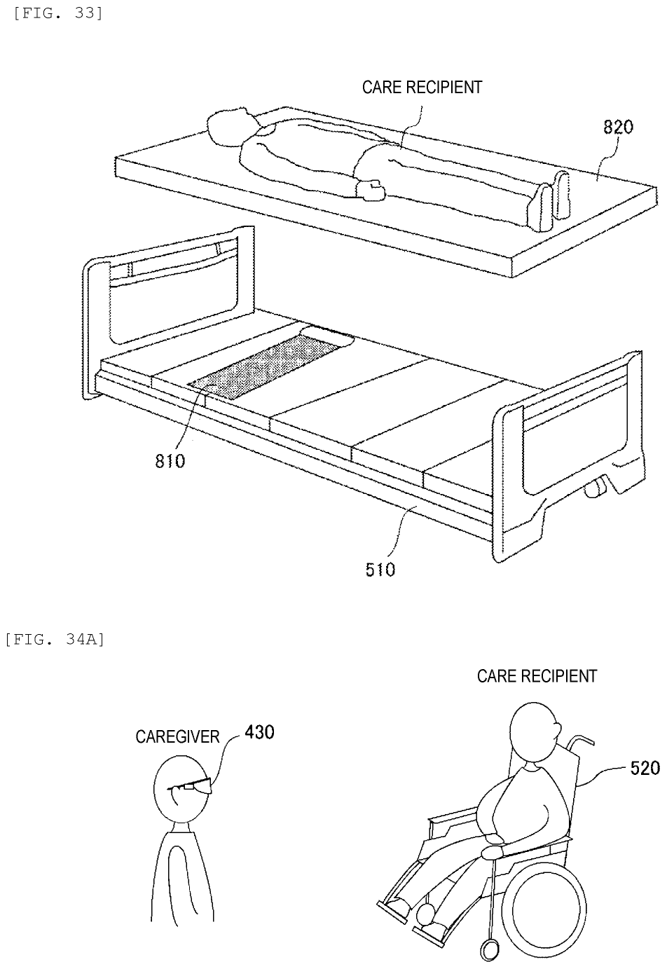

For example, the communication device 200 may include a camera, and sensor information may be an image taken by the camera. In addition, the device that outputs sensor information may be devices such as pressure sensors Se1 to Se4 which will be described later using FIG. 18A, a throat microphone TM which will be described later using FIG. 24, and a detection device 810 which will be described later using FIG. 33. In other words, the sensor information may be information indicating pressure, may be audio information, or may be information on heartbeat and respiration.

For example, as will be described later, sensor information used for the intervention determination processing may be switched depending on the location in such a way that the falling down determination processing using acceleration information from the wearable module 100 is performed in a toilet 600 and during walking and the falling down determination processing using pressure information from the pressure sensors Se1 to Se4 is performed during movement with a wheelchair 520. As can be understood from the above description, sensor information output from the wearable module 100 of this embodiment does not necessarily have to be used in all the locations and in all the intervention determination processing. To put it differently, a part of processes of the intervention determination processing may be processes not using sensor information from the wearable module 100.

FIG. 2 is a diagram illustrating a configuration example of an information processing system 10 of this embodiment, and specifically a diagram illustrating arrangement of the wearable module 100 and the communication device 200.

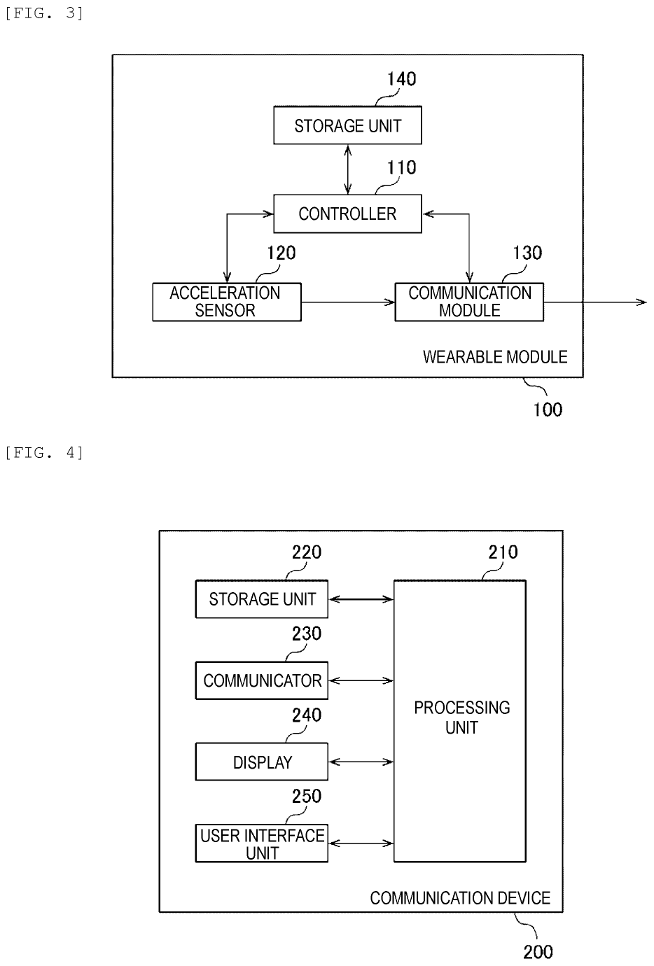

The wearable module 100 is a device worn by a care recipient to whom a caregiver provides assistance. The wearable module 100 is a plate-shaped device, for example, and may be fixed on the back of the care recipient or may be fixed on the chest thereof. The wearable module 100 may be attached on the clothes of the care recipient using a tape or the like. Alternatively, the wearable module 100 may be attached directly on the skin of the care recipient. Note that, the wearable module 100 is sufficient as long as it is a device worn by the care recipient, and a position at which the wearable module is fixed is not limited to the back or chest. A configuration example of the wearable module 100 will be described later using FIG. 3.

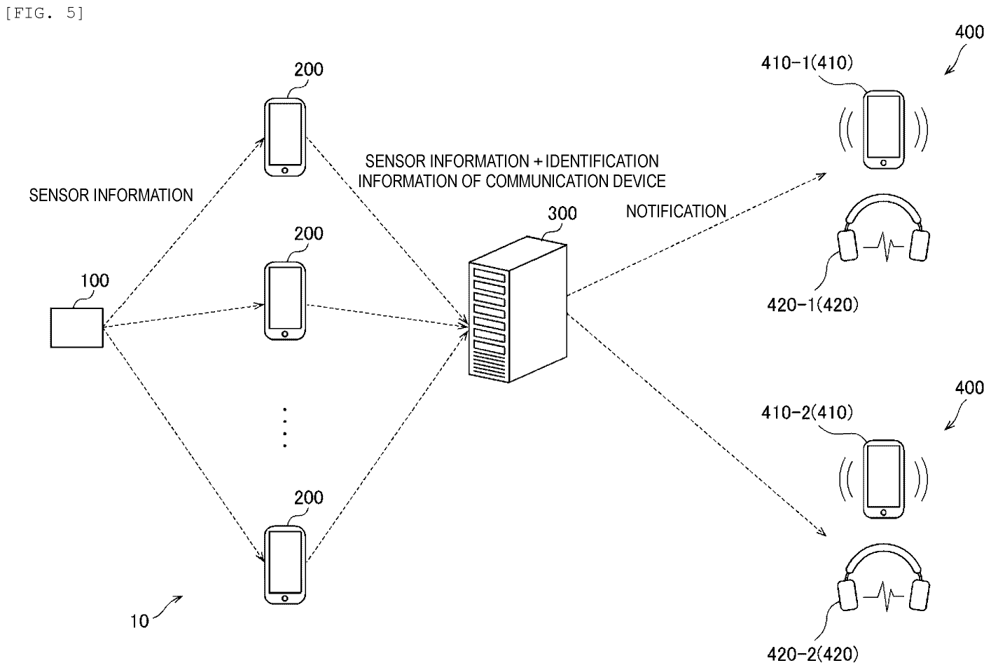

The communication device 200 is a device that performs communication with the wearable module 100. The communication device 200 may be communication equipment such as an access point or a router of a wireless Local Area Network (LAN), or may be a general-purpose terminal such as a smartphone. A configuration example of the communication device 200 will be described later using FIG. 4.

The number of the communication devices 200 in this embodiment may be two or more. Although FIG. 2 illustrates six communication devices 200-1 to 200-6 as the communication devices 200, the number of the communication devices 200 is not limited to this. The multiple communication devices 200 may be respectively arranged at different locations. The locations where the communication devices 200 are arranged include a bed 510, the wheelchair 520, a wheeled walker 540, the toilet 600, a dining room, a living room, and the like. Each of the communication devices 200-1 to 200-6 is connected to a network NW. The network NW may be a public communication network such as the Internet, or may be an internal network such as an intranet in the nursing care facility.

In the example of FIG. 2, the communication device 200-1 is disposed in the bed 510 used by a care recipient for sleeping and the like. For example, a holder of any shape (e.g. a holder formed by providing a rectangular cutout in a foot board on the bed's inner side) is attached to a part of the bed 510, and the communication device 200-1 is held by the holder. Here, the bed 510 is a mobile bed capable of automatically changing the angle and height of sections, for example, but a bed without such a function may be used instead. Note that, the sections are surfaces on which to place a mattress and the like, and may have any shape such as a plate shape and a mesh shape. In addition, the communication device 200-1 is sufficient as long as it can be associated with the bed 510, and may be disposed, for example, at a location such as a wall surface or a floor surface of a room where the bed 510 is disposed or a furniture other than the bed 510. Further, as will be described later using FIG. 26, other devices may be arranged in the vicinity of the bed 510.

The communication device 200-2 and the communication device 200-3 are arranged in devices used for assistance in movement of a care recipient. The communication device 200-2 is disposed in the wheelchair 520. For example, a pocket is provided on a back surface of the wheelchair 520, and the communication device 200-2 is put into the pocket. In addition, a cushion 521 disposed in the wheelchair 520 may be provided with the pressure sensors Se1 to Se4. The pressure sensors Se1 to Se4 will be described later using FIG. 18A. The communication device 200-3 is disposed in the wheeled walker 540 used by a care recipient for moving. The communication device 200-3 is disposed at a support of the wheeled walker 540, for example.

The communication device 200-4 is disposed in the toilet 600 used by a care recipient. The communication device 200-4 may be disposed at a tank or the like of the toilet 600, or may be disposed at a floor surface or a wall surface.

The communication device 200-5 and the communication device 200-6 are arranged at locations where a care recipient acts away from his/her room. The communication device 200-5 is disposed in the dining room. For example, as illustrated in FIG. 2, the communication device 200-5 may be disposed on a table of the dining room at a position facing a care recipient in the middle of a meal. In addition, in the middle of a meal, the throat microphone TM that detects swallowing and choking may be used, for example. The devices used in the middle of a meal will be described in detail later using FIG. 24. The communication device 200-6 is disposed in a location, such as a living room or a hall, where many people can do activities. For example, as illustrated in FIG. 2, the communication device 200-6 may be fixed at a location such as a TV set disposed in the living room.

In addition, another communication device 200 not illustrated in FIG. 2 may be disposed in another location of the nursing care facility and the like. For example, the communication device 200 may be disposed in the nursing care facility at a location where a care recipient walks. Various modifications, such as a corridor and a stair, are possible as the location where the communication device 200 is disposed. Such a communication device 200 is used for assisting in walking of a care recipient who can walk on his/her own, for example.

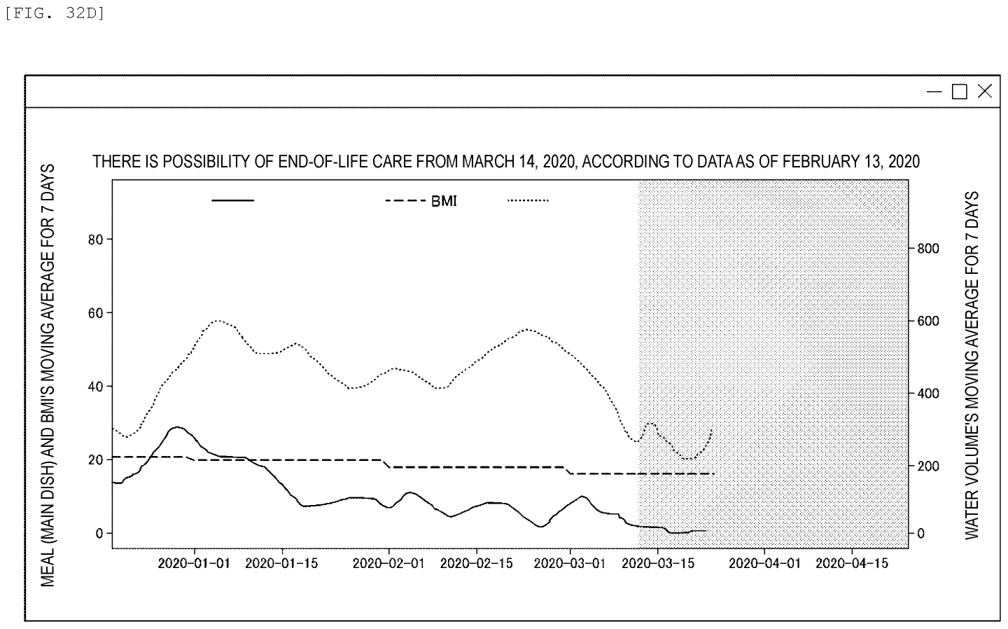

In addition, as will be described later, the intervention determination processing according to this embodiment may include end-of-life-care related processing. On the basis of the end-of-life-care related processing result, display of screens to be described later using FIGS. 32A to 32D, change of processing mode based on an output from the detection device 810, and the like are executed. The end-of-life-care related processing may be executed in conjunction with the intervention determination processing at each location illustrated in FIG. 2. For example, algorithms and parameters (such as thresholds) used for the end-of-life-care related processing may be changed based on the intervention determination processing at each location. Alternatively, algorithms and parameters used for the processing at each location may be changed based on the end-of-life-care related processing. The end-of-life-care related processing will be described in detail later.

Communication between the communication device 200 and the wearable module 100 may be communication using Bluetooth (registered trademark), may be communication using wireless LAN defined in IEEE802.11, or may be communication using other methods.

The communication device 200 may be a device that receives, as an access point, communication connection from the wearable module 100. Here, the access point indicates a device that directly receives sensor information from the wearable module 100. Note that, to directly receive sensor information specifically means to receive sensor information without via other communication devices 200. For example, consider a case where the wearable module 100 establishes connection with the communication device 200-1 using Bluetooth or the like and transmits sensor information to the communication device 200-1 using this connection, and then the communication device 200-1 transfers the sensor information to the communication device 200-2. In this example, the communication device 200-1 serves as an access point for the wearable module 100, but the communication device 200-2 does not serve as an access point.

For example, the communication device 200 may serve as central in Bluetooth, and the wearable module 100 may serve as peripheral in Bluetooth. Alternatively, the communication device 200 may serve as an access point (AP) in wireless LAN, and the wearable module 100 may serve as a station (STA) in wireless LAN. As can be understood from the above example, the access point in this embodiment is not limited to an AP in wireless LAN, and widely includes devices that directly performs communication with the wearable module 100 using other communication methods.

The communication device 200 with which the wearable module 100 performs communication may vary depending on the position of the wearable module. For example, the position of the wearable module 100 changes along with the movement of a care recipient who wears this wearable module 100. When the communication device 200 exists within predetermined distance or smaller, the wearable module 100 tries to get connected to this communication device 200. The predetermined distance mentioned here may be a distance within which Bluetooth advertising packets can be transmitted and received, may be a distance within which Service Set Identifier (SSID) broadcasting signals in wireless LAN can be transmitted and received, or may be a distance defined by other communication methods.

Alternatively, to be located at a position sufficiently close to the communication device 200 may be used as a connection condition. For example, connection between the wearable module 100 and the communication device 200 may be established on condition that the intensity of received radio wave in transmission/reception of advertising packets and SSID broadcasting signals is equal to or larger than a given threshold. In addition, if multiple communication devices 200 are detected within a predetermined distance range from the wearable module 100, the wearable module 100 may select the communication device 200, with which it establishes connection, based on the intensity of received radio wave.

FIG. 3 is a diagram illustrating a configuration example of the wearable module 100. The wearable module 100 includes: a controller 110; the acceleration sensor 120; a communication module 130; and a storage unit 140. In addition, the wearable module 100 may also include a configuration (not illustrated) such as a temperature sensor.

The controller 110 is configured to perform control over various parts of the wearable module 100 such as the acceleration sensor 120 and the communication module 130. The controller 110 may be a processor. For the processor mentioned here, various processors such as a Central Processing Unit (CPU), a Graphics Processing Unit (GPU), and a Digital Signal processor (DSP) can be used.

The acceleration sensor 120 is a sensor that detects acceleration and outputs sensor information which is a detection result. For example, the acceleration sensor 120 may be a 3-axis acceleration sensor that detects 3-axis translational acceleration. The sensor information in this case indicates a set of acceleration values in each of the x, y, and z axes. For example, in a state where the wearable module 100 is mounted on the chest of a care recipient, the x axis may be an axis corresponding to a front-rear direction of the care recipient, the y axis may be an axis corresponding to a left-right direction thereof, and the z axis may be an axis corresponding to a vertically up-down direction thereof. Note that, the acceleration sensor 120 may alternatively be a 6-axis acceleration sensor that detects 3-axis translational acceleration and angular acceleration around each axis, and its specific aspects can be modified in various ways.

The communication module 130 is an interface for performing communication via a network, and includes an antenna, a radio frequency (RF) circuit, and a baseband circuit, for example. The communication module 130 may be operated under control of the controller 110 or may include a processor for communication control that is different from the controller 110. As described previously, the communication module 130 may perform communication using wireless LAN, may perform communication using Bluetooth, or may perform communication using other methods.

The communication module 130 is configured to transmit, to the communication device 200, sensor information output from the acceleration sensor 120. As described previously, when the communication device 200 exists within a predetermined distance, for example, the communication module 130 establishes connection with this communication device 200 and transmits sensor information to the communication device 200 with which the communication module has established connection.

The storage unit 140 is a work area of the controller 110, and is implemented by various memories such as SRAM, DRAM, and Read Only Memory (ROM). The storage unit 140 may store sensor information acquired by the acceleration sensor 120. For example, if the communication module 130 fails to transmit sensor information to the communication device 200, the storage unit 140 may store the sensor information not transmitted. In this case, the storage unit 140 may store, together with the sensor information, the reason why the sensor information has failed to be transmitted to the communication device 200 and error contents. If communication with the communication device 200 becomes available, the communication module 130 transmits the sensor information accumulated in the storage unit 140 to the communication device 200. The communication module 130 may also transmit the reason why transmission has failed and the error contents described above while associating them with the sensor information.

FIG. 4 is a diagram illustrating a configuration example of the communication device 200. For example, the communication device 200 includes: a processing unit 210; a storage unit 220; a communicator 230; a display 240; and a user interface unit 250.

The processing unit 210 includes the following hardware. The hardware can include at least one of a circuit that processes a digital signal and a circuit that processes an analog signal. For example, the hardware may include one or more circuit devices and one or more circuit elements mounted on a circuit board. Examples of the one or more circuit devices are an Integrated Circuit (IC), a field-programmable gate array (FPGA), and the like. Examples of the one or more circuit elements are a resistor, a capacitor, and the like.

Alternatively, the processing unit 210 may be implemented by the following processor. The communication device 200 of this embodiment includes: a memory that stores information; and a processor that operates based on the information stored in the memory. For example, the information is a program, various kinds of data, and the like. The processor includes hardware. Various processors such as a CPU, a GPU, and a DSP can be used for the processor. The memory may be a semiconductor memory such as a Static Random Access Memory (SRAM), a Dynamic Random Access Memory (DRAM), and a flash memory, may be a register, may be a magnetic memory device such as a Hard Disk Drive (HDD), or may be an optical memory device such as an optical disk device. For example, the memory stores a computer readable command, and the function of the processing unit 210 is implemented as processing by causing the processor to execute the command. The command mentioned here may be a command of a command set constituting a program or may be a command that gives operation instructions to a hardware circuit of the processor.

The storage unit 220 is a work area of the processing unit 210, and is implemented by various memories such as SRAM, DRAM, and ROM.

The communicator 230 is an interface for performing communication via a network, and includes an antenna, an RF circuit, and a baseband circuit, for example. For example, the communicator 230 performs first communication with the wearable module 100 and performs second communication with a server system 300 which will be described later using FIG. 5.

Note that, the communication methods of the first communication and the second communication may be the same or different from each other. For example, in a case where the communication methods of the first communication and the second communication are the same, the communicator 230 may include one wireless communication chip and use this wireless communication chip in a time division manner, or may include two wireless communication chips of the same communication method. Meanwhile, in a case where the communication methods of the first communication and the second communication are different from each other, the communicator 230 may include two wireless communication chips of different communication methods. The first communication may be communication using Bluetooth or may be communication using wireless LAN, as described above. The second communication may be communication using wireless LAN or may be communication using a mobile communication network such as Long Term Evolution (LTE) or 5G.

Note that, the first communication using Bluetooth may be performed by a beacon method or by a connection method. The beacon method is a method in which data is transmitted for every predetermined period of time (e.g. one minute), and the connection method is a method which uses a user operation as a trigger for data transmission. The user operation is an operation of pressing an update button, for example. The update button may be provided in the wearable module 100 or may be displayed on the display 240 of the communication device 200. Alternatively, the update button may be displayed on a display or the like of a device other than the communication device 200, such as the caregiver terminal 400 to be described later, and when this operation is performed, the fact that the operation has been performed may be transmitted to the wearable module 100 and the communication device 200. In this manner, multiple methods having different data transmission/reception timings may be used in the first communication. The same goes for the case of using a method other than Bluetooth as the first communication. In addition, multiple methods having different data transmission/reception timings may also be used in the second communication.

The display 240 is an interface for displaying various kinds of information, and may be a liquid crystal display, an organic EL display, or a display of other methods.

The user interface unit 250 is an interface for receiving the user operation. The user interface unit 250 may be a button and the like provided in the communication device 200. In addition, the display 240 and the user interface unit 250 may be formed in one unit as a touch panel.

The communication device 200 may also include a configuration not illustrated in FIG. 4 such as a light emitting unit, a vibration unit, and a sound output unit. The light emitting unit is a light emitting diode (LED) for example, and is configured to give notification by emission of light. The vibration unit is a motor for example, and is configured to give notification by vibration. The sound output unit is a speaker for example, and is configured to give notification by sound. The communication device 200 may also include various sensors including a motion sensor such as an acceleration sensor and a gyroscope sensor, an imaging sensor, and a Global Positioning System (GPS) sensor.

By using the information processing system 10 illustrated in FIG. 2, it is possible to presume the situation of a care recipient according to which communication device 200 the wearable module 100 is connected to or, in a more limited sense, according to which communication device 200 sensor information of the wearable module 100 is transmitted to.

For example, if sensor information is transmitted to the communication device 200-1, it is presumed that a care recipient is lying on the bed 510 or sitting on the bed 510. If the sensor information is transmitted to the communication device 200-2 or the communication device 200-3, it is presumed that the care recipient is moving using the wheelchair 520 or the wheeled walker 540. If the sensor information is transmitted to the communication device 200-4, it is presumed that the care recipient is in the toilet.

If the sensor information is transmitted to the communication device 200-5 or the communication device 200-6, it is presumed that the care recipient is doing activities in the corresponding location such as the dining room or the living room.

Once the situation has been presumed, assistance to be provided can be presumed. For example, in a case where the care recipient is located near the bed 510, assistance to be provided includes assistance such as patrols while asleep, changing of diapers, position adjustment for preventing bed sore, and assistance in movement to the wheelchair 520. Meanwhile, in a case where the care recipient is riding on the wheelchair 520, assistance to be provided includes assistance in movement using the wheelchair 520 and meal assistance. In a case where the care recipient is in the toilet, assistance in voiding in the toilet is provided. In a case where the care recipient is walking, assistance in prevention of falling down and the like is provided.

As a result, it is possible to identify implicit knowledge for appropriately executing assistance presumed and notify a caregiver of specific actions for using the implicit knowledge, for example. For example, in the case of performing the falling down determination processing using the acceleration sensor 120 of the wearable module 100, it is possible to execute determination using criteria different depending on the location. Meanwhile, in the case of performing the intervention determination processing using sensor information from the communication device 200 and other devices, control to activate sensors in the communication device 200 and the devices may be performed. This makes it possible to acquire sensor information appropriate depending on the location, and thus possible to improve determination precision. Note that, control to activate/deactivate the sensors does not necessarily have to be performed automatically based on the connection status between the wearable module 100 and the communication device 200, and a part of or all the sensors may be activated manually. In this way, according to the method of this embodiment, it is possible to automatically presume the location of a care recipient, and thus possible to support assistance according to the situation without manually setting the specific situation.

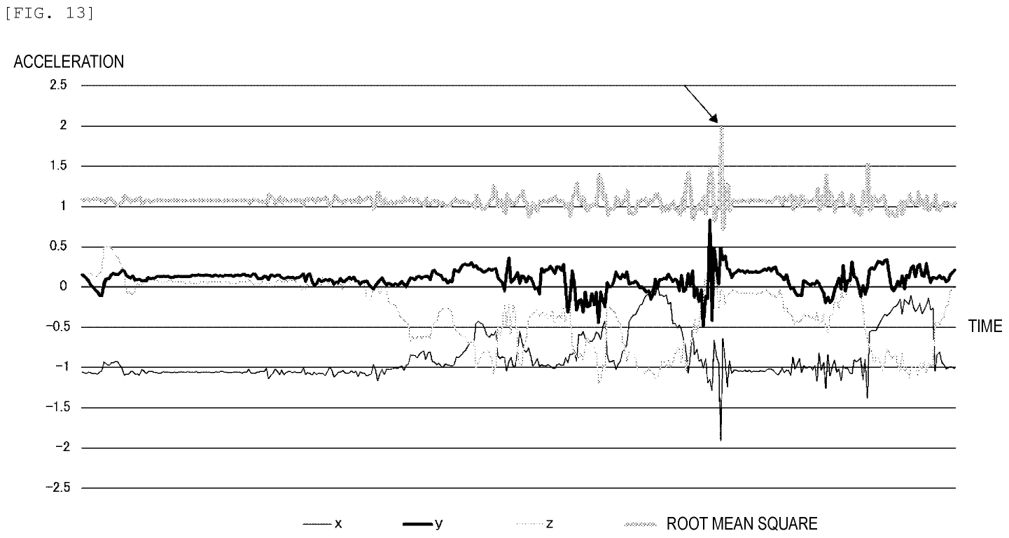

For example, a nursing care staff of a nursing care facility needs to provide the various kinds of nursing care described above to a lot of care recipients, and therefore performs tasks according to a very tight schedule. In addition, if an irregular event such as leakage of stools or falling down occurs, the original schedule is difficult to complete, and hence a nursing care staff sometimes needs to deal with this by leaving a part of assistance until later according to the order of priority, for example. For this reason, even if a system for supporting a caregiver is provided and this system has a configuration in which support contents are customizable according to the situation, a nursing care staff has no room to customize the contents point by point. For example, as will be described later using FIGS. 12 to 15, in processing of supporting a caregiver by showing the risk of falling down, processing precision is improved by setting individual determination thresholds and the like for falling down from the bed 510, falling down from the wheelchair 520, falling down in the toilet 600, and falling down during walking, respectively. However, to make a nursing care staff do such individual settings is not preferable in terms of user-friendliness.

In that respect, according to the method of this embodiment, since the setting can be automated based on the communication status between the wearable module 100 and the communication device 200, it is possible to appropriately support assistance by a caregiver while suppressing an increase in the burden on a caregiver and the like.

FIG. 5 is a diagram illustrating a specific configuration example of the information processing system 10 according to this embodiment. The information processing system 10 may include the server system 300 and the caregiver terminal 400 in addition to the wearable module 100 and the communication device 200 illustrated in FIG. 2. Note that, the example illustrated here is an example of giving notification to the caregiver terminal 400 as a result of the intervention determination processing.

The server system 300 is configured to perform communication with the communication device 200 via the network NW illustrated in FIG. 2, for example. The network NW mentioned here may be a public communication network such as the Internet. In this case, information from the wearable module 100 collected by the communication device 200 is subject to processing using the cloud. Alternatively, the network NW may be an internal network such as an intranet of a nursing care facility. The server system 300 in this case is a management server provided inside the nursing care facility, for example.

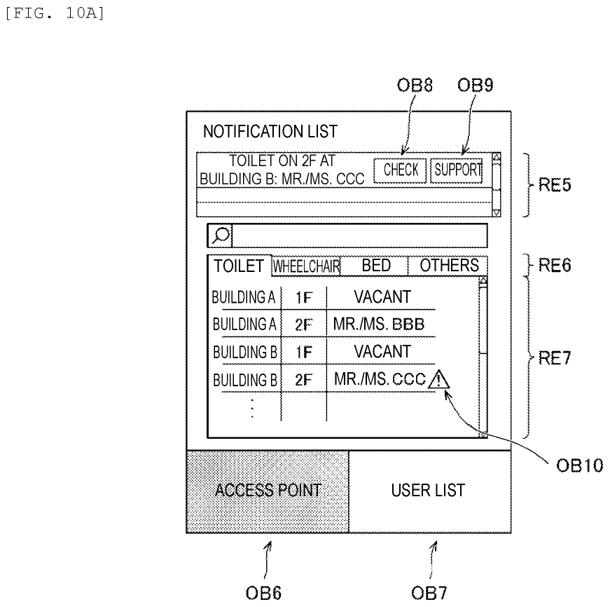

The server system 300 may be one server or may include multiple servers. For example, the server system 300 may include a database server and an application server. The database server is configured to store various kinds of data such as data transmitted by the communication device 200 and processing algorithms. The application server corresponds to a processing unit 310 which will be described later, and performs processing such as Steps S106 to S108 of FIG. 9. Note that, the multiple servers mentioned here may be physical servers or may be virtual servers. In addition, in the case of using virtual servers, the virtual servers may be provided in one physical server or may be dispersed in multiple physical servers. As described above, the specific configuration of the server system 300 of this embodiment can be modified in various ways.

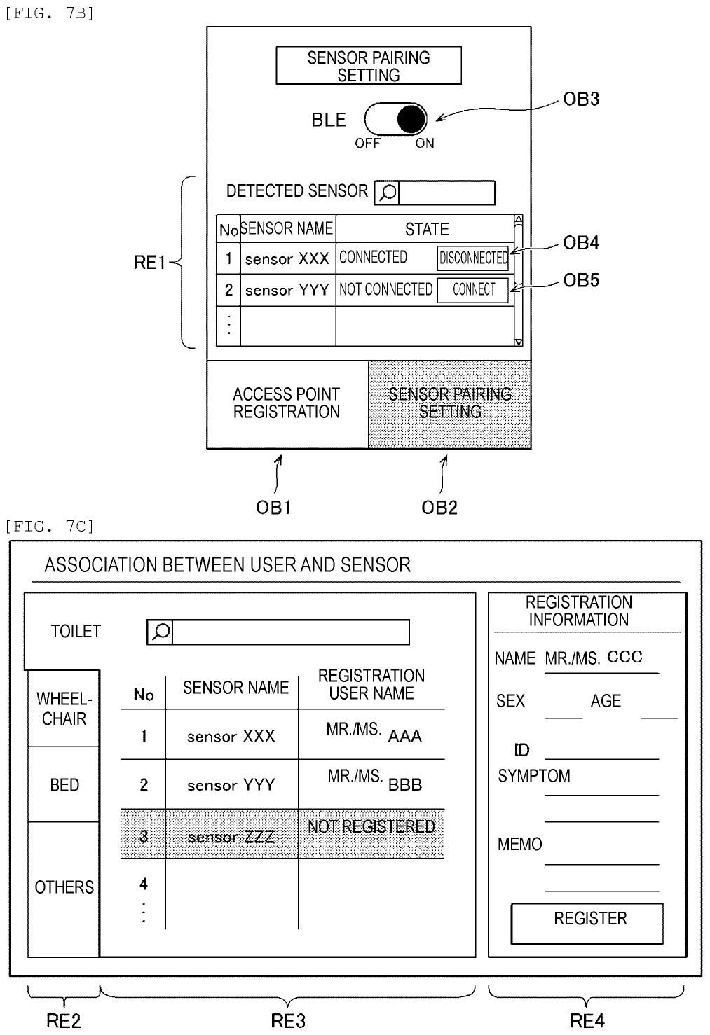

FIG. 6 is a diagram illustrating a configuration example of the server system 300. For example, the server system 300 includes: the processing unit 310; a storage unit 320; and a communicator 330.

The processing unit 310 includes hardware including at least one of a circuit that processes a digital signal and a circuit that processes an analog signal. For example, the hardware may include one or more circuit devices and one or more circuit elements mounted on a circuit board.

Alternatively, the processing unit 310 may be implemented by a processor including the hardware. The server system 300 includes a processor and a memory. Various processors such as a CPU, a GPU, and a DSP can be used for the processor. The memory may be a semiconductor memory, may be a register, may be a magnetic memory device, or may be an optical memory device. For example, the memory stores a computer readable command, and the function of the processing unit 310 is implemented as processing by causing the processor to execute the command.

The storage unit 320 is a work area of the processing unit 310, and is implemented by various memories such as SRAM, DRAM, and ROM.

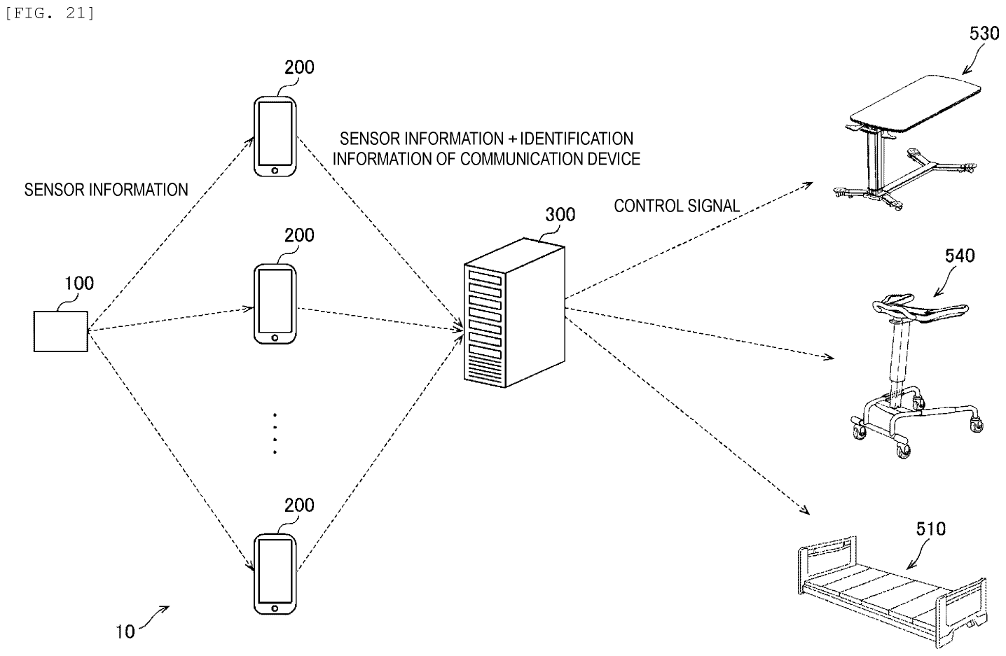

The communicator 330 is an interface for performing communication via a network, and includes an antenna, an RF circuit, and a baseband circuit, for example. The communicator 330 is configured to perform communication with the communication device 200 and the caregiver terminal 400, for example. In addition, as will be described later using FIG. 21, the communicator 330 may perform communication with a peripheral device 700. Note that, at least one of communication with and the caregiver terminal 400 communication with the peripheral device 700 may be executed via the communication device 200, and their specific connection aspects can be modified in various ways.

The caregiver terminal 400 is a device used by a caregiver at a location such as a nursing care facility, and is a device used to present information to a caregiver or accept input of information by a caregiver. For example, the caregiver terminal 400 may be a device carried or worn by a caregiver.

For example, as illustrated in FIG. 5, the caregiver terminal 400 may include: a mobile terminal device 410; and a headset 420. The mobile terminal device 410 is a smartphone, for example, but may be other portable devices. The headset 420 is a device wearable by a caregiver, and includes an earphone or a headphone and a microphone. In addition, the headset 420 may be changed to other wearable devices such as a glasses-type device or a watch-type device. Note that, the glasses-type device may include an Augmented Reality (AR) glass and a Mixed Reality (MR) glass. In addition, the caregiver terminal 400 may be other devices such as a Personal Computer (PC).

Note that, FIG. 5 illustrates two sets of the caregiver terminals 400 that each include the mobile terminal device 410 (the mobile terminal devices 410-1 and 410-2) and the headset 420 (the headsets 420-1 and 420-2). Note that, the number of the caregiver terminals 400 is not limited to two. In addition, the types and the number of devices constituting each caregiver terminal 400 are not limited to those in the example of FIG. 5, and can be modified in various ways.

An operation example of the information processing system 10 will be described. As described previously, the wearable module 100 transmits sensor information to any of the communication devices 200. The communication device 200 associates the received sensor information with location information identifying a location where this communication device 200 is disposed. The location information mentioned here is information enabling identification of the location where the communication device 200 is disposed, and may be flag information or may be identification information of the communication device 200.

The flag information is 4-bit data each indicating any of the toilet 600, the bed 510, the wheelchair 520, and walking, for example, and is data in which one bit value is 1 and the remaining three bit values are 0. However, the data format of the flag information is not limited to this, and the flag information may be 2-bit data that uses four values of 00, 01, 10, and 11 to distinguish between the four types of data, i.e., the toilet 600, the bed 510, the wheelchair 520, and walking, or may be data of other formats. In addition, the location where the communication device 200 is disposed is not limited to four, and thus the flag information may be data including more bits.

In a case where the communication device 200 is a smartphone, for example, the identification information of the communication device 200 is information on Subscriber Identity Module (SIM). However, the identification information may be any information as long as it uniquely identifies the communication device 200, and other information such as a MAC address and a serial number may be used as the identification information.

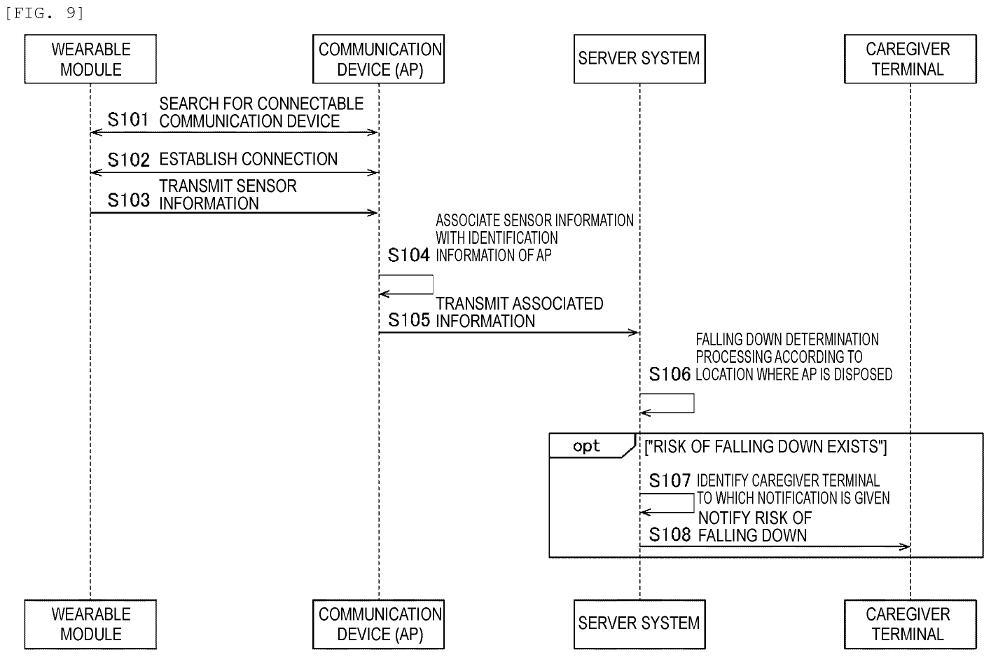

As will be described later using FIG. 8A, in the method of this embodiment, access point information that associates the identification information of the communication device 200 with the flag information identifying the location may be stored. In this case, the flag information can be identified based on the identification information of the communication device 200. In other words, the flag information and the identification information of the communication device 200 are information enabling identification of the location of the communication device 200, and are included in the location information of this embodiment. For example, the communication device 200 may associate sensor information with flag information. Alternatively, the communication device 200 may associate sensor information with the identification information of this communication device 200 and the server system 300 may perform processing of identifying flag information based on the identification information. Hereinbelow, an example of the latter case will be described.

The processing unit 310 of the server system 300 finds, based on the identification information and the sensor information, information that supports assistance provided to a care recipient who wears the wearable module 100. Specifically, the processing unit 310 makes determination based on the implicit knowledge of a skilled worker, and outputs information that enables a caregiver to deal with things as a skilled worker does even if the degree of proficiency of the caregiver is low. As an example, the sensor information is acceleration information, and the processing unit 310 may perform the falling down determination processing of determining the risk of falling down of a care recipient. Note that, the falling down determination processing mentioned here is sufficient as long as it includes processing for determining whether the risk of falling down exists and the level of the risk, and is not limited to processing of detecting an event of falling down itself. The falling down determination processing of this embodiment may include processing of determining a state in a previous phase of falling down, such as posture determination processing of determining whether a care recipient is off balance and his/her posture is likely to cause falling down. In this event, since this embodiment can implement the falling down determination processing according to the location where the communication device 200 is disposed based on the identification information of the communication device 200, it is possible to improve precision. In the example of FIG. 2, the falling down determination processing according to the location includes: determination of falling down from the bed 510; determination of falling down from the wheelchair 520; determination of falling down in the toilet 600; and determination of falling down during walking. The processing will be described in detail later.

Then, if the risk of falling down is detected based on the falling down determination processing, the processing unit 310 gives notification of the risk of falling down to the caregiver terminal 400 via the communicator 330. The specific notification will be described later.

The information processing apparatus 20 described above using FIG. 1 corresponds to the server system 300, for example. In other words, the acquisition unit 21 that acquires sensor information and location information while associating them with each other may be an interface through which to acquire data (such as the communicator 330). In addition, the processing unit 23 of the information processing apparatus 20 may be the processing unit 310 illustrated in FIG. 6.

In a case where the server system 300 is a device provided in an external network of a nursing care facility, for example, information that associates sensor information and location information with each other can be managed using the cloud. For example, by integrating pieces of information of multiple nursing care facilities, improvement in processing precision and the like can be easily achieved. In addition, since the communication device 200 does not need to execute the falling down determination processing, it is possible to decrease processing load on the communication device 200. Meanwhile, even in a case where the server system 300 is a management server or the like provided in an internal network of a nursing care facility, since processing can be aggregated in this management server, processing load on the communication device 200 can be decreased in the same way.

However, the information processing apparatus 20 of this embodiment is not limited to the server system 300. For example, the information processing apparatus 20 may be the communication device 200. The processing unit 210 of the communication device 200 may include: an association processing unit that associates sensor information, acquired from the wearable module 100, with the identification information of the communication device 200 itself or flag information; and a falling down determination processing unit that performs the falling down determination processing based on the information thus associated. In this case, the acquisition unit 21 of the information processing apparatus 20 may serve as the association processing unit and the processing unit 23 of the information processing apparatus 20 may serve as the falling down determination processing unit.

This enables the communication device 200 to execute processing using sensor information. In this case, the server system 300 can be omitted. As a result, the closed information processing system 10 can be constructed in a nursing care facility while not using the external cloud, for example, which facilitates system construction and suppresses security risk such as data leakage. Or alternatively, no dedicated management server needs to be provided in a nursing care facility, which facilitates system construction.

For example, the communication device 200 according to this embodiment may be a smartphone. In this case, both the communication device 200 and the caregiver terminal 400 can be implemented by a smartphone. In other words, the necessity of introducing a dedicated device for constructing the information processing system 10 of this embodiment becomes low. For example, even in a case where there is no Wi-Fi (registered trademark) environment in a nursing care facility, the method of this embodiment can be employed easily.

Note that, in a case where the communication device 200 serves as the information processing apparatus 20, this information processing apparatus 20 is not limited to the communication device 200 that directly acquires sensor information. For example, after receiving sensor information from the wearable module 100 and associating location information with this sensor information, the communication device 200-1 may transmit the associated information to another communication device 200 such as the communication device 200-2. Then, the processing unit 210 of the communication device 200-2 may perform the falling down determination processing based on the information that associates the location information and the sensor information with each other.

In this case, the information processing apparatus 20 may be the communication device 200-2, and the acquisition unit 21 of the information processing apparatus 20 may be an interface through which to transmit and receive data with the communication device 200-1 (such as the communicator 230 of the communication device 200-2). In addition, the processing unit 23 of the information processing apparatus 20 may be the processing unit 210 of the communication device 200-2. Alternatively, the information processing apparatus 20 may be implemented by distributed processing of the association processing unit of the communication device 200-1 and the falling down determination processing unit of the communication device 200-2. For example, the multiple communication devices 200 illustrated in FIGS. 2 and 5 may be devices each serving as the information processing apparatus 20. For example, a program for executing the falling down determination processing and the like may be provided to each communication device 200 as application software of a smartphone.

In addition, the information processing apparatus 20 is not limited to any one of the server system 300 and the communication device 200, and may be implemented by distributed processing of the server system 300 and the communication device 200. The configurations having been described above are an example of the information processing system 10 and the information processing apparatus 20, and their specific configurations can be modified in various ways.

Further, the method of this embodiment is applicable to an information processing method that executes the following steps. The information processing method includes the steps of: acquiring information that associates sensor information, output from the wearable module 100, with location information identifying a location where the communication device 200 having received the sensor information is disposed; performing, based on the location information and the sensor information, the falling down determination processing for making a determination on the risk of falling down of a care recipient who wears the wearable module 100; and performing, based on the falling down determination processing, at least one of notification to the caregiver terminal 400 of a caregiver who provides assistance to the care recipient and control over the peripheral device 700 located around the care recipient. Further, in the information processing method, in the step of performing the falling down determination processing, the falling down determination processing according to the location where the communication device 200 is disposed is performed based on the location information.

In addition, a part of or all of the processing performed by the information processing apparatus 20 of this embodiment may be implemented by a program. The processing performed by the information processing apparatus 20 is the processing performed by the processing unit 210 and the processing unit 310, for example.

The program according to this embodiment can be stored, for example, in a non-transient information memory device (information storage medium) which is a computer-readable medium. The information memory device can be implemented by an optical disc, a memory card, an HDD, or a semiconductor memory, for example. The semiconductor memory is a ROM, for example. The processing unit 210 and the like perform various kinds of processing of this embodiment based on the program stored in the information memory device. In other words, the information memory device stores the program for causing a computer to function as the processing unit 210 and the like. The computer is a device including an input device, a processing unit, a storage unit, and an output unit. Specifically, the program according to this embodiment is a program for causing the computer to execute steps which will be described later using figures such as FIG. 9.

### 2. Falling Down Determination Processing

Next, the falling down determination processing will be described in detail as an example of the intervention determination processing. Note that, hereinbelow, a description will be given mainly of the falling down determination processing based on sensor information output from the acceleration sensor 120 of the wearable module 100. However, as will be described later using FIG. 18A, the falling down determination processing may be performed based on sensor information output from other devices such as the pressure sensors Se1 to Se4.

### 2.1 Processing Flow

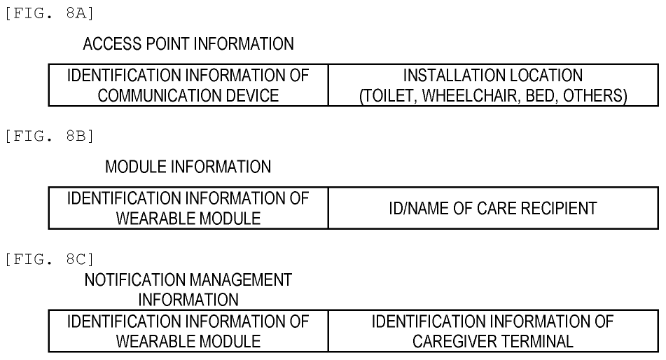

Hereinbelow, the processing of this embodiment will be described. In this embodiment, the processing may be executed by firstly executing a registration phase of registering information necessary for the processing and then executing a use phase corresponding to an actual assistance scene. Hereinbelow, processing in the registration phase will be described using FIGS. 7A to 8C, and processing in the use phase will be described using FIG. 9. Note that, although a description will be given of an example where the information processing apparatus 20 is the server system 300, a part of or all of the processing may be executed by the communication device 200 as described previously.

In the method of this embodiment, first registration processing of associating the communication device 200 and the arrangement location with each other and second registration processing of connecting the communication device 200 and the wearable module 100 to each other are performed. The second registration processing may be pairing in Bluetooth or may be processing of causing the wearable module 100 to store the SSID and password of wireless LAN. In addition, the second registration processing may include processing of registering the wearable module 100, paired with the communication device 200, in the system.

FIGS. 7A and 7B illustrate an example of a User Interface (UI) used for registration, and illustrate an example of a registration screen that is displayed on the display 240 by the operation of the processing unit 210 of the communication device 200 according to application software. FIG. 7A illustrates a screen used for the first registration processing and FIG. 7B illustrates a screen used for the second registration processing.

For example, the screens in FIGS. 7A and 7B may each include, in a lower part of the screen, an object OB1 that is an access point registration button and an object OB2 that is a sensor pairing setting button. The screen in FIG. 7A is displayed when the user performs an operation of selecting the object OB1, and the screen in FIG. 7B is displayed when the user performs an operation of selecting the object OB2. However, the configuration of the screen used in the registration phase is not limited to those of FIGS. 7A and 7B, and can be modified in various ways.

In the case of performing the first registration processing, for example, the user installs the above application software in the device used as the communication device 200 according to this embodiment, and then boots the application software to display the screen illustrated in FIG. 7A on the display 240. Then, in a state where the object OB1 is selected, the user selects the location where the communication device 200 is to be used. For example, the screen illustrated in FIG. 7A may include a text “select location to install this terminal” for prompting the user to select the location, and four radio buttons corresponding respectively to the toilet, the wheelchair, the bed, and others. The user can select any one of the four radio buttons.

For example, once the user selects the radio button corresponding to the toilet, the processing of registering the communication device 200, which the user is operating, as the communication device 200 disposed in the toilet. The same goes for the case of selecting the radio buttons other than that for the toilet, and the processing of registering the communication device 200, which the user is operating, as the communication device 200 disposed in the selected location.

Note that, as illustrated in FIG. 7A, in the case of selecting others, the user may input additional information on that location using a text box. Specifically, others indicate locations where the user walks. For example, the user may input a text indicating a specific location where the target communication device 200 is disposed, such as a corridor, stairs, and a dining room.

For example, in a case where an operation of selecting any of the locations is performed, the communication device 200 sends, to the server system 300, the identification information of the communication device 200 and information identifying the location selected by the user while associating them with each other. The server system 300 stores the received information in the storage unit 320 as access point information. FIG. 8A is a diagram illustrating access point information managed by the server system 300. As illustrated in FIG. 8A, the access point information is information associating identification information, identifying the communication device 200 according to this embodiment, with a location where this communication device 200 is disposed. More specifically, the access point information may be information associating the identification information of the communication device 200 with flag information. Note that, the access point information may include other information such as information identifying a facility where the communication device 200 is disposed, information identifying a user who made registration, and the registration date. In addition, the access point information may include additional information that is input using the above text box. This makes it possible to manage the device used as the communication device 200 according to this embodiment and the location where this device is disposed while associating them with each other. Note that, flag information indicating the location input using FIG. 7A may be stored in the storage unit 220 of the communication device 200.

Meanwhile, in the case of performing the second registration processing, for example, the user boots the above application software in the device used as the communication device 200 according to this embodiment to display the screen illustrated in FIG. 7B on the display 240. Then, in a state where the object OB2 is selected, the user selects the wearable module 100 which is a pairing target.

For example, the second registration processing may be executed when the new wearable module 100 is installed in a location such as a nursing care facility. The user turns on the power of the wearable module 100 and sets it to a state where pairing with the communication device 200 is possible. For example, in the case of using Bluetooth, the user sets the wearable module 100 to a pairing standby state.

The screen illustrated in FIG. 7B includes an object OB3 indicating a switch for controlling on/off of Bluetooth of the communication device 200, and a region RE1 for displaying sensors as pairing target candidates in a list form. For example, the user uses the object OB3 to turn on Bluetooth of the communication device 200. As a result, the communicator 230 of the communication device 200 searches for a device which exists around it and with which pairing is possible, and displays the search result in the region RE1.

As illustrated in FIG. 7B, the region RE1 may include a name (sensor name) of the connectable wearable module 100 and information indicating the connection state between the wearable module 100 and the communication device 200. In the example of FIG. 7B, through the search, the wearable modules 100 including at least two sensors of a sensor XXX and a sensor YYY have been searched out. The communication device 200 has been connected to the sensor XXX and is not connected to the sensor YYY.

In this case, the communication device 200 displays a text “connected” in the sensor XXX's state field. Meanwhile, the communication device 200 displays a text “not connected” in the sensor YYY's state field. In addition, as illustrated in FIG. 7B, the region RE1 may include an object for changing the connection state of each wearable module 100. For example, the display 240 of the communication device 200 displays an object OB4, indicating a disconnection button for disconnecting the connection, for the sensor XXX in the “connected” state. Meanwhile, the display 240 displays an object OB5, indicating a connection button for establishing a connection, for the sensor YYY in the “not connected” state. Based on the result of operation on these objects, the communicator 230 of the communication device 200 performs control to communicate with each wearable module 100. For example, when the object OB4 is selected, the communicator 230 disconnects the connection with the sensor XXX. When the object OB5 is selected, the communicator 230 executes a pairing sequence with the sensor YYY.

This makes it possible to control the communication state between the communication device 200 and the wearable module 100. For example, when the new wearable module 100 (such as the sensor YYY) is added, the communication device 200 and the wearable module 100 become able to communicate with each other, so that the communication device 200 becomes able to receive, as an access point, sensor information of the target wearable module 100. However, the timing of performing the second registration processing is not limited to the timing when the wearable module 100 is installed, and the second registration processing may be executed at any timing.

In addition, the processing unit 310 of the server system 300 may perform processing of storing the wearable module 100, paired with the communication device 200, in relation to the second registration processing. For example, in a case where the connection/disconnection state changes by selection of the object OB4 or the object OB5, the communication device 200 may send, to the server system 300, the identification information of the communication device 200 and identification information identifying the wearable module 100 paired with this communication device 200.

The server system 300 stores the identification information of the communication device 200 and the identification information of the wearable module 100 while associating them with each other. This makes it possible to manage the wearable module 100 newly added to the information processing system 10 and manage the communication device 200 accessible by the wearable module 100.

Note that, in a case where it is determined which of the bed 510, the wheelchair 520, the wheeled walker 540, the toilet 600, the dining room, the living room, and others (such as walking) corresponds to a care recipient in the example of FIG. 2, the wearable module 100 is preferably able to communicate with the communication devices 200-1 to 200-6. For example, when the new wearable module 100 is installed in a nursing care facility, the above second registration processing of performing pairing between the wearable module 100 and each of the communication devices 200-1 to 200-6 may be executed. However, the second registration processing for all the communication devices 200 is not necessary, and the second registration processing for a part of the communication devices 200 may be omitted. For example, in the case of a care recipient who does not need so much assistance in the toilet 600, the second registration processing for the communication device 200-4 may be omitted.

Alternatively, the second registration processing may be executed upon transmission of connection information, used for connection with the communication device 200, to the wearable module 100. For example, the device such as the server system 300 may collectively manage the SSIDs and passwords of the communication devices 200 and transmit the SSIDs and passwords to the wearable module 100 newly registered. For example, when the wearable module 100 is registered in the system through pairing between this wearable module 100 and the communication device 200-1, the server system 300 may transmit the SSIDs and passwords of the communication devices 200-2 to 200-6 to this wearable module 100. This can reduce the burden on the user at the time of registration.

In addition, in this embodiment, third registration processing of registering the identification information of the wearable module 100 and a care recipient who wears this wearable module 100 while associating them with each other may be executed. The third registration processing may be executed by a caregiver using the caregiver terminal 400 (mobile terminal device 410), for example. Here, application software to be installed in the communication device 200 and application software to be installed in the mobile terminal device 410 may be the same or different from each other.

FIG. 7C illustrates an example of a screen used for the third registration processing, and this screen is displayed, for example, on the display of the mobile terminal device 410 as described previously. The screen illustrated in FIG. 7C may include regions RE2 to RE4. In the region RE2, buttons for selecting the location where the communication device 200 is disposed are arranged. In the example of FIG. 7C, the buttons corresponding to the four locations, i.e., the toilet 600, the wheelchair 520, the bed 510, and others are displayed.

In the region RE3, when any of the locations is selected using the region RE2, the wearable modules 100 paired with the communication device 200 disposed in the selected location are displayed in a list form. For example, in a case where the information on the paired communication device 200 and wearable module 100 is stored in the server system 300 in relation to the second registration processing, a list of the wearable modules 100 to be displayed in the region RE3 is determined based on this information. In addition, in the region RE3, information on care recipients associated with the wearable modules 100 is displayed based on module information.

FIG. 8B illustrates an example of module information. The module information includes identification information identifying the wearable module 100 and information identifying a care recipient who uses this wearable module 100. The identification information of the wearable module 100 is the MAC address of the communication module 130, for example, but other information may be used instead. In addition, the information identifying a care recipient may be the name of the care recipient or may be other information such as the ID. Note that, the module information may include other information such as the identification information of the paired communication device 200, information identifying a facility where the wearable module 100 is installed, information identifying a user who made registration, and the registration date.

As illustrated in FIG. 7C, for the wearable modules 100 having been associated with care recipients (the sensor XXX and the sensor YYY in the example of FIG. 7), information on the care recipients associated with these wearable modules (e.g. Mr./Ms. AAA and Mr./Ms. BBB which are user names) is displayed based on the module information. On the other hand, for the wearable module 100 not having been associated with a care recipient yet, the term “not registered” is displayed.

The user selects any of the wearable modules 100 in the region RE3, and then performs, using the region RE4, an operation of changing or newly registering a care recipient with whom this wearable module 100 is associated. For example, in the region RE4, information on a care recipient who is a user of a facility is displayed. As an example, a list of care recipients is displayed in the region RE4, and when any of them is selected, detailed information on the care recipient illustrated in FIG. 7C is displayed. For example, the region RE4 includes a registration button, and when the user selects the registration button, processing of associating the wearable module 100 selected in the region RE3 and the care recipient displayed in the region RE4 is performed. Specifically, the mobile terminal device 410 sends, to the server system 300, the identification information of the wearable module 100 and the information of the care recipient while associating them with each other. The processing unit 310 of the server system 300 performs processing of updating the module information of FIG. 8B based on the information transmitted from the mobile terminal device 410.

Note that, in this embodiment, information on one wearable module 100 may be managed as data that varies from one location to another. For example, data on the sensor zzz registered in association with the toilet and data on the sensor ZZZ registered in association with the wheelchair may exist. However, since these sensors ZZZ indicate the same wearable module 100, the care recipient who is associated with them is possibly the same. Accordingly, even if the communication devices 200 as pairing targets are different, the third registration processing may be executed in a batch if the wearable module 100 is the same. For example, FIG. 7C illustrates the example where the sensor ZZZ is paired with the communication device 200 disposed in the toilet and the sensor ZZZ is associated with Mr./Ms. CCC. In this case, even for a communication device 200 which is a pairing target and is disposed in a location other than the toilet, the processing of associating the data with Mr./Ms. CCC is executed in a batch the sensor ZZZ is the same.

In addition, in consideration of notification to the caregiver terminal 400 described above using FIG. 5, the method of this embodiment may include fourth registration processing of registering the wearable module 100 and the caregiver terminal 400, to which notification of information based on this wearable module 100 is given, in association with each other.

For example, the user transmits, using the mobile terminal device 410, information associating a care recipient with the caregiver terminal 400 to which notification of information on this care recipient is given. For example, the application software may communicate with the server system 300 to display, in a list form, the wearable modules 100 registered in the second registration processing or care recipients associated with these wearable modules 100. For example, in a state where a given caregiver logins to the system using his/her ID and password, the caregiver may select one or more wearable modules 100 in the list. The mobile terminal device 410 sends, to the server system 300, information identifying the caregiver during login and information identifying the selected wearable modules 100. In addition, in a case where the caregiver uses the multiple caregiver terminals 400, the caregiver may make an input for specifying the caregiver terminals 400 which serve as notification targets.

The processing unit 310 of the server system 300 performs processing of updating notification management information illustrated in FIG. 8C based on the received information. As illustrated in FIG. 8C, the notification management information includes the identification information of wearable the module 100 and the identification information of the caregiver terminal 400 to which notification of information based on this wearable module 100 is given. The identification information of the caregiver terminal 400 may be SIM information, may be a MAC address, or may be other information. Note that, the identification information of the wearable module 100 may be replaced by information on the corresponding care recipient. In addition, the identification information of the caregiver terminal 400 may be replaced by information on the corresponding caregiver. Further, the notification management information may include information such as additional information representing more detailed notification condition.

As described above, the storage unit 320 of the server system 300 may store information such as the access point information, the module information, and the notification management information based on the first registration processing to the fourth registration processing. By using these pieces of information, the processing unit 310 can appropriately manage the devices in the information processing system 10 illustrated in FIGS. 2 and 5.

FIG. 9 is a sequence diagram illustrating processing in the information processing system 10 in the use phase that comes after the end of the registration phase described above. First, at Step S101, the wearable module 100 determines whether the connectable communication device 200 exists nearby. For example, the processing of Step S101 may be processing of executing a sequence including transmission and reception of Bluetooth advertising packets or may be processing of executing a sequence including SSID scan in wireless LAN.

If the connectable communication device 200 exists, at Step S102, connection between the wearable module 100 and the communication device 200 is established.

Information necessary for establishing connection has been acquired already in the second registration processing described above, for example. Accordingly, when the registered communication device 200 exists within a predetermined distance, the wearable module 100 establishes connection with this communication device 200.

At Step S103, the wearable module 100 transmits sensor information, detected by the acceleration sensor 120, to the communication device 200 using the communication module 130. The processing of Step S103 is executed regularly at predetermined intervals, for example. Note that, the sensor information may include identification information identifying the wearable module 100 from which this sensor information is transmitted.

At Step S104, the communication device 200 performs processing of associating the sensor information received at Step S103 with the identification information of the communication device 200. Then, at Step S105, the communication device transmits the associated information to the server system 300. For example, the communication device 200 transmits the identification information of the wearable module 100, the sensor information, and the identification information of the communication device 200 while associating them with each other.

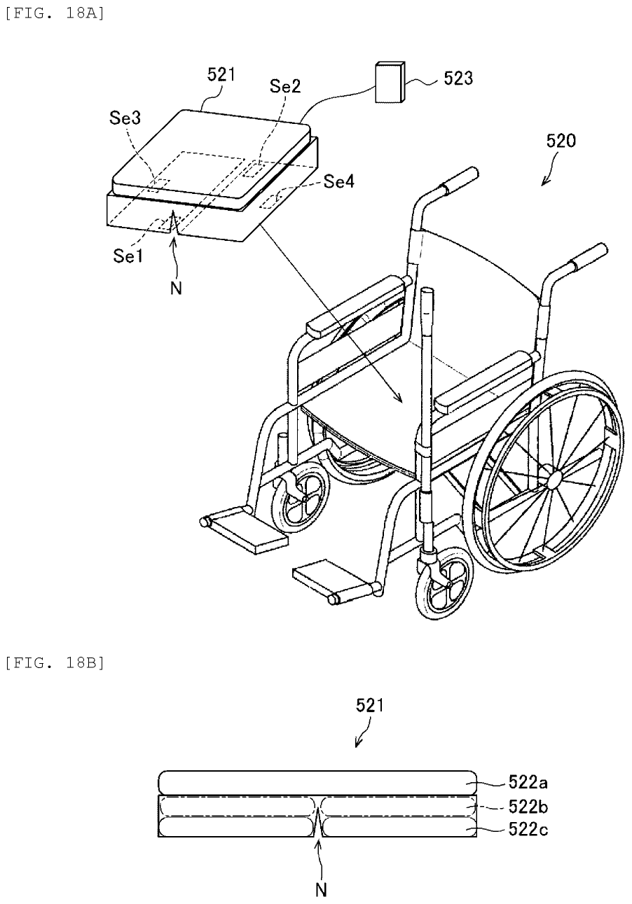

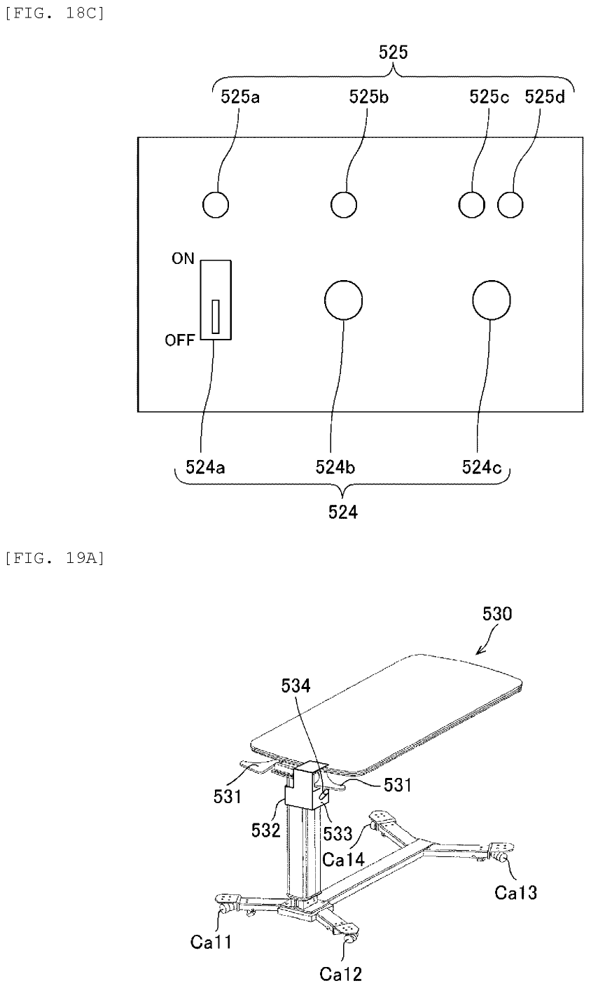

At Step S106, based on the received information, the server system 300 executes determination processing according to the location of a care recipient. For example, the processing unit 310 identifies flag information based on the acquired identification information of the communication device 200 and the access point information illustrated in FIG. 8A. Then, the processing unit 310 executes the determination processing according to the location of a care recipient based on the flag information and the sensor information. Note that, the determination processing may be the falling down determination processing. The falling down determination processing will be described later in detail using FIGS. 16 and 17. For example, as will be described later, the output of the falling down determination processing is information indicating accuracy on whether the risk of falling down exists.

If determining that the risk of falling down exists, at Step S107, the processing unit 310 identifies the caregiver terminal 400 to which notification of the risk of falling down is given. Specifically, the processing unit 310 identifies the caregiver terminal 400 as a notification target based on the identification information of the wearable module 100 acquired at Step S105 and the notification management information of FIG. 8C. For example, the processing unit 310 may acquire the Internet Protocol (IP) address of the caregiver terminal 400 as a notification target.

At Step S108, the server system 300 notifies the identified caregiver terminal 400 of information on the risk of falling down. The information notified here may include information indicating that the risk of falling down exists, such as a text that “Mr./Ms. AAA is likely to fall down in the toilet”, the name of a care recipient who has the risk of falling down, and the location of this care recipient. In addition, notification aspects can be modified in various ways, and notification may be given by displaying a text on the display of the mobile terminal device 410 or outputting voice to the headset 420. Alternatively, notification may be given through emission of LED light or vibrations and the like using a motor.

In addition, FIG. 9 illustrates the example where the server system 300 gives push notification to the caregiver terminal 400 if detecting the risk of falling down. However, the method for a caregiver to acquire the determination result based on information such as the sensor information is not limited to this, and the caregiver may actively acquire the determination result by operating the caregiver terminal 400.

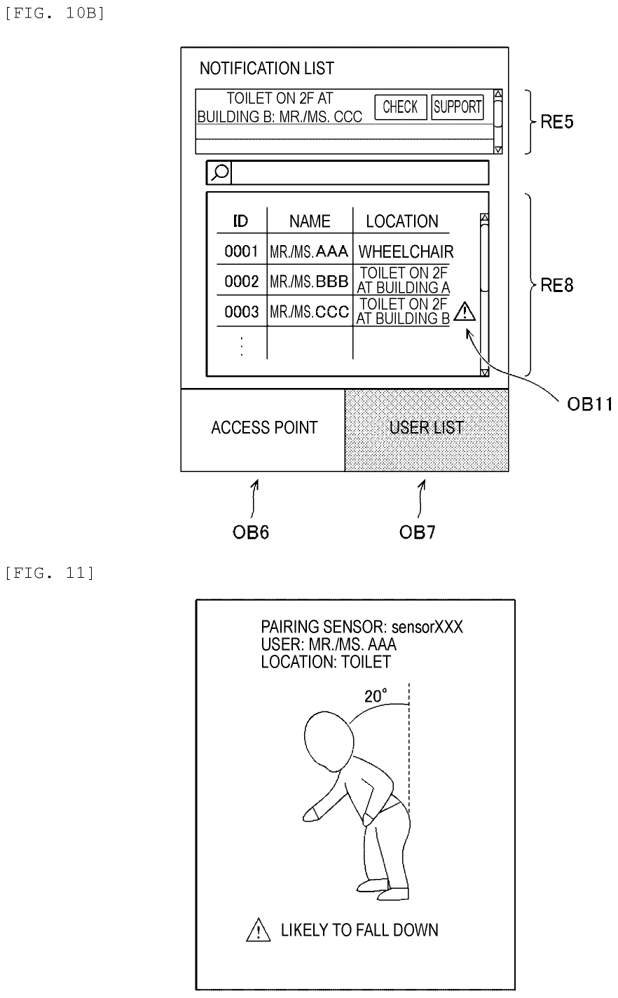

FIGS. 10A and 10B illustrate an example of screens displayed on the display of the mobile terminal device 410, for example. These screens may be displayed by the application software that performs the first registration processing to the fourth registration processing or may be displayed by other application software.

The mobile terminal device 410 may display information on a per-communication device 200 basis and display information on a per-care recipient basis. For example, the screens of FIGS. 10A and 10B may include, in a lower part of the screen, an object OB6 that is a button for displaying information on a per-access point basis and an object OB7 that is a button for displaying information on a per-care recipient basis. The screen of FIG. 10A is displayed when the user selects the object OB6, and the screen of FIG. 10B is displayed when the user selects the object OB7. However, the screen configuration used for viewing the result of the falling down determination processing is not limited to that of FIGS. 10A and 10B, and can be modified in various ways.

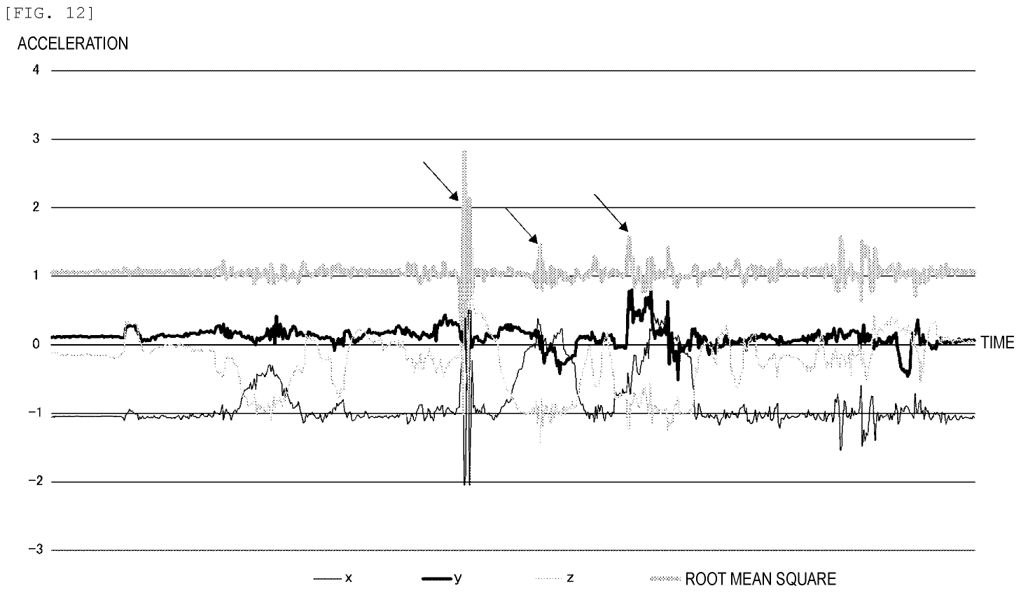

As illustrated in FIG. 10A, the screen for displaying information on a per-communication device 200 basis may include regions RE5 to RE7. The region RE5 is a region for displaying notification. For example, if there is a care recipient determined as having a high risk of falling down, the location where the communication device 200 is disposed and the name of the care recipient are displayed in the region RE5. In the region RE5, an object OB8 that indicates a check button and an object OB9 that indicates a support button may be displayed. When the object OB8 is selected, the display of the mobile terminal device 410 transitions to a screen for displaying detailed information on the risk of falling down. The screen for displaying the detailed information is a screen which will be described later using FIG. 11, for example. On the other hand, when the object OB9 is selected, the mobile terminal device 410 sends, to the processing unit 310, information indicating that a caregiver associated with this mobile terminal device 410 is in charge of support on the risk of falling down. Once the caregiver to provide support is determined, the processing unit 310 may exclude the information on the risk of falling down from the display. To put it differently, the information displayed in the region RE5 may be information on the risk of falling down for which the caregiver to provide support has not been determined yet. However, information on the risk of falling down for which the caregiver to provide support has been determined already may be displayed together with the fact that the caregiver has been determined and the name of the caregiver in charge, and specific display aspects can be modified in various ways.

Meanwhile, in the region RE6, buttons for selecting the location where the communication device 200 is disposed are arranged. In the example of FIG. 7C, the buttons corresponding to the four locations, i.e., the toilet 600, the wheelchair 520, the bed 510, and others are displayed.

In the region RE7, when any of the locations is selected using the region RE6, information on the communication device 200 disposed in the selected location is displayed in a list form. In the example of FIG. 10A, the toilets 600 are located at four locations, i.e., on the first floor of Building A, on the second floor of Building A, on the first floor of Building B, and on the second floor of Building B in a) care facility, and the communication devices 200 different from each other are arranged at these locations, respectively. Note that, in the falling down determination processing, these toilets 600 do not need to be distinguished from each other; however, from the perspective of more smooth check and intervention by a caregiver, information including the location of each toilet 600 and the like may be stored in the storage unit 320 and the like.

In this case, in the region RE7, information on the four communication devices 200 is displayed. For example, in the region RE7, the names of care recipients located near the communication device 200 are displayed together with the information identifying the buildings and floors where the communication device 200 is disposed. The processing unit 310 performs control to display the screen illustrated in FIG. 10A by referring to whether the wearable module 100 transmitting sensor information to the communication device 200 exists and, if the wearable module 100 exists, information on a care recipient who is associated with this wearable module (module information of FIG. 8B). In the example of FIG. 10A, a state where Mr./Ms. BBB is located in the toilet 600 on the second floor of Building A, Mr./Ms. CCC is located in the toilet 600 on the second floor of Building B, and nobody is located in the remaining two toilets 600 is presented to a caregiver.

Note that, in a case where there is a care recipient determined as having the risk of falling down, the fact that such a care recipient exists may be displayed in the region RE7. For example, in the example of FIG. 10A, as illustrated in the region RE5, the risk of falling down of Mr./Ms. CCC is detected in the toilet on the second floor of Building B. Accordingly, in the region RE7, an object OB10 indicating an alarm may be displayed in association with data corresponding to Mr./Ms. CCC.

As illustrated in FIG. 10B, the screen for displaying information on a per-care recipient basis may include the region RE5 and a region RE8. The region RE5 is a region indicating notification as in FIG. 10A.

In the example of FIG. 10B, in the region RE8, information on at least three or more care recipients including Mr./Ms. AAA, BBB, and CCC is displayed. Here, the care recipients to be displayed may be all care recipients using a nursing care facility or may be care recipients to whom a caregiver using the mobile terminal device 410 is in charge of assistance. In the example of FIG. 10B, information included in the region RE8 is information on the ID uniquely identifying a care recipient, the name of the care recipient, and the location of the care recipient. For example, based on information from the communication device 200, the processing unit 310 can identify the communication device 200 to which each wearable module 100 is connected. Accordingly, the processing unit 310 can identify the location of a care recipient based on the information identifying the communication device 200 to which the wearable module 100 is connected, the access point information of FIG. 8A, and the module information of FIG. 8B. In the example of FIG. 10B, information indicating that Mr./Ms. AAA is located in the wheelchair 520, Mr./Ms. BBB is located in the toilet on the second floor of Building A, and Mr./MS. CCC is located in the toilet on the second floor of Building B is displayed in the region RE8. Note that, in the region RE8, as in the region RE7 of FIG. 10A, an object OB11 indicating an alarm may be displayed in association with data corresponding to Mr./Ms. CCC.

FIG. 11 illustrates an example of a presentation screen displayed when the object OB8 is selected in FIGS. 10A and 10B. For example, the presentation screen may include information identifying the wearable module 100, the name of a care recipient, information identifying the location, and information on the risk of falling down. In the example of FIG. 11, the sensor XXX is the identification information of the wearable module 100 and Mr./Ms. AAA is the name of a care recipient who wears the wearable module. In other words, FIG. 11 illustrates the example of the presentation screen showing, to a caregiver, a situation of a care recipient who wears the sensor XXX in the toilet.

For example, in a case where the falling down determination processing is processing of obtaining a deviation from a reference posture by an angle, as illustrated in FIG. 11, the presentation screen may be a screen for displaying the angle using a pictogram. By using the screen illustrated in FIG. 11, for example, a situation where the care recipient is leaning forward by approximately 20 degrees relative to an upright posture can be presented to the caregiver in a way easy to understand. In addition, the presentation screen may include information such as a text indicating whether the risk of falling down exists, e.g., a text “likely to fall down” and a text “not likely to fall down”. Note that, the text to be displayed is not limited to this. For example, in a case where his/her motion characteristics in the toilet change, a text indicating information such as a prediction of falling down in the future, the specific location of falling down, and the situation of falling down, e.g. a text “the risk of falling down is increasing, the user is highly likely to fall down in the toilet” may be displayed. Likewise, during walking, based on a change in his/her walking motion characteristics, information such as a text “the risk of falling down is increasing, the user is highly likely to fall down at an uneven location” may be displayed. In addition, in a case where a camera is disposed at a target location, although privacy needs to be taken into consideration, display of an image taken by the camera is not precluded.

Note that, the information displayed using FIG. 11 may be real-time information or may be information indicating past history of the target location and care recipient. By presenting the information illustrated in FIGS. 10A to 11, it is possible to present to a caregiver where a care recipient is located and what situation the care recipient is in, in a way easy to understand. In addition, in the screen of FIG. 11, simulation information predicting a future falling down situation may be displayed. For example, the processing unit 310 may perform processing of predicting a change in the posture of a care recipient based on sensor information acquired when determining that the risk of falling down exists, and display a prediction result on the display of the mobile terminal device 410. For example, based on the sensor information corresponding to a predetermined period of time (e.g. three seconds), the processing unit 310 presumes a posture that a care recipient takes at a later timing. This makes it possible to present how the care recipient will fall down in a falling down accident that is likely to occur in the future. For example, if the care recipient is predicted to fall down in such a way that he/she hits his/her head hard, it is possible to prompt a caregiver to deal with this beforehand. Meanwhile, if the care recipient is predicted to fall down in such a way that he/she slowly falls on his/her bottom, since such a fall involves a relatively low risk of getting hurt, the caregiver can determine that other more urgent assistance should be prioritized, for example.

In addition, in the method of this embodiment, information on whether a care recipient gets hurt seriously by falling down, such as a text “possibility of hitting head” and a text “possibility of femur fracture” may be output. For example, as described previously, in this embodiment, it is possible to presume a location where falling down has occurred/is likely to occur, and presume the posture and direction of falling down of the care recipient at the time of falling down based on an output from the acceleration sensor 120 and the like. Accordingly, the processing unit 310 may perform processing of presuming, as information on the risk of falling down, whether a severe injury occurs at a specific portion or presuming a probability value indicating accuracy on whether this injury occurs, for example. In addition, the information thus obtained may be displayed using the screen of FIG. 11, for example. For example, in a case where a staff such as a caregiver is not present at the site where falling down has occurred, the caregiver has heretofore determined how to deal with this, including whether thorough checkup is needed, by hearing a hit portion and the like from the care recipient, checking whether there is an injury and the position of the injury, and observing the behavior of the care recipient, for example. Since the degree of accuracy of this determination depends on the degree of proficiency of the caregiver, the caregiver with a low degree of proficiency may make erroneous determination, so that thorough checkup may not be able to be performed appropriately, for example. In that respect, according to this embodiment, it is possible to present the possibility of an occurrence of a serious injury to the caregiver who is not on site, and thus possible to help the caregiver provide appropriate support irrespective of his/her degree of proficiency. In addition, in this embodiment, if it is determined that there is a “possibility of hitting head” or “possibility of femur fracture”, thorough checkup may be prompted to the caregiver and the like, or processing of automatically arranging thorough checkup and the like may be performed.

Note that, at least one of algorithms and parameters used for processing of obtaining the “possibility of hitting head”, the “possibility of femur fracture”, and the like may be changed depending on the location. For example, depending on whether a care recipient is in the toilet 600 or walking, the “possibility of hitting head” or “possibility of femur fracture” may be obtained using different determination methods. In addition, machine learning such as neural network (NN) may be employed for the processing of obtaining them. For example, training data may be generated by assigning ground truth data, with a probability value indicating the “possibility of hitting head” set to 1, to sensor information and location information acquired when the care recipient actually hits his/her head by falling down. Through the machine learning based on such training data, it is possible to generate learned model that outputs a probability value indicating the “possibility of hitting head”. Further, as to display of the possibility of an occurrence of a serious injury described above, to show/hide the data may be determined on a per-caregiver basis or on a per-nursing care facility basis. For example, the display items in FIG. 11 and the like may be settable, and a person in charge of an assistance facility may change the status of showing/hiding the possibility of an occurrence of a serious injury.

### 2.2 Falling Down Determination According to Location

Next, a specific example of the falling down determination processing will be described.

### 2.2.1 Example of Specific Situation of Falling Down

Falling down in a nursing care facility can occur at various locations, and a situation under which falling down occurs, the cause of falling down, and how falling down occurs are different from one location to another. Hereinbelow, a description will be given of falling down from the bed 510, falling down from the wheelchair 520, falling down in the toilet 600, and falling down during walking. Note that, falling down following description is not limited to falling down in a narrow sense, e.g. a body falls on a floor surface or the like, and may include a state of losing a balance compared to a normal state.

First, a description will be given of falling down from the bed 510. At the bed 510, falling down may occur when a care recipient tries to stand up while sitting on the edge of the bed. In a normal condition, the care recipient first lowers his/her head and shifts to a state of bearing weight on his/her feet by exerting his/her strength on his/her knees, a bed surface, a handrail, or the like with his/her hands thereon, and then stands up while raising his/her head. However, if the care recipient tries to stand up without sufficient strength on his/her feet and without his/her center of gravity moving forward, the care recipient will lose a balance backward, resulting in sitting back with great force on the bed surface. Meanwhile, if his/her center of gravity moves forward excessively at the time of placing weight on his/her feet, the care recipient will fall forward (forward fall). Meanwhile, if his/her hand slips off at the time of placing his/her hands thereon, the care recipient may fall on the floor surface from the side of the hand having slipped off.

Also, falling down may occur also when a care recipient sits on a bed surface from a standing position. In a normal condition, the care recipient first places one hand on the bed surface while facing the bed 510, then turns his/her body halfway to face the opposite direction of the bed 510, and then places his/her buttocks on the bed surface. However, if the hand on the bed surface slips off, the care recipient cannot support his/her weight, thus resulting in falling down. Meanwhile, if the care recipient sits down without carefully checking the bed surface when he/she turns his/her body halfway with his/her hand on the bed surface, his/her buttocks may fail to ride on the bed surface in the first place, or even when his/her buttocks successfully ride on the bed surface once, they may slide off because he/she sits down too shallowly.

Meanwhile, falling down may occur when a care recipient moves from the bed 510 to the wheelchair 520 or the like. Note that, falling down at the time of movement between them mentioned here is deemed as falling down from the bed 510. For example, falling down occurs when the brake of the wheelchair 520 to which the care recipient is to move is not applied or when the care recipient accidentally releases the brake. For example, the wheelchair 520 may move at a phase where the care recipient places one hand on an arm support or the like of the wheelchair 520 and puts his/her weight on it, which may lead to falling down. Alternatively, the wheelchair 520 may move backward at a phase where the care recipient tries to sit on the seat of the wheelchair 520, causing the care recipient to fall on his/her buttocks.

Next, a description will be given of falling down from the wheelchair 520. While riding on the wheelchair 520, a care recipient may feel pain on his/her buttocks because keeping the same posture and shift his/her buttocks gradually forward on the seat. In this case, as the amount of shift increases, the care recipient becomes in a state of sitting shallowly, and finally his/her buttocks fall off a seat surface, resulting in falling down. Meanwhile, during moving with the wheelchair 520, the care recipient puts his/her feet on foot supports, and if the care recipient tries to stand without removing his/her feet from the foot supports, the wheelchair 520 itself may tilt forward, causing the care recipient to fall forward. Alternatively, if the care recipient accidentally tries to stand up with the brake of the wheelchair 520 released, the wheelchair 520 moves backward while the care recipient is standing up, causing the care recipient to fall backward. Meanwhile, although not direct falling down, the wheelchair 520 may crash into a wall or furniture due to an operational error or the like and impact may be applied on the care recipient.

Next, a description will be given of falling down in the toilet 600. In the toilet 600, a care recipient first lifts a lid while facing a toilet bowl, turns his/her body halfway around, and then lowers his/her pants slightly and sits on the toilet bowl. For example, during the half turn of the body, falling down can occur due to his/her feet not moving properly. In addition, when sitting on the toilet bowl, the care recipient may fail to sit down and fall due to a narrow seat surface.

After sitting on the toilet bowl, the care recipient lowers his/her pants further, defecates, takes toilet paper and wipes himself/herself. Since the care recipient needs to tilt his/her body when lowering the pants further and when wiping with toilet paper, falling down occurs due to a loss of balance. In addition, since exerting abdominal pressure when defecating, the care recipient may faint due to rise of blood pressure. In this event, the care recipient may fall forward, or may fall backward in the absence of a backrest. Further, the care recipient may fall sideways.

Thereafter, the care recipient leaves the toilet 600 according to the opposite procedure from the above described procedure. Specifically, the care recipient pulls up his/her pants slightly, stands up while holding onto a handrail, and fully pulls up the pants. Then, the care recipient turns his/her body halfway to face the toilet bowl, flushes the stool away, closes the lid of the toilet bowl, and turns his/her body halfway again to leave the toilet 600. For example, during the turn of the body, falling down can occur due to his/her feet not moving properly.

Next, a description will be given of falling down during walking. During walking, a care recipient may fall forward when failing to move his/her feet forward or when stumbling over something. In addition, the center of gravity of the care recipient may accidentally shift backward for some reason, and in this case, the care recipient falls backward. Further, if his/her legs cannot support his/her weight enough, one of the legs bends for example, causing the care recipient to fall down from the side of this leg.

### 2.2.2 Example of Sensor Information According to Location

As described above, a situation under which falling down occurs, the cause of falling down, and how falling down occurs are different depending on whether the care recipient is on the bed 510, on the wheelchair 520, at the toilet 600, or during walking. As a result, even if an event to be detected is falling down which is a common event, the tendency of sensor information of the acceleration sensor 120 varies depending on the location where the event has occurred.

FIGS. 12 to 15 illustrate examples of time series sensor information including information observed when falling down occurs. The sensor information here is an output from the acceleration sensor 120 mounted on the chest or back of a care recipient as illustrated in FIG. 2, and indicates acceleration in the x, y, and z axes and the root mean square of the 3-axis acceleration. In FIGS. 12 to 15, the horizontal axis represents the time, and the vertical axis represents acceleration (G). In addition, in each of the graphs, points assigned with arrows correspond to falling down.

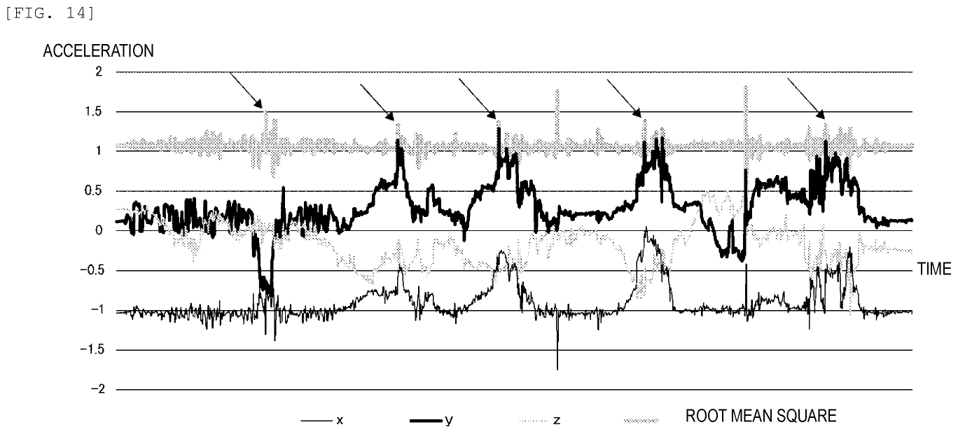

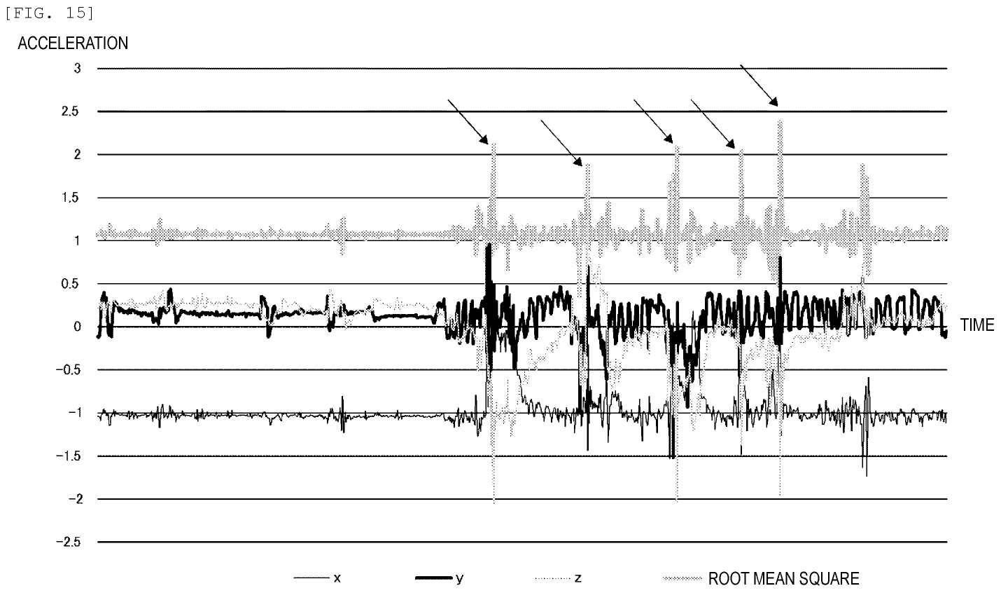

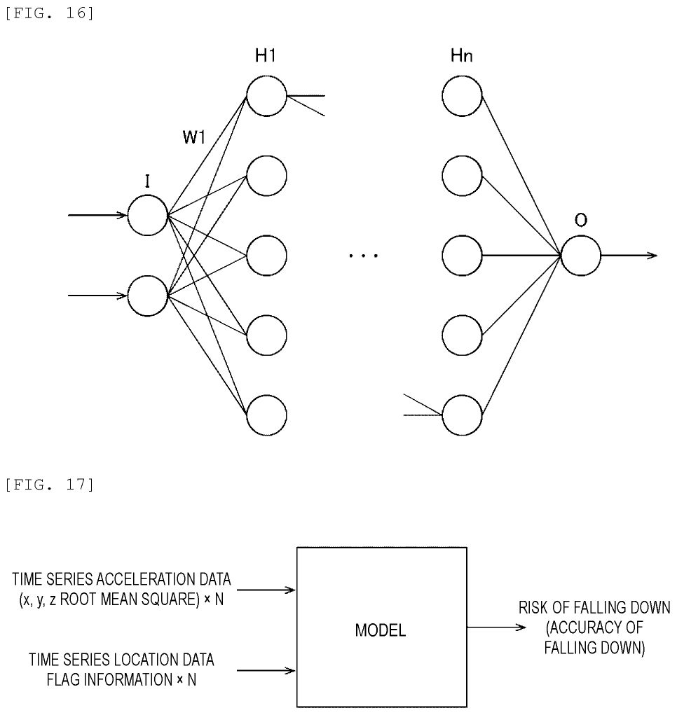

FIG. 12 illustrates the example of sensor information including falling down from the bed 510, FIG. 13 illustrates the example of sensor information including falling down from the wheelchair 520, FIG. 14 illustrates the example of sensor information including falling down in the toilet 600, and FIG. 15 illustrates the example of sensor information including falling down during walking.

For example, as can be understood from the root mean square in FIGS. 12 to 15, the magnitude of acceleration representing impact at the time of falling down varies depending on the location. For example, the example of the case of the bed 510 illustrated in FIG. 12 includes three times of falling down including one having a large root mean square value of around 2.8 G and one having a relatively small root mean square value of around 1.5 G. Accordingly, in a case where the processing of detecting an event of falling down from the bed is performed based on whether acceleration equal to or larger than a threshold is detected, for example, the values that vary from one to another need to be determined as falling down.

In the example of the case of the wheelchair 520 illustrated in FIG. 13, a root mean square value at the time of falling down is around 2.0 G. Accordingly, for determining whether falling down from the wheelchair 520 occurs based on threshold determination, a threshold that makes it possible to distinguish between the above value and a normal value can be set.

In the example of the case of the toilet 600 illustrated in FIG. 14, a root mean square value at the time of falling down is around 1.5 G or smaller. Accordingly, for determining whether falling down in the toilet 600 occurs based on threshold determination, a large threshold cannot be set unlike the case of other locations, and it is necessary to set a threshold that allows such relatively small impact to be detected as falling down.

In the example of the case of during walking illustrated in FIG. 15, a root mean square value at the time of falling down is around 2.0 G to 2.4 G. Accordingly, for determining whether falling down during walking occurs based on threshold determination, a threshold that makes it possible to distinguish between the above value and a normal value can be set. In addition, during walking, since a normal motion is large compared to those at the bed 510, the wheelchair 520, and the toilet 600, it is not preferable to set an excessively small threshold (such as a threshold smaller than 1.5 G) for preventing impact caused by normal walking from being falsely determined as falling down.

The foregoing description has been given only of the difference in terms of the magnitude of root mean square. However, as can be understood from the above description, posture conditions (such as the position of the center of gravity and the inclination of the body) observed before falling down occurs are also different from one location to another. Accordingly, it is conceivable that not only the root mean square value at one timing but also the tendency of time series change are different depending on the location. In addition, since the acceleration in the x, y, and z axes correspond respectively to the front-rear direction, the left-right direction, and the up-down direction of a care recipient, these are information representing the posture of the care recipient. Accordingly, the tendency of sensor information in terms of any of the x, y, and z axes and a combination of any two or more of them is also different depending on the location.

As described above, since the sensor information at the time of falling down is different from one location to another, processing precision can be improved by causing the falling down determination processing based on the sensor information to be performed according to the location.

### 2.2.3 Specific Example of Falling Down Determination Processing

As described previously, the falling down determination processing according to this embodiment may be processing of comparison between an acceleration value and a threshold. In this case, the storage unit 320 stores a threshold at each location, and at Step S107 of FIG. 9, the corresponding threshold is identified based on the location identification and result the falling down determination processing of comparing the threshold with sensor information is performed. However, the falling down determination processing is not limited to this.

For example, the processing unit 310 may perform the falling down determination processing using machine learning. Hereinbelow, a description will be given of an example of using a neural network as the machine learning. The neural network is hereinafter referred to as an NN. However, the machine learning is not limited to the NN, and other methods such as a support vector machine (SVM) method and a k-means method may be used, or a method obtained by developing these methods may be used. In addition, although the following example uses supervised learning, another machine learning such as unsupervised learning may be used.

FIG. 16 illustrates a basic configuration example of the NN. One circle in FIG. 16 is referred to as a node or neuron. In the example of FIG. 16, the NN includes an input layer, two or more intermediate layers, and an output layer. A reference sign I corresponds to the input layer, reference signs H1 and Hn correspond to the intermediate layers, and a reference sign O corresponds to the output layer. In addition, in the example of FIG. 16, the number of nodes of the input layer is two, the number of nodes of each intermediate layer is five, and the number of nodes of the output layer is one. However, various modifications are possible as the number of layers of the intermediate layer and the number of nodes in each layer. In addition, FIG. 16 illustrates an example where each node in a given layer is connected to all nodes in the next layer, but this configuration can also be modified in various ways.

The input layer receives an input value and outputs it to the intermediate layer H1. In the example of FIG. 16, the input layer I receives two kinds of input values. Note that, each node of the input layer may perform some sort of processing on the input value and output the value thus processed.

In the NN, a weight is assigned between two nodes connected to each other. A reference sign W1 of FIG. 16 indicates a weight assigned between the input layer I and a first intermediate layer H1. The reference sign W1 represents a set of weights assigned between a given node in the input layer and a given node in the first intermediate layer. For example, the reference sign W1 in FIG. 16 indicates information including ten weights.

In each node of the first intermediate layer H1, an output from the node of the input layer I connected to this node is weight added using the weight W1 and then added with bias. Further, at each node, an output from this node is obtained by applying an activating function, which is a nonlinear function, to the addition result. The activating function may be the ReLU function, may be the sigmoid function, or may be other functions.

Meanwhile, the same goes for the subsequent layers. Specifically, in a given layer, an output to the next layer is obtained in such a way that an output from the previous layer is weight added using the weight W, added with bias, and then applied with an activating function. The NN sets an output from the output layer as an output from the NN.

As can be understood from the above description, in order to obtain desired output data from input data using the NN, it is necessary to set an appropriate weight and bias. In the learning, training data obtained by associating given input data and ground truth data representing correct output data for this input data is prepared. Learning processing of the NN is processing of obtaining a weight which is most likely accurate based on training data. Note that, for the learning processing of the NN, various learning methods such as the Backpropagation method have been known. In this embodiment, since these learning methods can be widely employed, their detailed description will be omitted.

Further, the configuration of the NN is not limited to that illustrated in FIG. 16. For example, a network having other configurations such as a Recurrent neural network (RNN) may be used as the NN. The RNN may be a Long Short Term Memory (LSTM), for example. Alternatively, a Convolutional neural network (CNN) may be used as the NN.

FIG. 17 is a diagram illustrating the relationship between input data and output data in this embodiment. For example, the input data includes sensor information from the acceleration sensor 120 and flag information identifying the location. As described previously, the sensor information includes acceleration values in the x, y, and z axes and the root mean square value of the 3-axis acceleration values. However, all of these four values do not necessarily have to be used, and a part of these values may be omitted. In addition, the flag information may be 4-bit data each indicating any of the toilet 600, the bed 510, the wheelchair 520, and walking, for example. In the 4-bit data, “1000” represents the toilet 600, “0100” represents the bed 510, “0010” represents the wheelchair 520, and “0001” represents during walking, for example.

As illustrated in FIG. 17, in the method of this embodiment, the input data includes not only the sensor information from the acceleration sensor 120 but also the information identifying the location. This makes it possible to perform processing in consideration of influence on falling down that varies depending on the location. Specifically, the falling down determination processing according to the location can be implemented, and thus processing precision can be improved.

In addition, as illustrated in FIG. 17, the input data may be time series data. For example, in a case where the acceleration sensor 120 performs measurement once for every predetermined period of time and four acceleration values including acceleration values in the x, y, and z axes and the root mean square value of these values are obtained as one measurement result, the input data is a set of N×4 acceleration values acquired by the N-times measurement. N is an integer of two or more. Note that, the root mean square may be calculated by the acceleration sensor 120, or may be calculated by the processing unit 210 of the communication device 200 or the processing unit 310 of the server system 300.

In addition, as described previously, the flag information representing the location is information that is identified based on the identification information of the communication device 200, and the identification information of the communication device 200 may be assigned every time the communication device 200 receives the sensor information.

In this way, by setting time series data as the input data, it is possible to perform processing in consideration of time series change of the input data. For example, as described above, time series behaviors such as the background of falling down and how falling down occurs are different from one location to another. In that respect, by processing the time series input data using the LSTM or the like, it is possible to reflect time series difference depending on the location over the falling down determination processing.

In addition, the output data in the machine learning is information representing accuracy on whether the risk of falling down of a care recipient exists. For example, the output layer of the NN may output a probability value, equal to or larger than 0 and equal to or smaller than 1, as the output data. The larger this value is, it indicates that the risk of falling down has high probability, that is, the risk of falling down is high.

For example, the processing unit 310 of the server system 300 may acquire learned model that is generated by machine learning based on training data including sensor information for training output from the wearable module 100 and location information for training identifying a location where this sensor information for training has been acquired, and that is configured to output information indicating accuracy on the risk of falling down. For example, during a learning phase, the processing unit 310 acquires training data in which input data illustrated in FIG. 17 and ground truth data indicating whether the risk of falling down exists are associated with each other. The input data is data obtained by assigning flag information, indicating the location, to the sensor information of the acceleration sensor 120 acquired through the communication device 200 as described above. Note that, during the learning phase, the communication device 200 may be omitted, and the user may associate the flag information identifying the location with the sensor information. In addition, the ground truth data is information assigned by a skilled worker, for example. For example, the skilled worker may input information identifying the timing when the risk of falling down is so high that a caregiver should intervene. In this case, ground truth data indicating that the risk of falling down is high is associated with input data of a period of time corresponding to this timing (such as a predetermined period of time prior to this timing). Alternatively, ground truth data may be assigned based on history on whether a care recipient has actually fallen down. For example, in a case where a care recipient has actually fallen down, ground truth data indicating that the risk of falling down is high is associated with input data of a period of time corresponding to this timing.

Subsequently, the processing unit 310 performs processing of updating the weight of the NN. Specifically, the processing unit 310 inputs input data to the NN, and performs forward operation using the weight at this phase to obtain output data. The processing unit 310 obtains an objective function based on the output data and ground truth data. For example, the objective function mentioned here is an error function based on a difference between the output data and the ground truth data or an intersection entropy function based on the distribution of the output data and the distribution of the ground truth data. The processing unit 310 updates the weight so that the error function may be reduced, for example. For the method of updating the weight, methods such as the Backpropagation method described above have been known, and these methods can be widely employed in this embodiment.

The processing unit 310 terminates the learning processing if a given condition is satisfied. For example, the training data may be divided into learning data and validation data. The processing unit 310 may terminate the learning processing if the processing of updating the weight is performed using all pieces of the learning data, or may terminate the learning processing if the accuracy rate based on the validation data exceeds a given threshold. After the learning processing is terminated, the NN including the weight at this phase is stored in the storage unit 320 as the learned model. Note that, the learning processing is not limited to one executed by the server system 300, and may be executed by an external device. The server system 300 may acquire the learned model from the external device.

In addition, in a presumption phase, the processing unit 310 reads the learned model from the storage unit 320. Then, the processing unit 310 acquires input data obtained by assigning flag information to the sensor information of the acceleration sensor 120 acquired through the communication device 200, and inputs this input data to the learned model. The processing unit 310 performs forward operation based on the weight acquired by the learning processing to obtain output data. As described previously, the output data is numeric data indicating the level of the risk of falling down.

For example, in a case where a threshold Th in the range of 0<Th<1 is set in advance, the processing unit 310 may determine that the risk of falling down exists if a value of the output data is equal to or larger than Th. If it is determined that the risk of falling down exists, the processing at Steps 107 and 108 illustrated in FIG. 9 is executed.

### 2.2.4 Modified Example in Falling Down Determination Processing

### <Other Examples of Information Used for Falling Down Determination Processing (Assessment, Falling Down History)>

In FIG. 17, the description has been given of the example where the input data are the sensor information and the flag information identifying the location. However, the method of this embodiment is not limited to this, and the input data may include other information.

For example, the input data may include information indicating a classification result obtained by classifying users into several classes. For the classification, a device other than the wearable module 100 may be used.

For example, the following Uniform Resource Locator (URL) discloses Waltwin which is a device including an insole-type pressure sensor. The use of this device makes it possible to divide a sole into multiple portions and measure a sole pressure at each of these portions in real time, for example.
https://media.paramount.co.jp/service/rehabilitation/waltwin/

In addition, the following URL discloses SR AIR which is a device for measuring, in real time, a pressure at each of regions obtained by segmentalizing a target into 15×15 and the center of gravity of the target, for example. The use of this device makes it possible to make a detailed analysis of pressure change in each of situations such as a standing posture, a sitting posture, and walking of a care recipient.
http://www.fukoku-jp.net/srsoftvision/common/img/download/download_pdf_007.pdf

For example, the processing unit 310 classifies a care recipient into any of multiple classes based on factors such as the length of time during which the care recipient can keep the standing posture or the sitting posture, the direction in which the care recipient is likely to incline when becoming off balance, and portions on which pressure is likely to be applied. By assessing a care recipient using the device that outputs such precise and detailed information, it is possible to classify the care recipient according to factors such as a situation under which the care recipient is likely to fall down, how the care recipient becomes off balance, and the direction in which the care recipient falls down. By including the classification result in input data of the falling down determination processing, it is possible to perform processing in consideration of a target care recipient's falling down tendency, and thus possible to further improve processing precision. In addition, since these devices are used merely for classification of a care recipient and do not need to be used continuously, system construction is easy.

Note that, the method of this embodiment is not limited to machine learning. For example, the processing unit 310 may perform the falling down determination processing using different algorithms according to the classification result. Alternatively, the processing unit 310 may perform the falling down determination processing using different parameters (such as thresholds) according to the classification result. Besides, the method using the classification result for the falling down determination processing can be modified in various ways.

In addition, information used for the falling down determination processing is not limited to an output from the above assessment devices. For example, a user such as a caregiver may be able to input falling down history information representing the falling down history of a care recipient. For example, the user may input, using a device such as the caregiver terminal 400, information such as information identifying the care recipient, the timing when falling down has occurred, a location where falling down has occurred, and a situation under which falling down has occurred. The caregiver terminal 400 and the like sends, to the server system 300, the falling down history information thus input. The processing unit 310 obtains the risk of falling down by performing the falling down determination processing based on sensor information, flag information, and the falling down history information. The falling down history information may be used as input data of machine learning as in the case of the classification result described above, or may be used for processing other than machine learning. Alternatively, both the falling down history information and the classification result may be used for the falling down determination processing.

### <Forward Displacement Etc. in Wheelchair>

The foregoing description has been given of the falling down determination processing based on the sensor information of the wearable module 100 mounted on the chest and the like. However, the use of other sensors in combination therewith for the falling down determination processing is not precluded.

FIG. 18A illustrates an example of pressure sensors arranged in the wheelchair 520. In the example of FIG. 18A, four pressure sensors Se1 to Se4 are arranged on the back surface side of a cushion 521 disposed on the seat surface of the wheelchair 520. The pressure sensor Se1 is a sensor disposed on the front side, the pressure sensor Se2 is a sensor disposed on the rear side, the pressure sensor Se3 is a sensor disposed on the right side, and the pressure sensor Se4 is a sensor disposed on the left side. Note that, the front, rear, left, and right mentioned here indicate directions in a state where a care recipient sits on the wheelchair 520.

As illustrated in FIG. 18A, the pressure sensors Se1 to Se4 are connected to a control box 523. The control box 523 includes therein a processor that controls the pressure sensors Se1 to Se4 and a memory that serves as a work area of a processor. The processor mentioned here is a Micro Controller Unit (MCU) for example, but other processors may be used instead. The memory is an SRAM, DRAM, ROM, or the like. In addition, an external memory such as a USB memory may be connected to the control box 523. The control box 523 is housed in the pocket provided on the back surface of the wheelchair 520 as in the case of the communication device 200-2, for example.

The processor performs memory processing of detecting pressure values by operating the pressure sensors Se1 to Se4 and accumulating the detected pressure values in the memory (ROM). For example, the memory processing may be performed regularly, or alternatively the start/end of this processing may be controlled based on an operation by a caregiver. In addition, the control box 523 includes a communication module (not illustrated), and the processor may transmit, through this communication module, the pressure values thus accumulated to a device such as the communication device 200-2. For example, the processing unit 310 may perform the falling down determination processing (processing of detecting forward displacement and lateral displacement to be described later) based on the pressure values that are transmitted to the server system 300 through the communication device 200-2.

In addition, the processor may perform the falling down determination processing based on the pressure values. For example, the processor may activate the pressure sensors Se1 to Se4, reset various parameters, detect the pressure values, and execute the falling down determination processing and the memory processing. Alternatively, after the initialization is over, the processor may detect the pressure values and execute the falling down determination processing and the memory processing iteratively at predetermined intervals. This makes it possible to execute the falling down determination processing using the pressure sensors Se1 to Se4 without by way of the server system 300.

A care recipient sitting on the wheelchair 520 may feel pain on his/her buttocks and displace the position of his/her buttocks. For example, forward displacement indicates a state where his/her buttocks are displaced forward relative to normal, and lateral displacement indicates a state where his/her buttocks are displaced laterally relative to normal. In addition, there may be a case where the forward displacement and the lateral displacement occur at the same time and his/her center of gravity is displaced obliquely.

Although the forward displacement and the lateral displacement themselves are not equal to falling down, falling down is likely to occur under such situations, and thus they may become the risk of falling down. In that respect, by using the pressure sensors arranged on the cushion 521 as illustrated in FIG. 18A, it is possible to detect a change in the position of buttocks appropriately, and thus possible to detect the forward displacement and the lateral displacement precisely.

For example, assume that the timing when a care recipient moves to the wheelchair 520 and takes a normal posture is set at an initial state. In the initial state, since the care recipient sits deeply on the seat surface of the wheelchair 520, the value of the pressure sensor Se2 located rearward is supposed to be relatively large. On the other hand, if the forward displacement occurs, the position of his/her buttocks moves forward, and thus the value of the pressure sensor Se1 located forward increases. For example, the processing unit 310 may determine that the forward displacement occurs if the value of the pressure sensor Se1 increases by a predetermined amount or more compared to that of the initial state. Alternatively, instead of using the value of the pressure sensor Se1 by itself, processing may be performed using the relationship between the value of the pressure sensor Se2 and the value of the pressure sensor Se1. For example, a difference between voltage values which are outputs from the pressure sensor Se2 and the pressure sensor Se1 may be used, the ratio of the voltage values may be used, or the rate of change of the difference or ratio relative to that of the initial state may be used.

Likewise, if the lateral displacement occurs, the position of his/her buttocks moves leftward or rightward, and thus the value of the pressure sensor Se4 increases in the case of the leftward displacement and the value of the pressure sensor Se3 increases in the case of the rightward displacement. Accordingly, the processing unit 310 may determine that the leftward displacement occurs if the value of the pressure sensor Se4 increases by a predetermined amount or more compared to that of the initial state, and may determine that the rightward displacement occurs if the value of the pressure sensor Se3 increases by a predetermined amount or more compared to that of the initial state. Alternatively, the processing unit 310 may determine the rightward displacement or the leftward displacement using the relationship between the value of the pressure sensor Se4 and the value of the pressure sensor Se3. As in the example of the forward displacement, a difference between voltage values which are outputs from the pressure sensor Se4 and the pressure sensor Se3 may be used, the ratio of the voltage values may be used, or the rate of change of the difference or ratio relative to that of the initial state may be used.

Note that, as illustrated in FIG. 18A, the pressure sensor Se1 may be disposed at a position displaced to one of the left and right sides with respect to the center in the left-right direction of the seat surface, and the pressure sensor Se2 may be disposed at a position displaced to the other side with respect to the center in the left-right direction of the seat surface. The wheelchair 520 is foldable in many cases, and the seat surface may be made of a soft material that is foldable in the left-right direction. For example, as illustrated in FIG. 18A, the cushion 521 placed on the seat surface has a notch N in its back surface, and is foldable in the left-right direction at the notch N. In this case, for example, the pressure sensor Se1 is disposed rightward with respect to the notch N and the pressure sensor Se2 is disposed leftward with respect to the notch N. In this way, by displacing their positions in the left-right direction with respect to the center, it is possible to prevent the positions of the pressure sensors Se1 and Se2 and the notch N from overlapping with each other. As a result, the weight of a care recipient sitting on the cushion is transmitted to the pressure sensors Se1 and Se2 accurately, thus making it possible to precisely detect displacement in the front-rear direction.

In addition, as illustrated in FIG. 18A, the pressure sensors Se3 and Se4 may be arranged rearward with respect to the center in the front-rear direction of the cushion 521. From the perspective of reducing the chance of falling down from the wheelchair 520, it is preferable to make a care recipient sit deeply on the wheelchair 520. To put it differently, in a standard posture, the buttocks of the care recipient are located slightly rearward with respect to the center of the seat surface. In addition, when positional displacement occurs, the buttocks are assumed to move forward on the seat surface. Accordingly, by arranging the pressure sensors Se3 and Se4 at a position rearward of a standard position of the buttocks (in a narrow sense, an initial position of the buttocks), it is possible to inhibit the position of the buttocks and the position of the pressure sensors Se3 and Se4 from getting too close. As a result, even when a sensor whose maximum detectable pressure value is small is used, it is possible to inhibit its detected value from being saturated. For example, a small and thin sensor may be employed as the pressure sensors Se3 and Se4, which facilitates system construction.

Meanwhile, FIG. 18B is a diagram illustrating a cross-sectional structure of the cushion 521. As illustrated in FIG. 18B, when in use, the cushion 521 may have such a structure that a first layer 522a, a second layer 522b, and a third layer 522c are stacked in this order in a direction extending vertically from top to bottom. For example, the first layer 522a is a cushion provided independently of other layers, and the second layer 522b and the third layer 522c are cushions which are provided integrally and have the notch N formed in their lower surface. Here, the first layer 522a and the third layer 522c may be softer than the second layer 522b. Softness may be represented by the magnitude of a load applied, for example, when an object is depressed by a predetermined amount of deformation, such as Young's modulus. By providing the second layer 522b which is hard relative to the others, it is possible to disperse the weight of a care recipient, which makes the cushion comfortable to sit and suppresses bed sore. Meanwhile, by providing the first layer 522a and the third layer 522c, which are soft relative to the other, as layers to be in direct contact with the buttocks and the pressure sensors Se1 to Se4, it is possible to precisely detect pressure variation according to the position. Specifically, since a pressure value is likely to change largely when the position of the buttocks of a care recipient changes, processing precision can be improved.

Meanwhile, FIG. 18C is a diagram illustrating examples of a user interface unit 524 and a notification unit 525 provided in the control box 523. As illustrated in FIG. 18C, the user interface unit 524 includes: a power switch 524a; a recording start/end button 524b; and a determination button 524c. The notification unit 525 includes: a measurement in progress lamp 525a; a recording in progress lamp 525b; a forward displacement lamp 525c; and a lateral displacement lamp 525d. Although the forward displacement and the lateral displacement are described separately in this embodiment, this embodiment may be embodied in such a way that the forward displacement and the lateral displacement are collectively recognized as “displacement”, and the lamp 525c is lit when the processor detects no displacement (normal state) and the lamp 525d is lit when the processor detects the displacement.

For example, the power switch 524a is a switch for starting power supply to the processor, the memory, the pressure sensors Se1 to Se4, and the like. Once the power switch 524a is turned on, the above units transition to the operable state, and the pressure sensors Se1 to Se4 start measuring pressure values. The measurement in progress lamp 525a is lit during the measurement of pressure values. Once the measurement of pressure values is started, the processor performs the falling down determination processing based on the pressure values, and lights the forward displacement lamp 525c if determining that the forward displacement occurs and lights the lateral displacement lamp 525d if determining that the lateral displacement occurs.

The recording start/end button 524b is a button for controlling start/end of recording processing of storing pressure values, detected by the pressure sensors Se1 to Se4, in the memory. For example, when the recording start/end button 524b is pressed in a state where the measurement is in progress and no recording processing is started, the processor starts the processing of storing the pressure values in the memory. The recording in progress lamp 525b is lit while the recording processing is in progress. On the other hand, when the recording start/end button 524b is pressed while the recording processing is in progress, the processor ends recording the pressure values. The recording in progress lamp 525b is turned off once the recording processing ends.

The determination button 524c is a button for assigning flags to the pressure values while the measurement is in progress. For example, when the result of the falling down determination processing does not match the implicit knowledge of a caregiver (when it is determined from the result of the falling down determination processing that displacement occurs but the caregiver determines that no displacement occurs, and vice versa), the caregiver presses this determination button 524c. The processor assigns flags to the pressure values of the pressure sensors Se1 to Se4, acquired at the time of pressing the determination button 524c, and stores them in the memory. The processor performs processing of transmitting these pieces of data to the server system 300 through the communication device 200 and the like once the recording is over, and then the server system 300 updates the learned model based on the original learning data and the data on the pressure values assigned with the flags and transmits the updated learned model to the control box 523. The control box 523 communicates with the server system 300 at the timing when the power is turned on again to download the updated learned model. The control box 523 determines the forward displacement and the lateral displacement of a care recipient with the updated learned model and performs the falling down determination processing. Accordingly, it is possible to perform the falling down determination processing in a way that the caregiver thinks is optimum for each care recipient.

For example, in the method of this embodiment, the server system 300 may perform processing using learned model that acquires input data including pressure values corresponding to the pressure sensors Se1 to Se4 and outputs output data including accuracy on whether displacement occurs. Note that, the input data may be time series data that is a set of four pressure values acquired at multiple timings. The output data may be numeric data indicating the probability of an occurrence of displacement.

Alternatively, the output data may include both a probability value indicating the possibility of forward displacement and a probability value indicating the possibility of lateral displacement. As described above, by using the determination button 524c, a caregiver can point out an error in the result of presumption using the learned model. For example, if a flag is assigned in a situation where the learned model determines that displacement occurs and the forward displacement lamp 535c or the lateral displacement lamp 535d is lit, this flag is data indicating that “no displacement” is correct. In this case, data obtained by adding ground truth data, indicating that the probability of an occurrence of displacement is equal to 0 (or is sufficiently small), to the corresponding pressure value is transmitted to the server system 300 as training data for update. The same goes for the opposite case where, for example, if a flag is assigned in a situation where the learned model determines that “no displacement” occurs, data obtained by adding ground truth data, indicating that the probability of an occurrence of displacement is equal to 1 (or is sufficiently large), to the corresponding pressure value is transmitted to the server system 300 as the training data for update. This makes it possible to update the learned model appropriately. In this event, as described above, the learned model may be updated on a per-care recipient basis. For example, the learned model different on a per-care recipient basis may be used.

Note that, as described above, the falling down determination processing may be executed by the processor. In addition, response to a questionnaire such as the attributes of a care recipient may be input through a mobile information terminal that is capable of communication with the control box 523. The response to the questionnaire is sent to the server system 300 and used for updating the learned model. Based on the result of the questionnaire, the server system 300 classifies care recipients into classes, and updates the learned model on a per-class basis. This enables the control box to perform the optimum falling down determination processing for care recipients having the same attributes. For example, the learned model different for each of the attributes described above may be used, or the input data may be added with data indicating the attributes of a care recipient. Here, the attributes of a care recipient may include information such as the sex, age, physique, body conditions, motion, and communication. For example, the physique may be information indicating any of the thin body type, normal body type, and fat body type, or may be a numeric value such as BMI. The body conditions are information identifying whether a care recipient has a hemiplegia and its location, whether the care recipient has a pain and its location, and deformation of the spinal cord (such as whether the care recipient has a hump back or scoliosis and its intensity). The motion is information indicating recipient can sit back by himself/herself. The communication is information indicating whether a target care recipient can communicate with others.

Note that, although one determination button 524c is provided in this embodiment, the embodiment is not limited to this, and multiple buttons may be provided for separately recording two events, i.e. forward displacement and lateral displacement, for example. For example, in order to assign ground truth data in a case where the learned model has a configuration of outputting the possibility of forward displacement and the possibility of lateral displacement individually, it is preferable to be able to input whether displacement that currently occurs is forward displacement, lateral displacement, or both. In this case, flags can be assigned appropriately by providing a button for assigning a forward displacement flag and a button for assigning a lateral displacement flag. In addition, since flags to be assigned by a user such as a caregiver are not limited to information identifying the type of displacement (such as no displacement, forward displacement, and lateral displacement), the number of determination buttons 524c may be expanded to three or more. For example, a caregiver may be able to input a flag for identifying a part of data on a series of pressure values recorded by the recording processing, corresponding to a partial period of time, using the determination button 524c. For example, a caregiver may determine that, among data acquired for a period from the start to end of the recording processing using the recording start/end button 524b, a part of the data corresponding to a partial period of time is characteristic data (e.g. corresponds to a period during which displacement occurs). In this case, the caregiver can assign a period flag to the part of data by inputting the start/end of the corresponding period using the determination button 524c. For example, it is possible to extract, among pressure values included in one file recorded by the recording processing, only pressure values assigned with the period flag and use them for the processing of updating the learned model. In addition, flags that a user can assign are not limited to the type of displacement and period of time, and various modifications are possible. Note that, expansion contents of the determination button 524c are not limited to an increase in the number of buttons. For example, an interface other than a button may be used, or inputs different according to the number of pressing the button and a period during which the button is pressed may be made. In other words, any determination button 524c will do as long as it is an interface capable of accepting inputs according to the type of flags used, and its specific aspects can be modified in various ways.

In addition, in the detection of forward displacement and lateral displacement, a mobile terminal device such as a smartphone may be used as a sensor. For example, a smartphone including an acceleration sensor is widely used. For example, the falling down determination processing may be performed by fixing the smartphone on the back surface of the seat surface using a fastening tool such as a band.

As described previously, the seat surface of the wheelchair 520 may be made of a soft material that is foldable. In a case where the seat surface is soft, a portion where a care recipient puts his/her buttocks on is depressed deeply and the other portion is lifted up relative to that portion, and therefore the angle of the seat surface is considered to change largely according to the posture of the care recipient. For example, if forward displacement occurs, the front side of the seat surface is depressed more than the case of the normal state as a reference state. In this case, since the posture of a smartphone fixed on the back surface also changes together with the change of the seat surface, it is possible to detect the forward displacement appropriately using the acceleration sensor.

Likewise, if lateral displacement occurs, one of left and right sides of the seat surface is depressed deeply and the other side is lifted up relative to that side. In this case, since the posture of the smartphone also changes in accordance with the displacement direction, it is possible to detect the lateral displacement appropriately using the acceleration sensor of this smartphone.

### <Modified Example Related to Walking>

The foregoing description has been given of the example where the communication device 200 corresponding to the case of during walking is provided in addition to the communication devices 200-1 to 200-6 of FIG. 2, for example, and target sensor information is determined as data indicating during walking when it is associated with the identification information of this communication device 200. However, since the sensor information indicating during walking has characteristics different from the characteristics of the bed 510, the wheelchair 520, and the toilet 600, the processing unit 310 may determine whether the sensor information corresponds to the case of during walking based on the characteristics.

Specifically, during walking, a care recipient needs to repeat stepping out his/her right and left legs alternately in contrast to the other locations. As a result, the upper body of the care recipient sways right and left with two steps as one cycle of walking. Accordingly, an acceleration value of the axis corresponding to the left-right direction of the care recipient becomes periodic data. In the above example, the axis corresponding to the left-right direction is the y axis.

For example, the processing unit 310 determines whether a care recipient is during walking by determining the periodicity in the acceleration value of the y axis. As an example, the processing unit 310 detects the upper peak or lower peak of the acceleration value in the y axis and obtains a peak interval. The upper peak indicates a point where the value goes from increasing to decreasing, and the lower peak indicates a point where the value goes from decreasing to increasing. The peak interval indicates a time difference between a given peak and the next peak. For example, the processing unit 310 obtains variation in the peak interval during a predetermined period of time, and determines that the peak interval has high periodicity and thus a care recipient is walking if this variation is equal to or smaller than a predetermined value.

Note that, the processing of determining periodicity is not limited to this. For example, an interval between zero crossover points may be used instead of the peak. The zero crossover point indicates a point where the value goes from positive to negative or a point where the value goes from negative to positive. Alternatively, the processing unit 310 may perform frequency transform such as fast Fourier transform (FFT) and determines the periodicity based on a distribution after the transform. For example, the processing unit 310 determines that a care recipient is walking if determining that variation in frequency is equal to or smaller than a predetermined value based on a factor such as the peak width at half height of a frequency-axis waveform.

Meanwhile, the foregoing description has been given of the falling down determination processing using machine learning such as NN; however, in the falling down determination processing in the case of walking, determination may be made based on the periodic signal described above. For example, the processing unit 310 may determine that the risk of falling down is high if the periodicity decreases compared to that of the normal state. This is because, when the periodicity decreases, it means that a care recipient has lost the rhythm of stepping out his/her right and left legs and is suspected to have encountered an event such as failing to move his/her legs forward properly or stumbling over something.

In addition, the experiment by the applicant has shown that, prior to and after falling down, although data has periodicity in the acceleration value of the y axis, the data is different from that of the normal state in that the amplitude and cycle length vary or the lower peak value and upper peak value of the acceleration value vary, for example. Accordingly, in the case of during walking, the processing unit 310 may obtain a parameter such as the amplitude and cycle length described above, and determine the risk of falling down based on variation of the parameter. For example, the processing unit 310 may classify cases of losing the periodicity into several patterns and determine falling down based on whether a case in question corresponds to any of these patterns. Note that, the classification into patterns in the case of during walking will be described in processing of presuming walking ability to be described later.

2.3 Collaboration with Peripheral Device

As described above using the figures such as FIG. 5, the foregoing description has been given of the example of giving notification to a caregiver if the risk of falling down is detected. However, the processing unit 23 of the information processing apparatus 20 may execute other processing based on the falling down determination processing. Note that, as in the above example, the following description will be given of an example where the information processing apparatus 20 is the server system 300.

For example, the processing unit 310 may control the peripheral device 700 based on the falling down determination processing. This control may be triggered by detection of the risk of falling down of a care recipient based on the falling down determination processing, for example. The peripheral device 700 mentioned here indicates a device that is used by a care recipient and disposed near the care recipient in the care recipient's daily life. Thus, by collaboration with the peripheral device 700, it is possible to inhibit a care recipient from falling down or, even if falling down cannot be inhibited, possible to ease impact by the falling down.

FIGS. 19A and 19B are diagrams illustrating a table 530 which is an example of the peripheral device 700. For example, the table having a compact operation mechanism is stated in Japanese Patent Application No. 2015/229220, filed on Nov. 24, 2015, and entitled “OPERATION MECHANISM AND MOBILE TABLE INCLUDING THE SAME”. This patent application is incorporated herein in its entirety by reference.

FIGS. 19C and 19D are diagrams illustrating the wheeled walker 540 which is an example of the peripheral device 700. For example, the wheeled walker designed for improvement in its weight reduction, stability, and maintenance is stated in Japanese Patent Application No. 2005/192860, filed on Jun. 30, 2005, and entitled “WALKING AID”. This patent application is incorporated herein in its entirety by reference.

The table 530 is a mobile table including casters Ca11 to Ca14, for example. In addition, the wheeled walker 540 is a device for aiding walking of a care recipient and includes casters Ca21 to Ca24, for example. The table 530 has a function of limiting its movement by locking at least a part of the casters Ca11 to Ca14. For example, Japanese Patent Application No. 2015/229220 discloses a brake mechanism, an operation wire that transmits a motion to the brake mechanism, and the like. Likewise, the wheeled walker 540 has a function of limiting its movement by locking at least a part of the casters Ca21 to Ca24. For example, a wheeled walker has been known which has a brake lever near a grip part to be gripped by a care recipient and is braked using a wire when the care recipient grips the brake lever. The wheeled walker 540 may have a function of keeping the brake mechanism in the braking state, or may have a lock mechanism that uses a wire different from the wire that operates in conjunction with the brake lever.

However, the peripheral device 700 is not always locked. For example, the mobile table disclosed in Japanese Patent Application No. 2015/229220 is a table that is braked in its normal state and has an unlock function of releasing the brake when operation levers 531 are operated. For example, in FIG. 19A, the brake is released by moving the two operation levers 531 upward. However, in some tables with the unlock function, the unlock is kept released. For example, in a case where a caregiver moves the table 530, the caregiver may use a lock lever to keep the operation levers 531 in the operation state instead of keeping operating the operation levers 531 manually. In this case, the brake is released. It will not pose a problem since the unlock function works once the caregiver turns the lock lever back after moving the table 530. For example, the table 530 may have such a configuration that the lock lever is turned back by further operating (e.g. by moving further upward) the operation levers 531 kept in the operation state. However, the unlock may be kept released by leaving the lock lever unattended due to human error.

Meanwhile, in the case of the wheeled walker 540, the risk of falling down occurs when a care recipient loses a balance while walking using the wheeled walker 540, for example. In this case, as described previously, the care recipient can lock the casters Ca21 to Ca24 if he/she can operate the brake levers. However, it is not easy for the care recipient who is about to fall down to pull the brake lever properly, so that the lock mechanism may fail to function. In addition, the wheeled walker 540 may have such a configuration that the lock mechanism is provided only in the vicinity of the casters and no brake lever exists, like a brake 547 which will be described later using FIG. 19D. In this case, it is hard for the care recipient who is about to fall down to quickly lock the casters using his/her feet.

Accordingly, when the risk of falling down is detected, the processing unit 310 may perform control to lock the casters of the peripheral device 700 that i capable of moving by the casters. The peripheral device 700 that is capable of moving by the casters is the table 530 or the wheeled walker 540, for example, but another peripheral device 700 may be used instead. For example, the bed 510 (including child's bed) with casters that has a function of electrically locking the casters has been known. The peripheral device 700 of this embodiment may include the bed 510 as described above. When a care recipient is about to fall down, normally, the care recipient grips the peripheral device 700 in many cases. According to the method of this embodiment, it is possible to set the peripheral device 700, which the care recipient is about to grip, in the lock state reliably. Since the movement of the peripheral device 700 with respect to the floor surface is restricted in the lock state, it is possible to support the body of the care recipient appropriately and thus prevent the care recipient from falling down.

Alternatively, the peripheral device 700 may be a device having a height adjustment function. The peripheral device having a height adjustment function may be the bed 510 illustrated in FIG. 19E, for example. The bed 510 mentioned here is a mobile bed capable of changing the height of sections. However, other devices may be used as the peripheral device 700 having a height adjustment function.

If the risk of falling down is detected, the processing unit 310 may perform control to lower the height of the peripheral device 700. The angle and height of the sections of the bed 510 are adjusted depending on situations such as the case of sitting up when standing up or moving to the wheelchair 520, the case of taking a meal on the bed 510, and the case of changing a diaper. However, when the sections are located at a high position, the height of the mattress placed on the sections and the height of side rails provided on side surfaces are also high. Accordingly, it is sometimes hard for a care recipient who is about to fall down to grip the mattress and hand rails or fall down onto the mattress safely. In that respect, according to the method of this embodiment, since the height of the bed 510 can be lowered down when there is a risk of falling down, the care recipient can be appropriately inhibited from getting injured due to falling down.

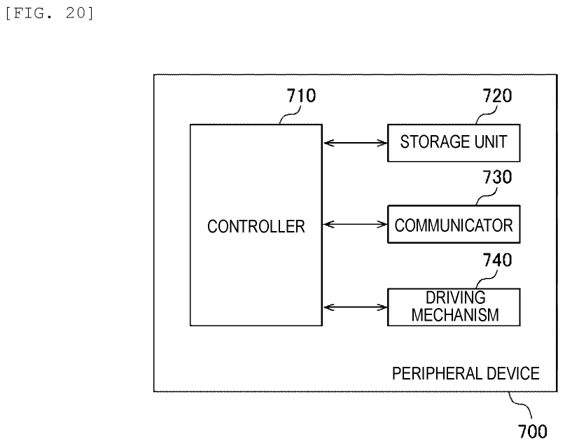

FIG. 20 is a diagram illustrating the configuration of the peripheral device 700. The peripheral device 700 includes: a controller 710; a storage unit 720; a communicator 730; and a driving mechanism 740.

The controller 710 is configured to control various parts of the peripheral device 700. The controller 710 may be a processor. Various processors such as a CPU, a GPU, and a DSP can be used for the processor mentioned here. The controller 710 of this embodiment corresponds to a processor in a substrate box 533 which will be described later and a processor in a housing 542 or a second housing, for example.

The storage unit 720 is a work area of the controller 710, and is implemented by various memories such as SRAM, DRAM, and ROM. The storage unit 720 of this embodiment corresponds to a memory in the substrate box 533 which will be described later and a memory in the housing 542 or the second housing, for example.

The communicator 730 is an interface for performing communication via a network, and includes an antenna, an RF circuit, and a baseband circuit, for example. The communicator 730 may be operated under control of the controller 710 or may include a processor for communication control that is different from the controller 710. The communicator 730 may communicate with the server system 300 communication using LAN, by wireless for example.

Alternatively, as in the example of the bed 510 and the like of FIG. 2, the communication device 200 may be fixed on the peripheral device 700 using a holder, for example. In this case, the communication device 200 may communicate with the server system 300. The communicator 730 acquires information from the processing unit 310 by communicating with the communication device 200 by any method such as Bluetooth.

The driving mechanism 740 has a mechanical configuration for operating the peripheral device 700. For example, the driving mechanism 740 may be a solenoid 534. As illustrated in FIGS. 19A and 19B, the table 530 includes the pair of operation levers 531 and a fixing member 532 that fixes the driving mechanism 740 on the table 530. The fixing member 532 includes: a major surface 532a that has a relatively large area; a surface 532b that intersects with the major surface 532a and is parallel with a table surface; and a surface 532c that intersects with the major surface 532a and is parallel with one surface of a support part, and is fixed on the table 530 using these surfaces. Note that, being parallel mentioned here includes being substantially parallel, and includes a surface that is at an angle of a predetermined value or smaller with its symmetric surface (e.g. the table surface in the above example). Various methods such as screwing and bonding can be used as the fixing method. In addition, as illustrated in FIG. 19B, the fixing member is provided with the solenoid 534 and the substrate box 533 that houses therein a substrate for driving the solenoid 534. The substrate mentioned here is a substrate on which a processor for controlling the solenoid 534 and a memory are mounted, for example.

As illustrated in FIG. 19A, in a state where the fixing member 532 is fixed on the table 530, the solenoid 534 is disposed below any one of the pair of operation levers 531. More specifically, the solenoid 534 is disposed at such a position that its movable core bumps against the operation lever 531 when the movable core moves in response to driving of a processor in the substrate box 533. For example, in a case where the processing unit 310 outputs a control signal instructing locking of the table 530, this control signal is transmitted to the substrate via the communication device 200 provided in the table 530, and the substrate drives the solenoid 534 based on the control signal. By doing so, the operation of moving the operation lever 531 upward is performed based on the control signal from the processing unit 310 and thus the fixing of the operation lever 531 is released, so that the table 530 shifts to a state where the unlock function works.

The driving mechanism 740 may also include a wire 546 for operating the brake mechanism and a motor 545 that rolls up the wire. As illustrated in FIGS. 19C and 19D, the wheeled walker 540 includes: a base frame; a support that stands on the base frame; an adjustment support that is provided on the support so as to be expandable and contractible; and a leaning part that is provided on an apex part of the adjustment support and designed to support the upper body of the user. The base frame includes: a linear lateral pipe 541a; a pair of longitudinal pipes 541b that are integrally coupled, on their one end sides, to the vicinity of both ends of the lateral pipe 541a respectively and that are expanded, on their other end sides, more than the clearance between their one end sides; and a base portion frame member 541c that integrally couples the pair of longitudinal pipes 541b to each other and is designed to attach the support thereon. The driving mechanism 740 in this case may be housed in the housing 542. The housing 542 includes hook parts 543 and 544, and is held so as to be hung on one of the pair of longitudinal pipes 541b by the hook parts 543 and 544. As illustrated in FIG. 19D, a motor 545 is provided inside the housing, and the motor 545 is configured to roll up and release a wire 546. Note that, although not illustrated in FIG. 19D, a processor that drives the motor 545 and a memory that serves as a work area of the processor may be mounted inside the housing 542.

As illustrated in FIG. 19C, the caster Ca23 is provided with the brake 547. The brake 547 includes a plate-shaped member, for example, and the caster Ca23 is locked by pulling up the plate-shaped member. The wire 546 described above is coupled to the plate-shaped member of the brake 547. Thus, in response to an event where the motor 545 rolls up the wire 546, the plate-shaped member moves upward to lock the caster Ca23. On the other hand, in response to an event where the motor 545 rolls back the wire 546, the plate-shaped member moves downward to release the lock of the caster Ca23.

Note that, the configuration of the driving mechanism 740 of the wheeled walker 540 is not limited to this. For example, the second housing for housing the processor and the memory therein may be provided in addition to the housing 542. The second housing may be fixed on the base portion frame member 541c, for example. The motor 545 of the housing 542 and the processor of the second housing are electrically connected to each other using a signal line. In addition, although the mechanism for locking the caster Ca23 is described with reference to FIG. 19C, a caster to be locked may be other than this caster. Further, two or more casters out of the casters Ca21 to Ca24 may be set as casters to be locked. For example, the housing 542 may be provided in the vicinity of each of the casters Ca23 and Ca24, and both of them may be connected to the second housing provided in the base portion frame member 541c.

Meanwhile, the driving mechanism 740 may include various mechanisms for changing the height of the sections of the bed 510. For example, the driving mechanism 740 may be a mechanism for lowering the height of the sections by driving leg parts of the bed 510 while keeping the angle of the sections.

FIG. 21 illustrates a configuration example of the information processing system 10 including the peripheral device 700. The wearable module 100 and the communication device 200 are the same as those in the example of FIG. 5. The server system 300 is also the same as that in the above example in that information associating sensor information from the communication device 200 with the identification information of the communication device 200 is acquired to perform the falling down determination processing according to the location.

In the example of FIG. 21, the table 530, the wheeled walker 540, and the bed 510 illustrated in FIGS. 19A to 19E are illustrated as the peripheral device 700; however, the peripheral device 700 may include other devices that are movable by casters and may include other devices that are capable of adjusting their height.

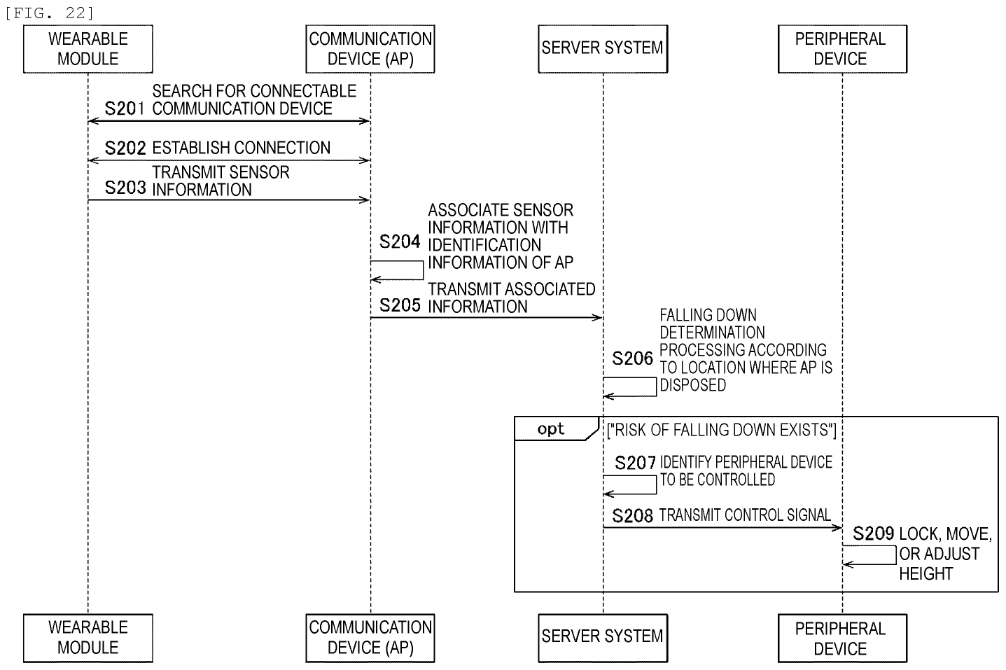

FIG. 22 is a sequence diagram illustrating processing in the system illustrated in FIG. 21. First, at Step S201, the wearable module 100 determines whether the connectable communication device 200 exists nearby. At Step S202, connection between the wearable module 100 and the communication device 200 is established.

At Step S203, the wearable module 100 transmits sensor information, detected by the acceleration sensor 120, to the communication device 200 using the communication module 130. At Step S204, the communication device 200 performs processing of associating the sensor information received at Step S203 with the identification information of the communication device 200. At Step S205, the communication device transmits the associated information to the server system 300. At Step S206, based on the received information, the server system 300 executes the falling down determination processing according to the location of a care recipient. The processing illustrated in Steps S201 to 206 is the same as that of Steps S101 to S106 in FIG. 9.

If determining that the risk of falling down exists, at Step S207, the server system performs processing of identifying the peripheral device 700, which is located near a care recipient associated with the wearable module 100, based on at least one of the location where the communication device 200 is disposed, which is identified by location information, and information identifying the care recipient.

As described above, in this embodiment, control to cause a device, which a care recipient who is about to fall down quickly tries to grip, to shift to a state appropriate for preventing falling down is performed. Accordingly, to control the peripheral device 700 that is located at a position where a care recipient cannot easily grip is not supposed to be helpful in terms of preventing an injury etc. due to falling down. Further, to drive the peripheral device 700 which is being used by other care recipients or caregivers is rather risky and impairs convenience. To deal with this, while multiple peripheral devices 700 are assumed to be used in a nursing care facility and the like, it is necessary to appropriately determine which of these devices is to be controlled.

For example, as described in the falling down determination processing above, the processing unit 310 identifies the location where the communication device 200 is disposed based on the identification information of the communication device 200 associated with sensor information. The processing unit 310 may identify the peripheral device 700, which is disposed at the identified location, as a device to be controlled. For example, the server system 300 may store peripheral device information obtained by associating the peripheral device 700 with the location where this peripheral device is disposed. On the basis of the location identified based on the identification information of the communication device 200 and the peripheral device information, the processing unit 310 identifies the peripheral device 700, which is located near a care recipient who has the risk of falling down, as a device to be controlled. Note that, location information included in the peripheral device information may be information registered by a user such as a caregiver, or may be information dynamically changed by tracking processing using sensors.

Alternatively, as illustrated in FIG. 8B, the processing unit 310 can identify a care recipient, associated with the wearable module 100, based on module information. Meanwhile, the server system 300 may store peripheral device information obtained by associating the peripheral device 700 with a care recipient who is a user of this peripheral device 700. For example, in a nursing care facility, since a schedule on what kind of assistance is to be provided to which care recipient and at what time has been determined already, it is supposed to be identifiable when and by which care recipient the peripheral device 700 such as the wheeled walker 540 is to be used. In addition, since the bed 510 is highly probably occupied by one care recipient, it is easy to associate the peripheral device 700 with a care recipient who is a user of this peripheral device. Accordingly, by identifying a care recipient who has the risk of falling down based on the identification information of the wearable module 100 from which sensor information is transmitted, it is possible to identify the peripheral device 700 which is highly probably used by this care recipient.

At Step S208, the processing unit 310 performs processing of transmitting a control signal to the peripheral device 700 thus identified. At Step S209, the controller 710 of the peripheral device 700 operates the driving mechanism 740 according to the control signal. Note that, the control signal transmitted at Step S208 may be a signal instructing locking or a signal giving instructions to lower the height of the sections. Alternatively, the control signal may be a signal indicating that the risk of falling down exists, and specific control contents may be determined by the controller 710 of the peripheral device 700.

Note that, the foregoing description has been given of the example where, in the peripheral device 700 that is capable of moving by the casters, the casters are locked based on the risk of falling down. However, the method of this embodiment is not limited to this.

As described above, the method of this embodiment prevents an injury etc. due to falling down by causing a care recipient who is about to fall down to grip the stable peripheral device 700. For this reason, it is important that the distance between the care recipient and the peripheral device 700 is near enough to enable the care recipient to quickly grip the peripheral device.

Accordingly, when the risk of falling down is detected, the processing unit 310 may perform control to move the peripheral device 700 closer to the care recipient by driving the casters of the peripheral device 700. By doing so, the distance between the peripheral device 700 and the care recipient gets closer and therefore the care recipient can easily grip the peripheral device 700, thus making it possible to further suppress an influence due to falling down.

For example, since the wearable module 100 of this embodiment has the acceleration sensor 120, it can perform autonomous positioning based on sensor information of the acceleration sensor 120. Note that, positioning by the wearable module 100 may be executed by the communication device 200 or the server system 300. In particular, since the position of the communication device 200 is known, the position of a care recipient can be presumed by correcting the autonomous positioning result using information such as whether there is communication with the communication device 200 and the intensity of radio wave received during communication.

Meanwhile, as described previously, devices such as a smartphone corresponding to the communication device 200 may be arranged in the peripheral device 700. Since these devices include an acceleration sensor, they can perform autonomous positioning as in the case of the wearable module 100. In addition, the position of a care recipient can be presumed by correcting the autonomous positioning result using information such as a communication status with other communication devices 200 and information on the use and management of equipment in a nursing care facility etc. The use and management information may include, for example, information such as information on when and where the identified wheeled walker 540 is to be used and information on where it is stored while not in use.

In this way, the position of a care recipient and the position of the peripheral device 700 can be presumed. Although the example of the autonomous positioning based on the acceleration sensor has been described above, the position may be presumed by other methods such as image processing using an image taken by a camera disposed inside a nursing care facility and three-point positioning using BLE beacons.

Based on the position information thus presumed, the processing unit 310 of the server system 300 identifies positional relationship between a care recipient who is about to fall down and the peripheral device 700 located near the care recipient. For example, the processing unit 310 presumes a movement direction and the amount of movement of the peripheral device 700 for moving it closer to the care recipient, and determines the driving amount of the casters of the peripheral device 700 based on the presumption result. More specifically, the processing unit 310 may perform processing of determining the amount of rotation of the motor that drives the casters. The processing unit 310 notifies the peripheral device 700 of the amount of rotation thus determined, and the controller 710 of the peripheral device 700 performs control to drive the motor by this amount of rotation. In addition, a part of the processing by the server system 300 may be executed by the peripheral device 700 or by the communication device 200 disposed in the peripheral device 700.

Further, after performing control to move the peripheral device 700 to a position within a predetermined distance or smaller from the care recipient, the processing unit 310 may perform control to lock this peripheral device 700. By doing so, since the peripheral device 700 thus controlled is located at a position where the care recipient can easily grip and is set in the lock state, it is possible to appropriately suppress an influence due to falling down of the care recipient.

Further, the peripheral device 700 is not limited to the bed 510, the table 530, and the wheeled walker 540, and may be other devices. For example, the peripheral device 700 may include an airbag to be worn by the care recipient. The airbag is a device that is mounted on the waist or the like of the care recipient in a contracted state, for example, and is a device that automatically expands upon receipt of a control signal. For example, the airbag includes a communication module that communicates with the communication device 200 and a processor such as a microcomputer.

When the risk of falling down of a care recipient is detected, the processing unit 310 outputs, to the airbag which is worn by this care recipient, a control signal instructing expansion of the airbag. The control signal is transmitted to the processor of the airbag via the communication device 200, for example. The processor of the airbag executes control to expand the airbag based on this control signal. By doing so, it is possible to prevent an occurrence of an injury due to falling down by identifying a care recipient who has the risk of falling down and activating the airbag of this care recipient.

Further, the peripheral device 700 of this embodiment may be an airbag that is disposed on a wall surface or a floor surface of the toilet 600. When the risk of falling down of a care recipient in the toilet 600 is detected, the processing unit 310 may output, to the airbag which is disposed in the toilet 600, a control signal instructing expansion of the airbag. This makes it possible to prevent an occurrence of an injury due to falling down. The toilet 600 is particularly narrow in area compared to the living room and the dining room, for example, so that it is easy to narrow down the position of the wall surface or the floor surface against which a care recipient may hit his/her body hard at the time of falling down. Accordingly, by disposing the airbag in advance and expanding it in accordance with the risk of falling down, it is possible to appropriately prevent an occurrence of an injury. However, an option of disposing the airbag at a location other than the toilet 600 is not precluded.

In addition, in this embodiment, when the risk of falling down is detected, notification may be given to the caregiver terminal 400 as described above using FIG. 5, control over the peripheral device 700 may be performed as described above using FIG. 21, or both of them may be performed. Alternatively, which of them is performed may be switched according to the result of the falling down determination processing.

For example, information identifying the length of time before falling down may be output as output data of the falling down determination processing. As an example, as will be described later in relation to the description of walking ability, sensor information of the acceleration sensor 120 may be classified into patterns in the falling down determination processing in the case of walking (including processing of presuming walking ability). For example, the storage unit 320 may hold a table that associates a pattern with the length of time before falling down, and the processing unit 310 may determine the length of time before falling down based on the pattern classification result and this table.

Then, the processing unit 310 may control the peripheral device 700 if the length of time before falling down is equal to or smaller than a predetermined threshold, and give notification to the caregiver terminal 400 if the length of time before falling down is larger than the threshold. The control over the peripheral device 700 is control to activate the airbag, for example. In a case where the length of time before falling down is short, even if notification is given to the caregiver terminal 400, a caregiver may not be able to intervene properly. For example, it is conceivable that the caregiver is unable to support the care recipient promptly due to reasons that the caregiver is not in the vicinity of a care recipient or that the caregiver is currently providing assistance to another care recipient. In that respect, since the airbag can be activated in a short period of time, it is possible to prevent an occurrence of an injury appropriately. Meanwhile, in a case where there is enough length of time before falling down, by prioritizing the caregiver's intervention, it is possible to reduce the cost for exchanging the airbag etc.

### 3. Specific Example of Processing According to Location

Note that, in the foregoing description, the falling down determination processing has been described as an example of processing according to the location. However, the processing executed at each location is not limited to this. Hereinbelow, a description will be given of a method of appropriately using implicit knowledge in specific situations such as taking a meal, adjusting the position on the bed 510 and the wheelchair 520, and changing a diaper.

Note that, in each processing to be described below, the result of identification of the location of a care recipient based on location information may be used as at least one of triggers as described previously. For example, the processing unit 310 identifies the location of a care recipient based on the location information, and executes control to activate a sensor disposed at this location. Then, based on information from the sensor thus activated, the processor executes each processing to be described below. Specifically, in a case where a care recipient is on the wheelchair 520, the processing unit 310 activates the seat surface sensors (pressure sensors Se1 to Se4) illustrated in FIG. 18. Meanwhile, in a case where a care recipient is on the bed 510, the processing unit 310 activates devices such as a detection device 810 that detects heartbeat, respiration, body motion, and the like to be described later using FIG. 33 to start processing related to bed departure and sleeping. Meanwhile, in a case where a care recipient is in the toilet 600, the processing unit 310 may activate a pressure sensor and the like disposed on the floor of the toilet 600. Meanwhile, the processing unit 310 is not limited to one that activates all sensors arranged at a target location. For example, the processing unit 310 may select a sensor to be activated according to the attributes of a target care recipient. This makes it possible to activate a necessary sensor appropriately based on location information.

However, the method of this embodiment is not limited to this, and the location and situation may be identified by another method and each processing to be described below may be started based on this identification result. In other words, in each processing to be described below, the processing of identifying the location based on location information is not essential.

### 3.1 Taking Meal

For example, a care recipient who uses the wheelchair 520 moves from the bed 510 to the wheelchair 520 in the living room etc., then moves to the dining room with this wheelchair 520, and then starts taking a meal while sitting at the table. Accordingly, in this embodiment, each processing to be described later may be executed if the wearable module 100 transmits sensor information to the communication device 200-5 that is disposed in the dining room. Alternatively, in a case where the communication device 200-5 is omitted, each processing to be described later may be executed if the wearable module 100 transmits sensor information to t device 200-2 corresponding to the wheelchair 520 and if it is determined that the wheelchair 520 is located at a location for taking a meal such as the dining room. Note that, the position of the wheelchair 520 may be determined by autonomous positioning using the acceleration sensor. Alternatively, whether the care recipient is at the location for taking a meal may be determined by recognizing the care recipient by another sensor such as a camera disposed in the dining room and the like. In addition, the following processing may be triggered by other conditions such as an event of pressing a start button displayed on the mobile terminal device 410 of a caregiver.

FIG. 23 is a diagram illustrating implicit knowledge in taking a meal. FIG. 23 wholly illustrates implicit knowledge in taking a meal, and the implicit knowledge is classified into the eating pattern, the thickness (concentration), and the eating assistance. The eating pattern corresponds to implicit knowledge for adjusting an eating pattern such as a size into which cooking ingredients are cut. The thickness (concentration) corresponds to implicit knowledge for adjusting the degree of thickness of a meal. The eating assistance corresponds to implicit knowledge for supporting a care recipient in taking a meal.

In FIG. 23, the “situation” indicates the situation of a care recipient, and the action indicates an action that should be executed by a caregiver in the case of this situation. For example, based on his/her own experience, a skilled worker determines whether a care recipient is in a situation of “no longer able to bite off food” and, if this situation applies, takes measures such as “providing the food by cutting it into small pieces on site”, “stopping the meal”, and “seeing a dentist for eating guidance”. In other words, the implicit knowledge of the skilled worker may be information associating the situation of the care recipient with an action that should be executed in this situation.

In a case where multiple actions are associated with one situation as in FIG. 23, the priority may be given to each action. For example, in the case of the implicit knowledge corresponding to the above example, in the situation of “no longer able to bite off food”, “providing the food by cutting it into small pieces on site” is prioritized and, if this does not solve the problem, “stopping the meal” is executed. In addition, at a different timing after the meal, the eating function is tried to be recovered by “seeing a dentist for eating guidance”. Such a series of actions according to these situations are preferable actions to be executed by a skilled worker, and the method of this embodiment provides support to a caregiver so that the caregiver can execute the same actions as a skilled worker irrespective of the degree of proficiency of the caregiver. Note that, the actions illustrated in FIG. 23 are an example of actions to be executed according to the situations, and other actions may be added. For example, for the situation of “no longer able to bite off food”, actions such as “reconsidering the contents of meal” and “adjusting the volume of meal” may be added. To put it differently, the actions in this embodiment may include actions for making the situation of “no longer able to bite off food” better when this situation occurs and actions for making the situation of “no longer able to bite off food” less likely to happen at timings after this situation occurs. In this respect, the same goes for other situations.

A skilled caregiver can determine, by simply observing the appearance of a care recipient, whether the care recipient is in the situations illustrated in FIG. 23 such as the situation of “no longer able to bite off food”. However, in order to make even a beginner etc. provide assistance according to the situations, it is necessary to automatically detect the situation of a care recipient using a device including a sensor. Note that, as illustrated in FIG. 23, implicit knowledge may include the attributes of a user. This indicates to which care recipient with what kind of attributes the target implicit knowledge can be employed. Thus, according to the method of this embodiment, the attributes of a care recipient may be determined, and whether each situation should be automatically detected may be switched based on the attributes.

FIG. 24 is a diagram illustrating devices used in a scene of taking a meal. As illustrated in FIG. 24, a throat microphone TM mounted around the neck of a care recipient and the communication device 200-5 having a camera are used as the devices. Note that, another terminal device having a camera may be used instead of the communication device 200-5. The throat microphone TM is configured to output audio data generated by swallowing, coughing, and the like of a care recipient. The camera of the communication device 200-5 is configured to output an image in which how a care recipient is taking a meal is taken. For example, the communication device 200-5 is a smartphone or the like that is placed on a table where a care recipient is taking a meal. In addition, as described above using FIG. 2, the wearable module 100 is mounted on the chest or the like of a care recipient.

The audio data of the throat microphone TM and the image taken by the communication device 200-5 are transmitted to the server system 300. For example, the communication device 200-5 acquires the audio data from the throat microphone TM using Bluetooth or the like, and transmits this audio data and the image taken by the camera to the server system 300. Note that, the audio data and the taken image may be transmitted to the server system 300 via the communication device 200-2 disposed in the wheelchair 520. Besides, various modifications are possible as the method of transmitting the output of each device to the server system 300.

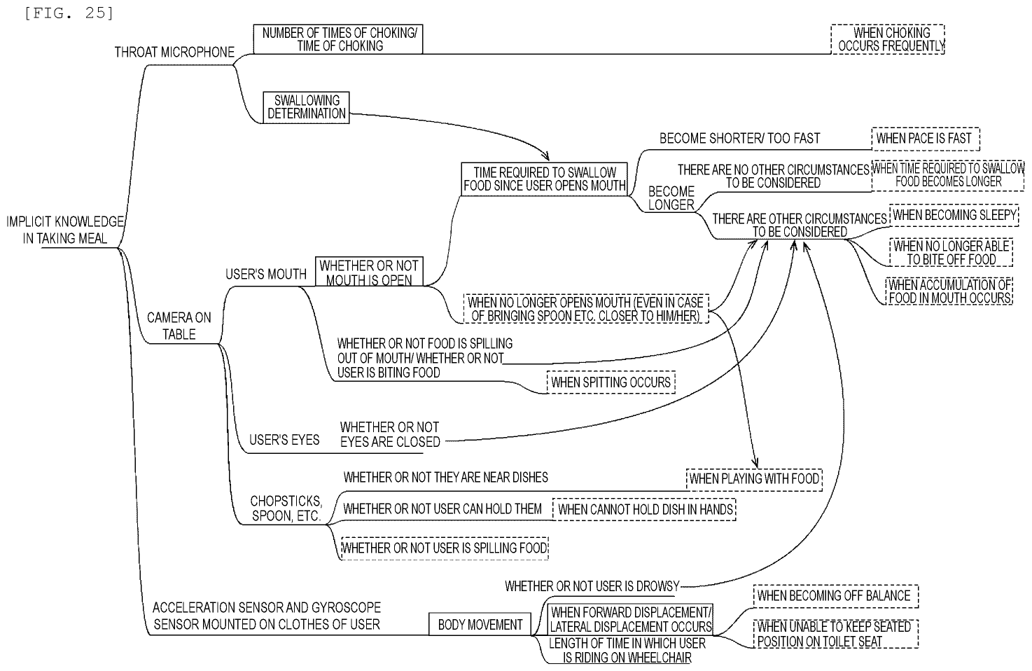

FIG. 25 is a diagram illustrating how the above devices and the situations illustrated in FIG. 23 are associated with each other. As illustrated on the left side of FIG. 25, devices used for implicit knowledge in taking a meal are the throat microphone TM, the camera of the communication device 200-5, and the acceleration sensor 120 of the wearable module 100, for example. In addition, in FIG. 25, items stated on lines extending from each device represent information that can be determined based on the device. In FIG. 25, portions surrounded by a frame of a broken line represent the situations illustrated in FIG. 23.

The throat microphone TM is configured to determine choking and swallowing of a care recipient. A device for detecting swallowing using a microphone mounted around a neck is stated in U.S. patent application Ser. No. 16/276,768, filed on Feb. 15, 2019, and entitled “SWALLOWING ACTION MEASUREMENT DEVICE AND SWALLOWING ACTION SUPPORT SYSTEM”. This patent application is incorporated herein in its entirety by reference. By using the throat microphone TM, as illustrated in FIG. 25, the processing unit 310 can detect the number of times of choking, the time of choking (such as the time when choking occurs and duration of choking), and whether swallowing is performed.

In addition, as illustrated in FIG. 24 for example, the camera of the communication device 200-5 can detect the mouth and eyes of a care recipient and chopsticks, a spoon, and the like used by the care recipient by taking images of the care recipient in the front direction. Note that, various methods for detecting the parts of the face and the objects described above based on image processing have been known, and the publicly known methods can be widely employed in this embodiment.

For example, based on images taken by the camera, the processing unit 310 can determine whether the mouth of the care recipient is open, whether food is spilling out of the mouth of the care recipient, and whether the care recipient is biting food. In addition, based on the images taken by the camera, the processing unit 310 can determine whether the eyes of the care recipient is open. Further, based on the images taken by the camera, the processing unit 310 can determine whether the chopsticks, spoon, and the like are near dishes, whether the care recipient can hold them, and whether the care recipient is spilling food.

The method of this embodiment presumes the situation of a care recipient based on information that can be identified from these devices. For example, the processing unit 310 may perform processing of identifying an action to be executed by a caregiver based on the result of detection of choking and swallowing and the result of determination on whether the mouth of the care recipient is open or closed.

For example, as illustrated in FIG. 25, based on the number of times of choking and the time of choking, it is possible to determine whether the situation of “when choking occurs frequently” applies. For example, the processing unit 310 may determine that choking occurs frequently if the number of times of choking per unit time exceeds a threshold. This makes it possible to automatically determine the situation related to choking and thus possible to present an appropriate action to a caregiver.

In addition, as illustrated in FIG. 25, the processing unit 310 may obtain the swallowing time required for a care recipient to swallow food since he/she opens his/her mouth based on the result of detection of swallowing and the result of determination on whether the mouth of the care recipient is open or closed, and perform processing of identifying an action to be executed by a caregiver based on the swallowing time thus obtained. Detection of swallowing itself is stated in U.S. patent application Ser. No. 16/276,768. However, even if it is found that the number of times of swallowing is reduced, for example, it is not easy to determine a specific situation such as because a care recipient does not even perform an action of putting food into his/her mouth or because the care recipient has put food into his/her mouth but does not swallow it.

In that respect, by determining the swallowing time required for swallowing since a care recipient opens his/her mouth, it is possible to obtain the time required for chewing and swallowing. For example, the processing unit 310 may start counting up with a timer when it is found based on images taken by the communication device 200-5 that a care recipient transitions from a state of closing his/her mouth to a state of opening his/her mouth, and stop the measurement with the timer when swallowing is detected by the throat microphone TM. The time when the timer stops represents the swallowing time. This makes it possible to precisely determine whether a care recipient is in a situation where a caregiver should execute some sort of action in taking a meal, and thus possible to use the implicit knowledge of a skilled worker appropriately.

For example, if the swallowing time is short, it is possible to determine that a care recipient is in a situation of “when pace is fast”. Meanwhile, if the swallowing time is long, the processing unit 310 may determine whether there are other circumstances to be considered based on the result of determination on other situations using the devices. Note that, the processing unit 310 may determine whether the swallowing time is long based on a change in the swallowing time during one meal (such as the amount of increase in the swallowing time with respect to that in the initial phase and the ratio of the swallowing time to that in the initial phase).

Alternatively, the processing unit 310 may obtain average swallowing time etc. of a single care recipient for every time of meals, and determine whether the swallowing time becomes longer based on a change in the average swallowing time.

For example, by using the result of determination on whether the mouth of a care recipient is open or closed based on images taken by the communication device 200-5, it is possible to determine whether the care recipient is in a situation of “no longer opens his/her mouth” even if a caregiver brings a spoon and the like closer to the care recipient. If the swallowing time becomes longer under a situation where the care recipient is not willing to open his/her mouth, it is possible to presume that the care recipient is in a situation of “accumulation of food in his/her mouth occurs”. In addition, by using the result of mouth recognition using taken images, i.e., whether food is spilling out of the mouth of the care recipient and whether the care recipient is biting food, it is possible to determine whether the care recipient is in a situation of “no longer able to bite off food”. For example, if the swallowing time is long although the number of times of chewing is as usual, it is possible to presume that the care recipient is in a situation of “no longer able to bite off food”. Meanwhile, if it is determined using taken images that the eyes of the care recipient are closed, it is possible to determine that the care recipient is in a situation of “becoming sleepy”. Note that, the above is merely an example of the situation determination, and the processing contents are not limited to this. For example, the processing unit 310 may presume that a care recipient is in a situation of “accumulation of food in his/her mouth occurs” if determining based on taken images that the care recipient is spitting food from his/her mouth. For example, in the case of a care recipient whose dementia progresses, accumulation of food in his/her mouth may occur when he/she forgets the fact that he/she is eating food and opens his/her mouth. For example, on the basis of the attributes of a care recipient such as the degree of progress of dementia, the processing unit 310 may switch the contents of the situation determination processing based on data from the devices.

In addition, as illustrated in FIG. 25, it may be determined whether a care recipient is drowsy based on the above falling down determination processing. For example, the processing unit 310 determines that a care recipient is drowsy in cases where the care recipient becomes off balance compared to the normal state, where periodic swing of his/her body is detected, and the like. In this case, the processing unit 310 determines that the care recipient is in a situation of “becoming sleepy” as in the case where the care recipient is closing his/her eyes.

On the other hand, if it is found by referring to other situation determination results that there are no circumstances why the swallowing time becomes longer, the processing unit 310 determines that a care recipient is in a situation of “the time required to swallow food becomes longer”. As an example, this corresponds to a case where a care recipient becomes full; however, a device for sensing to what extent the care recipient is full is not assumed here, and therefore it is not directly determined whether the care recipient becomes full.

In addition, as illustrated in FIG. 25, through processing of recognition of chopsticks, a spoon, and the like using taken images, it may be determined whether a care recipient is in any of situations such as “playing with food”, “cannot hold a dish in his/her hands”, and “spilling food”. Further, a situation such as “becoming off balance” may be determined based on the above falling down determination processing.

As described above, it is possible to determine the situation of a care recipient by using the output of each device appropriately. In addition, as illustrated in FIG. 23, by holding information associating the situation with the action as the implicit knowledge of a skilled worker, it is possible to present to a caregiver an appropriate action according to the situation. For example, the action may be presented to a caregiver by outputting voice to the headset 420, may be presented by displaying it on the display of the mobile terminal device 410, or may be presented using other methods. For example, since a care recipient is sitting on the wheelchair 520, it is possible to give notification by emission of light at a light emission unit provided on the wheelchair 520.

In particular, in the method of this embodiment, as described previously, by using the swallowing time required for a care recipient to swallow food since he/she opens his/her mouth as a main condition, it is possible to appropriately determine whether a basic operation in taking a meal, i.e. putting food into his/her mouth, chewing it, and swallowing it, is hampered. Further, by using other situation determination results in combination as additional conditions, it is possible to narrow down a specific reason why it takes time for swallowing, and thus possible to presume more detailed situation and present more appropriate action. As a result, it is possible to give a caregiver instructions suitable for the situation in taking a meal, and thus possible to use the implicit knowledge of a skilled worker appropriately.

In addition, in the method of this embodiment, the processing unit 310 may perform control to increase the number of times the sensor in the wearable module 100 is activated if an action of stopping the meal is presented. For example, the wearable module 100 may include a temperature sensor in addition to the acceleration sensor 120. In a case where the wearable module 100 is secured to the skin of a care recipient, for example, the temperature sensor can measure the temperature of a body surface, so that the body temperature of a care recipient can be presumed based on the measurement value.

By doing so, in a case where there is a possibility of aspiration pneumonitis, for example, it is possible to monitor vital information of a care recipient appropriately. A period during which the temperature sensor becomes active may be about several hours, may be about several days, or may be another period since the event of stopping the meal is detected. Further, in a case where the wearable module 100 includes sensors capable of detecting heartbeat, respiration, SpO2, and the like, these sensors may be activated with presentation of the action of stopping the meal as a trigger.

### 3.2 Position Adjustment

On the bed 510 and the wheelchair 520, the position of a care recipient needs to be adjusted. For example, the position adjustment on the bed 510 is useful for measures against bed sore. Meanwhile, the position adjustment on the wheelchair 520 is useful for measures against slipping off and measures against bed sore. Accordingly, if it is determined that a care recipient is on the bed 510 based on the result of communication between the wearable module 100 and the communication device 200, processing of supporting assistance in adjustment of the bed position may be executed. Likewise, if it is determined that a care recipient is on the wheelchair 520, processing of supporting assistance in adjustment of the wheelchair position may be executed. Hereinbelow, a specific example will be described.

### 3.2.1 Bed Position Adjustment

FIG. 26 is a diagram illustrating devices arranged in the vicinity of the bed 510. As illustrated in FIG. 26, the devices mentioned here include: the communication device 200-1 that is fixed on the foot board side of the bed 510; a second terminal device CP2 that is fixed on the side rail of the bed 510; and a display DP that is fixed on the opposite side of the second terminal device CP2. Note that, the second terminal device CP2 may be the communication device 200 according to this embodiment or may be a device that does not function as the communication device 200. In addition, while the communication device 200 corresponding to the bed 510 is provided at another position such as the wall surface of the living room, another terminal device that does not function as the communication device 200 may be used instead of the communication device 200-1. In addition, the display DP is not limited to one that is fixed on the bed 510, and may be disposed at another position such that a caregiver who adjusts the bed position can naturally view it. For example, the display DP may be fixed on the wall surface or may be fixed, for example, on a support that stands on the floor surface on its own. In addition, one of the communication device 200-1 and the second terminal device CP2 may be omitted. For example, the following description will be given of an example where the bed position is adjusted using the communication device 200-1. For example, the second terminal device CP2 is used for changing a diaper which will be described later. In addition, the communication device 200-1 may be used for changing a diaper.

The communication device 200-1 and the second terminal device CP2 are devices such as a smartphone having a camera. The communication device 200-1 is configured to transmit a taken image to the server system 300 directly. The second terminal device CP2 is configured to transmit, directly or via the communication device 200-1, an image taken by the camera to the server system 300. The display DP is configured to receive, directly or via another device such as the communication device 200-1, the image transmitted by the server system 300 and display the image thus received. Note that, the communication device 200-1 and the second terminal device CP2 may have a depth sensor instead of or in addition to the camera. In other words, these devices may output a depth image.

For example, in the bed position adjustment, processing of registering labeled training data and the position adjustment processing using the labeled training data may be executed. The labeled training data is information registered by a skilled caregiver, for example. A caregiver who is an unskilled worker selects labeled training data when adjusting the bed position, and adjusts the bed position so that an actual state of a care recipient becomes closer to that of the labeled training data. For example, the communication device 200-1 acquires an image in which a state where a care recipient whose bed position is to be adjusted is lying on the bed (including a state of a cushion and the like) is taken, and the display DP displays an image representing a result of comparison between the taken image and the labeled training data. This enables the caregiver to perform the position adjustment in the same way as a skilled worker irrespective of the degree of proficiency of the caregiver.

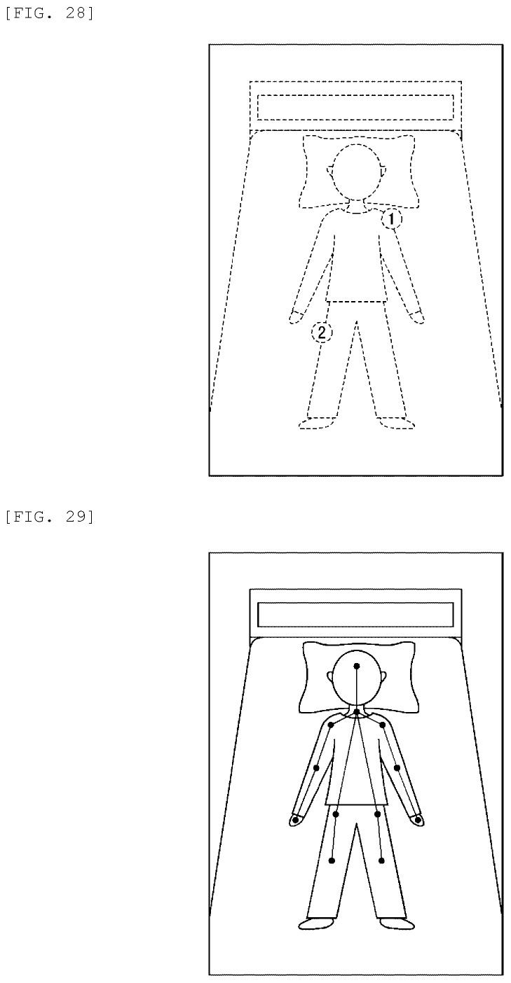

FIG. 27 illustrates an example of a registration screen of labeled training data. FIG. 27 is an image including an image taken by the communication device 200-1, for example, and is a screen displayed on the display of the mobile terminal device 410 of a skilled worker, for example. Note that, an image for labeled training data may be taken using the mobile terminal device 410. In addition, labeled training data may be registered using a device other than the mobile terminal device 410.

A skilled worker lays a care recipient on the bed 510, places him/her at a position preferable for measures against bed sore etc., and takes an image of the target care recipient using the communication device 200-1. The display of the mobile terminal device 410 may display images taken by the communication device 200-1 in real time as a moving image, or may display a still image taken by the communication device 200-1. The skilled worker selects a registration button after confirming that the care recipient is placed at an appropriate bed position. The mobile terminal device 410 transmits a still image, which is displayed when the registration button is operated, to the server system 300 as labeled training data. This makes it possible to register a position, which the skilled worker thinks is preferable, as labeled training data.

In this event, the mobile terminal device 410 may accept an input operation of additional information by the skilled worker. For example, by using a user interface unit such as a touch panel of the mobile terminal device 410, the skilled worker may perform an operation of selecting a point that is considered to be particularly important. For example, the user who is the skilled worker performs an operation of acquiring an image taken in a state where the care recipient is placed at an appropriate bed position and an operation of adding additional information, and then selects the registration button illustrated in FIG. 27.

In the example of FIG. 27, the vicinity of the left shoulder and the vicinity of the right knee of the care recipient are selected. Meanwhile, the mobile terminal device 410 may be capable of not only accepting designation of the position but also accepting inputs of a specific text and the like. For example, the skilled worker not only designates a portion such as the left shoulder but also inputs a text of points, which are important to place the care recipient at the appropriate bed position, such as the angle of this portion to another portion and the positional relationship between this portion and a pillow or cushion. The same goes for the vicinity of the right knee. In addition, when inputs designating multiple points are made, the mobile terminal device 410 may accept inputs of the degree of priority of each position. For example, in a case where a smaller value indicates a higher priority, when accepting user inputs indicating that the priority of the vicinity of the left shoulder is relatively high, the mobile terminal device 410 sets the priority of the vicinity of the left shoulder to 1 and sets the priority of the vicinity of the right knee to 2.

Meanwhile, when a caregiver adjusts the bed position in practice, the caregiver first activates the communication device 200-1 and starts taking images. For example, the caregiver activates the communication device 200-1 by voice, and the display DP displays a moving image taken by the communication device 200-1. In addition, the processing unit 310 of the server system 300 may accept labeled training data selection processing by the caregiver. For example, the processing unit 310 may display a list of labeled training data on the display of the mobile terminal device 410. The processing unit 310 performs control to determine labeled training data based on a selection operation at the mobile terminal device 410 and display this labeled training data on the display DP.

Alternatively, the processing unit 310 may perform processing of automatically selecting labeled training data based on determination on similarity between the attributes of a care recipient whose bed position is to be adjusted and the attributes of a care recipient whose images are taken in labeled training data. The attributes mentioned here include information on the age, sex, height, weight, past medical history, medication history, and the like of the care recipient.

Alternatively, the processing unit 310 may perform processing of automatically selecting labeled training data based on processing of comparison between the attributes of a care recipient whose bed position is to be adjusted and additional information included in labeled training data. For example, assume that a text indicating that “For a care recipient who has a tendency of XX, it is preferable to make adjustment such that the left shoulder may be YY” is included as additional information of labeled training data. In this case, if the care recipient whose bed position is to be adjusted corresponds to XX, selection of this labeled training data is easy. For example, a caregiver who makes the bed position adjustment may transmit information identifying the care recipient to the server system 300 via the mobile terminal device 410 and the like, and the processing unit 310 may identify the attributes of the care recipient based on this information.

Meanwhile, the processing unit 310 may classify care recipients into several classes using the result of determination in the falling down determination processing and the assessment devices such as Waltwin and SR AIR described above. Then, the processing unit 310 may perform processing of automatically selecting labeled training data based on processing of comparison between the class of a care recipient whose bed position is to be adjusted and the class of a care recipient whose images are taken in labeled training data.

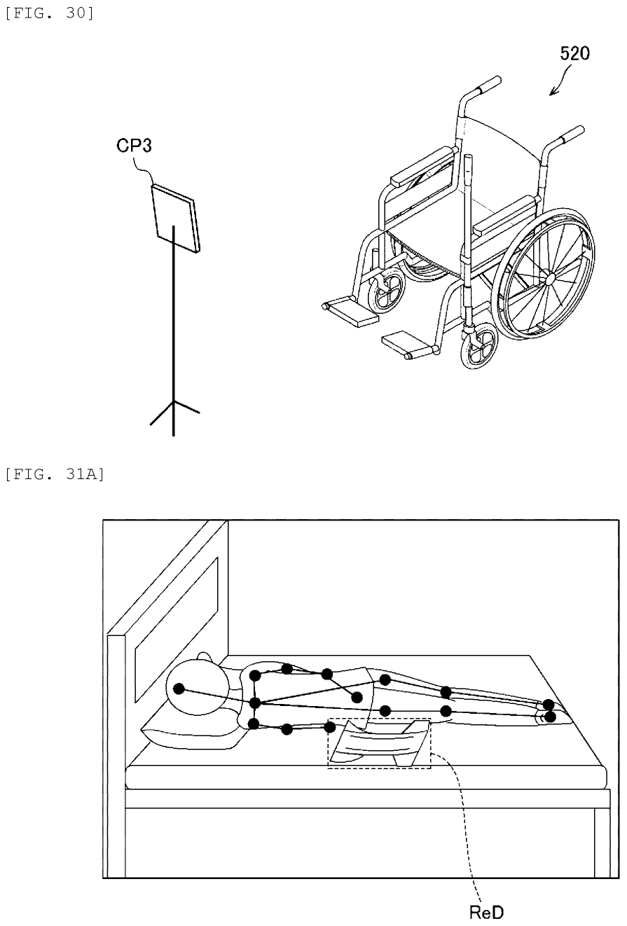

For example, the processing unit 310 may perform processing of displaying images, taken by the communication device 200-1 in real time, while superimposing labeled training data having been subjected to transparent processing on the images. FIG. 28 illustrates an example of an image displayed while the labeled training data illustrated in FIG. 27 is superimposed thereon. By making adjustment such that an actual care recipient and the care recipient of the labeled training data overlap each other in this manner, even a caregiver with a low degree of proficiency can adjust the bed position easily.

In addition, as illustrated in FIG. 28, the additional information of the labeled training data may be displayed so as to be recognizable. For example, in FIG. 28, objects being circled numbers are respectively displayed at positions of the vicinity of the left shoulder and the vicinity of the right knee that are designated by the skilled worker. The caregiver who makes the bed position adjustment can understand important points by viewing the objects. In addition, when an operation of selecting the object is performed, the processing unit 310 may display a text added by the skilled worker on the display DP. Further, when it is detected that a caregiver has spoken “let me know the points” with the microphone of the headset 420, the processing unit 310 may output a text by voice from the headset 420.

For example, based on the degree of similarity between an image taken during the position adjustment and labeled training data, the processing unit 310 determines whether it is OK or NG, and displays the determination result on the display DP. Alternatively, the processing unit 310 may output the determination result by voice from the headset 420. In addition, the processing unit 310 may perform processing of displaying a specific point why it is determined as NG. For example, the processing unit 310 may perform processing of comparing an image taken by the communication device 200-1 with labeled training data and highlighting a point where the difference is determined to be large.

In this way, by providing the display DP at a position different from that of the communication device 200-1 that takes an image, e.g. at a position on the side frame side, a caregiver can adjust the position of a care recipient while visually checking the display DP in a natural posture. Since a caregiver does not need to view an image taken by the communication device 200-1 using the display of the communication device 200-1, the level of convenience can be increased.

In this event, as illustrated in FIGS. 27 and 28, it is possible to register a point, which a skilled worker thinks is important, as additional information, and present the additional information to a caregiver. If merely seeing an image of labeled training data, a caregiver with a low degree of proficiency may be able to imitate its position but cannot understand a particularly important point, and therefore cannot prioritize points in the position adjustment. In that respect, according to the method of this embodiment, since a skilled worker's intention is presented clearly, even a caregiver with a low degree of proficiency can use implicit knowledge appropriately.

In addition, when an image is displayed while labeled training data being a picture is superimposed thereon as illustrated in FIGS. 27 and 28, information on objects such as a cushion that exist in the background is stored in the labeled training data. This is advantageous in that the positional relationship between a care recipient and the cushion can also be adjusted appropriately.

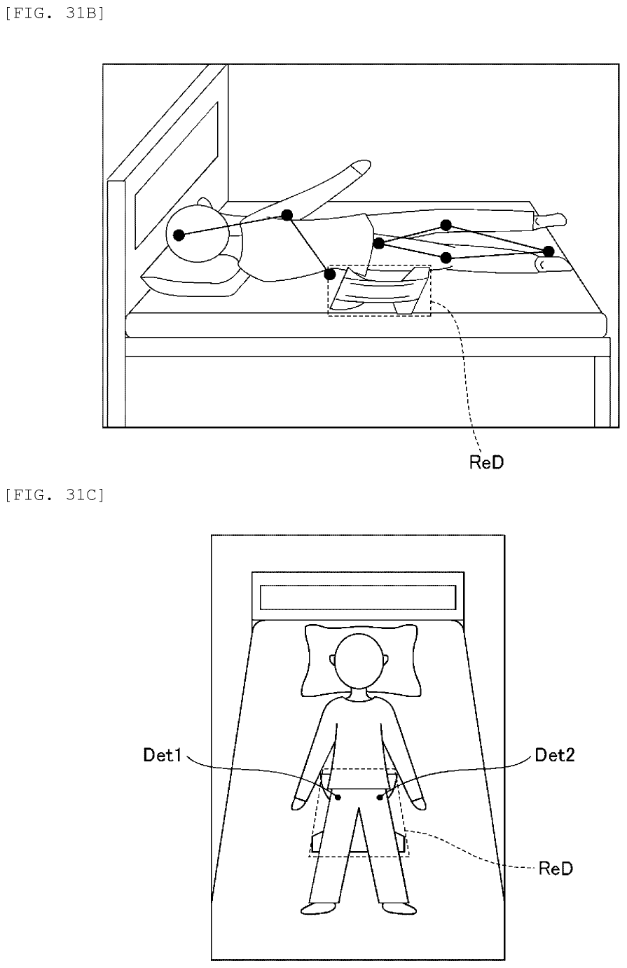

FIG. 29 is a diagram illustrating another method of the bed position adjustment, and is a diagram illustrating a skeleton tracking result. Note that, for the method of skeleton tracking based on an image, various methods such as OpenPose disclosed in Realtime Multi-Person 2D Pose Estimation using Part Affinity Fields (https://arxiv.org/pdf/1611.08050.pdf) by Zhe Cao and others have been known, and these methods can be widely employed in this embodiment.

For example, when registering labeled training data, as in the example described above, a skilled worker lays a care recipient on the bed 510, places him/her at a position preferable for measures against bed sore etc., and takes an image of the target care recipient using the communication device 200-1. The processing unit 310 performs skeleton tracking on the taken image, and displays a predetermined number of positions which are the result of the skeleton tracking on the taken image. The number of points tracked is 17, for example, but is not limited to this.

The processing unit 310 may include the entire skeleton tracking result in labeled training data.

Alternatively, the processing unit 310 may accept an operation of selecting a part of the points detected by the skeleton tracking. For example, a skilled worker designates three points which he/she thinks are important in the bed position adjustment. As an example, the skilled worker may designate three points including the shoulder, waist, and knee. However, a combination of portions to be designated is not limited to this, and the number of portions to be designated is not limited to three.

In the bed position adjustment using labeled training data, as in the example described previously, the camera of the communication device 200-1 acquires an image in which a care recipient is taken. The server system 300 performs skeleton tracking on the image thus taken, and performs processing of displaying the processing result on the display DP. For example, as in the image in FIG. 29, the processing result indicates an image which is displayed while the skeleton tracking result of the taken image that is registered as labeled training data, an image that is being taken by the camera of the communication device 200-1, and the skeleton tracking result of the image that is being taken are superimposed one on top of the other. In this case, the taken image, registered as labeled training data, itself is not displayed. Note that, in this event, all the points detected by the skeleton tracking may be displayed, or alternatively only a part of the points designated by a skilled worker may be displayed.

The bed position adjustment using the skeleton tracking result is superior to the overlapping between the taken images (the image in the labeled training data and the image being taken) described previously in that it can be employed even when equipment such as a cushion used by a care recipient is different between the images and is highly versatile.

The processing unit 310 performs processing of comparing the three points of the shoulder, waist, and knee in the labeled training data and the three points of the shoulder, waist, and knee in the taken image. For example, the processing unit 310 may determine whether the three points of the shoulder, waist, and knee are at their desired angles, or may determine whether these three points are within a certain linear range. The processing unit 310 determines whether it is OK or NG, for example, and displays the determination result on the display DP. Alternatively, the processing unit 310 may output the determination result by voice from the headset 420. In addition, the processing unit 310 may perform processing of displaying a specific point why it is determined as NG.

Note that, the foregoing description has been given of the bed position adjustment in the case of laying a care recipient on the mattress that is parallel (including substantially parallel) to the floor surface. However, the bed position adjustment is not limited to this, and the bed position adjustment may be performed according to a situation (scene) of the care recipient.

Meanwhile, the bed position adjustment may be performed by controlling the bed 510 or the like. For example, if choking etc. occurs while a care recipient is taking a meal due to his/her posture, it is possible to let the care recipient take a meal smoothly by performing control to change the angle of the sections of the bed 510. The control to change the angle of the sections includes control such as lifting the back board and lifting and tilting the waist board.

For example, a skilled worker may register labeled training data in association with information identifying a target situation. In the above example, the target situation corresponds to the situation of “taking a meal” and “choking occurs frequently”, and labeled training data obtained by associating the image, in which the care recipient having been subjected to adjustment of the sections is taken, with a tag etc., indicating that choking is caused by the “posture”, is acquired. A caregiver who provides assistance in practice performs the bed position adjustment of changing the angle of the sections based on the labeled training data. Alternatively, the control to change the angle of the sections may be executed automatically, and the caregiver may provide assistance in fine adjustment of the bed position based on the labeled training data. In other words, control over the bed 510 which is the peripheral device 700 may be performed in addition to or instead of the notification to the caregiver terminal 400.

In addition, the processing unit 310 may determine the situation based on the device and select labeled training data automatically based on the determination result. For the situation determination, the same method as that in the processing described above using FIGS. 23 and 25 can be used, for example. For example, the processing unit 310 makes the above labeled training data easy to select when it is determined through the throat microphone TM that a care recipient is in a situation where choking occurs frequently and when it is determined through the falling down determination processing by the acceleration sensor 120 that this is caused by his/her posture.

Note that, when choking is detected while asleep, the bed position may be adjusted by control over a pillow instead of control over the bed 510. For example, the following URL discloses Motion Pillow that has an airbag embedded therein and that prompts the user to turn over when detecting snoring by expanding the embedded airbag. For example, when detecting that the user is in a situation of “sleeping” and “choking occurs”, the processing unit 310 may prompt the user to move to the lateral position by controlling the airbag of the pillow. In other words, the peripheral device 700 that is a target for intervention control may include a pillow.
http://www.motionpillow.com/

### 3.2.2 Position on Wheelchair

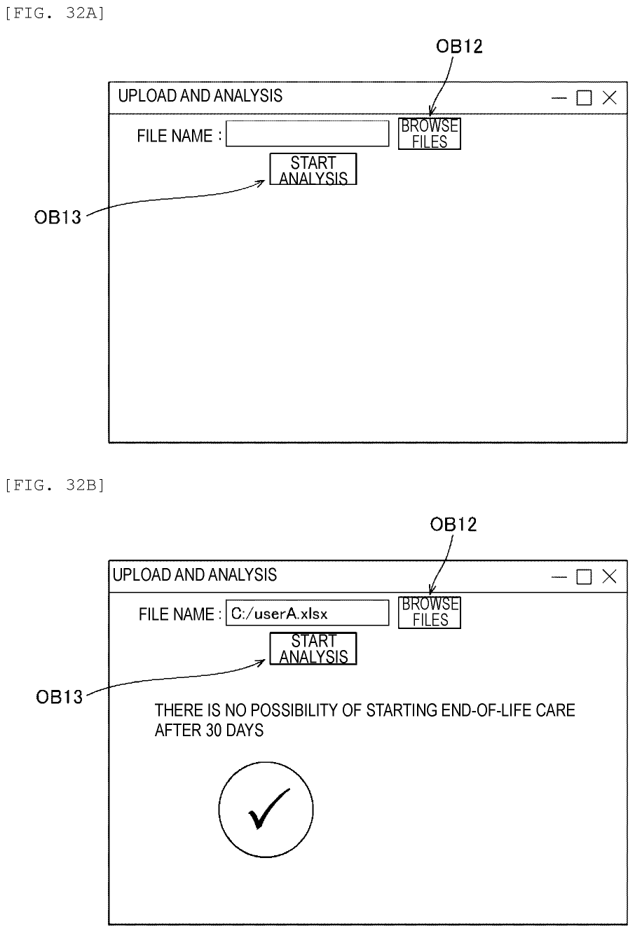

FIG. 30 is a diagram illustrating a system configuration at the time of adjustment of the wheelchair position. As illustrated in FIG. 30, for the wheelchair position adjustment, a third terminal device CP3 that includes a camera and is fixed at a height such that the camera can take an image of at least the upper body of a care recipient who is sitting on the wheelchair 520 may be used. Note that, the third terminal device CP3 may be capable of taking an image of a larger range of the care recipient, and may take an image of the care recipient from the top to knees or may take an image of the whole body of the care recipient, for example. For example, the third terminal device CP3 is disposed at a predetermined position of a nursing care facility, and a caregiver performs the wheelchair position adjustment after moving the care recipient to the wheelchair 520 and moving him/her to the front of the third terminal device CP3.

The third terminal device CP3 includes a display that displays a result of comparison between an image taken by the camera and labeled training data. A method of registering labeled training data is the same as that in the case of the bed position, and the labeled training data may be data in which additional information is added to a taken image as illustrated in FIG. 27 or may be data in which a skeleton tracking result is added to a taken image as illustrated in FIG. 29. On the display of the third terminal device CP3, the processing unit 310 may display an image while superimposing labeled training data having been subjected to transparent processing on the image, or may display the difference obtained in the skeleton tracking result.

Note that, in the case of using the system illustrated in FIG. 30, since the camera of the third terminal device CP3 can take an image of a care recipient from the front, the camera can take an image of the care recipient's face more clearly than in the case of using the device fixed on the bed 510 as the communication device 200-1 in FIG. 26. Accordingly, in the case of selecting labeled training data automatically according to a care recipient, the processing unit 310 may automatically identify a care recipient whose position is to be adjusted based on a result of face recognition processing.

Note that, instead of the third terminal device CP3, the communication device 200-5 disposed on the table for taking a meal illustrated in FIG. 24, for example, may be used. In this case, since it is not easy for the camera of the communication device 200-5 to take an image of the lower body of a care recipient, the processing unit 310 performs determination processing based on the upper body of the care recipient. Note that, in the case of using the communication device 200-5 illustrated in FIG. 24, the processing unit 310 may detect forward displacement and lateral displacement based on the taken image. For example, the processing unit 310 determines that forward displacement occurs if the head position and the shoulder position are lowered and determines that lateral displacement occurs if the head position and the shoulder position are displaced laterally as compared to those observed when the care recipient starts taking a meal.

The same goes for the case of the wheelchair position with regard to the point that the position adjustment may include control over devices and the like. For example, if choking etc. occurs while a care recipient is taking a meal due to his/her posture, the processing unit 310 may automatically perform control such as lifting the backrest up, fastening sealings, and pulling back the seat surface, or may perform processing of prompting a caregiver to perform such control by presenting it to the caregiver. For example, in the case of detecting the posture using the pressure sensor illustrated in FIG. 18, the processing unit 310 may keep performing the above control until it is determined based on the pressure sensor that the position of the center of gravity returns back to the normal state.

Meanwhile, in the case of a care recipient who uses the wheelchair 520, the care recipient can at least keep a seated position, and therefore might be able to adjust his/her posture by himself/herself. In this case, a device with a relatively large display size may be used as the third terminal device CP3. In this case, since an image in which a care recipient is taken is displayed on the third terminal device CP3 located at the front, the care recipient can use the third terminal device CP3 like a full-length mirror. For example, as described previously, by displaying a point to be corrected on the third terminal device CP3, it is possible to prompt a care recipient to correct the posture of the care recipient by himself/herself.

### 3.3 Changing of Diaper

It has been found out that a skilled worker places great importance on the following points as implicit knowledge in changing a diaper.
A. Whether a care recipient is in a lateral position
B. Whether the position of a diaper is appropriate
C. Whether a pad sticks out of a diaper
D. Whether a diaper is mounted properly

Accordingly, in this embodiment, it is determined whether the above points A to D are satisfied to present the determination result. This enables a caregiver to change a diaper properly irrespective of the degree of proficiency of the caregiver.

A system used in changing a diaper is the same as that in FIG. 26, for example. For example, the second terminal device CP2 transmits a moving image, in which a care recipient is taken using the camera, to the server system 300 directly or via the communication device 200 disposed on the bed 510. The processing unit 310 of the server system 300 performs skeleton tracking processing on each image constituting the moving image, and displays on the display DP an image obtained by superimposing the skeleton tracking result on the original image. By doing so, a caregiver can check the display DP in a natural posture while changing a diaper of a care recipient.

Note that, in consideration of the case of changing a diaper in the nighttime, the second terminal device CP2 may include a lighting unit. In addition, in consideration of a care recipient's privacy, a depth sensor or the like may be used instead of the camera. The depth sensor may be a sensor using the Time of Flight (ToF) method, may be a sensor using structured illumination, or may be a sensor using other methods.

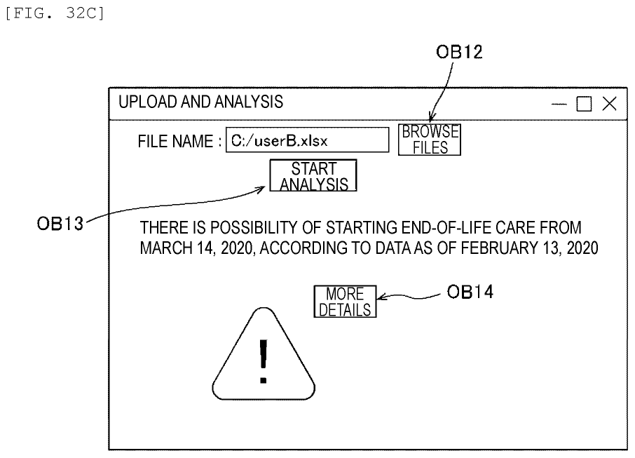

FIGS. 31A and 31B illustrate an example of images displayed on the display DP in the case of changing a diaper. As described previously, each image includes a care recipient and the care recipient's skeleton tracking result.

In a state of FIG. 31A, a care recipient is laid stably in a lateral position, and the camera of the second terminal device CP2 takes an image of the care recipient from his/her back straight from the camera. For example, in FIG. 31A, the difference between the length in the front-rear direction of the body of the care recipient and the length in the optical axis direction of the camera is small. As a result, as illustrated in FIG. 31A, many points are detected as points to be detected by the skeleton tracking.

On the other hand, in FIG. 31B, the posture is not stable as compared with that in FIG. 31A, and a care recipient is in a state of being likely to fall on his/her back. The camera of the second terminal device CP2 is in a state of taking an image of the care recipient from obliquely behind, and therefore the number of points detected by the skeleton tracking decreases. For example, a point corresponding to the waist is hidden by a diaper and the like and not detected.

Accordingly, the processing unit 310 may determine based on the skeleton tracking result whether the care recipient is in a lateral position as stated in A above. For example, the processing unit 310 may determine that the care recipient is in a lateral position if a point corresponding to a specific portion such as the waist is detected by the skeleton tracking. However, a specific method for the lateral position determination is not limited to this, and whether a point other than the waist is detected, the relationship between multiple points, and the like may be used.

In addition, the processing unit 310 continuously detects a diaper region in an image through object tracking processing based on the moving image from the second terminal device CP2. Since the object tracking is publicly known, its detailed description will be omitted. For example, in FIGS. 31A and 31B, a diaper region ReD is detected.

The processing unit 310 may determine whether the position of a diaper is appropriate as stated in B above based on the relationship between the skeleton tracking result and the diaper region ReD detect by the object tracking, for example. For example, while taking into consideration a position where a diaper is to be mounted, the processor determines whether the waist position detected by the skeleton tracking and the diaper region ReD have a predetermined positional relationship. For example, the processing unit 310 may determine that the position of the diaper is appropriate if a straight line including two points corresponding to the pelvis passes through the diaper region ReD. Alternatively, machine learning may be performed in such a way that the skeleton tracking result from labeled training data of a skilled worker and the result of detection of the diaper region ReD are extracted as feature data and the feature data is set as input data. The learned model is a model that outputs accuracy on whether the position of a diaper is appropriate upon receiving the skeleton tracking result and the result of detection of the diaper region ReD, for example.

Meanwhile, the processing unit 310 may determine whether a pad sticks out of a diaper as stated in C above based on the length of the diaper region ReD in a horizontal direction. Since the pad is normally supposed to be fitted into the diaper, the length of the diaper region ReD in an image corresponds to the length of the diaper itself. Note that, the assumed size of the diaper region ReD can be presumed based on the type and size of the diaper, the optical characteristics of the camera of the second terminal device CP2, and the like. On the other hand, when the pad sticks out, the length of the diaper region ReD in the image is longer by the amount that it sticks out. Accordingly, if the length of the diaper region ReD detected in the image is larger than the assumed length by a predetermined threshold or more, the processing unit 310 determines that the pad sticks out of the diaper and is thus inappropriate.

Meanwhile, the processing unit 310 may determine whether a diaper is mounted properly as stated in D above by detecting a tape for fixing the diaper in a state where the diaper is mounted. Normally, a member with a color different from the diaper main body is used for the tape. As an example, the diaper main body is white while the tape is blue. In addition, where and how the tape should be fixed in order to mount the diaper properly are known from the structure of the diaper. Accordingly, the processing unit 310 can detect a tape region in an image based on its color, and determine whether the diaper is mounted properly based on the relationship between the tape region and the diaper region ReD or based on the positional relationship between the tape region and the waist etc. detected by the skeleton tracking. Note that, in a case where multiple diapers of different manufacturers and types are used, the processing unit 310 may acquire information identifying a diaper and determine whether a diaper is mounted properly based on the type of the diaper etc. thus identified.

With the above processes, it is possible to use implicit knowledge in changing a diaper appropriately and cause a caregiver to change a diaper properly. For example, the processing unit 310 determines whether it is OK or NG for each of A to D above, and displays the determination result on the display DP. Further, if determining that it is NG, the processing unit 310 may highlight a portion having a large difference from ground truth data.

Note that, the processes described above are an example of automating the determinations of A to D above using the device, and other methods may be used. For example, a pressure sensor may be used instead of the skeleton tracking for determining a lateral position. For example, a pressure sensor may be disposed at a position closer to the side frame than the center of the bed or mattress (positions displaced rightward and leftward relative to the center). A caregiver can move a care recipient, who is lying on the bed with face up at a position near the center, to a lateral position by turning the care recipient leftward or rightward by 90 degrees. To put it differently, when a lateral position is implemented, a load applied on the pressure sensor increases since the body of the care recipient moves to the side frame side due to turning. The processing unit 310 may determine that the care recipient has moved to a lateral position if an output value of the pressure sensor is equal to or larger than a predetermined value.

Meanwhile, in the case of changing a diaper while prompting a care recipient to get up, the care recipient is assumed to grip the side rail. Accordingly, with a pressure sensor disposed on the side rail, the processing unit 310 may determine that the care recipient has moved to a lateral position if an output value of the pressure sensor is equal to or larger than a predetermined value.

Note that, in the case of determining a lateral position as stated in A above using the pressure sensor or the above depth sensor, if it is determined that a care recipient is in a lateral position, taking an image by the camera of the second terminal device CP2 and determinations of B to D described above may be started. For example, in the case of changing a diaper in the nighttime, the processing unit 310 may turn on the light of the second terminal device CP2 if determining that a care recipient becomes a lateral position. Alternatively, the processing unit 310 may turn on the light of the living room of the care recipient if determining that the care recipient becomes a lateral position. This makes it possible to control the light appropriately at the timing when processing using an image taken by the camera is needed.

Meanwhile, the processing unit 310 may execute processing on changing a diaper using the communication device 200-1 illustrated in FIG. 26. FIG. 31C illustrates an example of an image displayed on the display DP in the case of changing a diaper based on an image taken by the communication device 200-1. The output of the communication device 200-1 is an image in which a care recipient in a supine position is taken from the foot board side. As illustrated in FIG. 31C, the displayed image includes the care recipient, the skeleton tracking result of the care recipient, and the diaper region ReD. Note that, although waist detection results Det1 and Det2 are illustrated as the skeleton tracking result in FIG. 31C, detection results of other portions may be displayed as described above using FIG. 29.

For example, the processing unit 310 may determine whether a care recipient has a posture suitable for changing a diaper based on, instead of the determination of A above, processing of comparison between labeled training data representing a posture suitable for changing a diaper in a supine position and an image actually taken. For example, as in the case of the bed position adjustment, the processing unit 310 may display a taken image on the display DP while superimposing labeled training data on the image, or may compare the image with the skeleton tracking result.

Meanwhile, the processing unit 310 may determine whether it is OK or NG from the perspective of B to D above. For example, with regard to B above, the processing unit 310 may specify a trapezoidal region for the waist positions (Det1 and Det2) detected by the skeleton tracking, and determine whether a diaper is set to be fitted into the trapezoidal region. For example, the processing unit 310 may determine that it is OK if the center of the diaper is located on a perpendicular line with respect to a line segment connecting two points of the waist or located within a range in which the distance from the perpendicular line is equal to or smaller than a predetermined value and if the trapezoidal region and the diaper region ReD have a predetermined positional relationship (e.g. the trapezoidal region is included in the diaper region ReD). The trapezoidal region mentioned here is a region that is set based on the diaper region ReD in labeled training data. For example, the trapezoidal region is a region where a perpendicular bisector with respect to each of an upper base and a lower base coincides with (including substantially coincides with) a perpendicular bisector with respect to a line segment connecting the waist detection results Det1 and Det2, and is a region having a predetermined height. For example, the trapezoidal region is a region which includes the waist detection results Det1 and Det2 and in which the distance from Det1 to the upper base is equal to H1 and the distance from Det1 to the lower base is equal to H2, and H1 and H2 may be stored in the storage unit 320 or the like as parameters. However, the relationship between the waist detection results Det1 and Det2 and the trapezoidal region is not limited to this, and can be modified in various ways. In addition, the position and size of the trapezoidal region may be fixed values, or may be changed dynamically according to the positions of the waist detection results Det1 and Det2.

Since the determinations of C and D described above are made in the same manner as in the case of using the second terminal device CP2, their detailed description will be omitted.

### 3.4 Ability Presumption and Use of Presumption Result

In this embodiment, the ability of a care recipient may be presumed using the processing having been described above. The ability mentioned here includes a seating ability, a walking ability, and a swallowing ability. Hereinbelow, each of these abilities will be described.

### 3.4.1 Seating Ability

As illustrated in the falling down determination processing in the case of the wheelchair 520 and the processing on taking a meal described above using FIG. 25 etc., the method of this embodiment enables detection of forward displacement and lateral displacement in the wheelchair 520. These displacements may be detected by the wearable module 100, may be detected by the pressure sensor illustrated in FIG. 18A, or may be detected using the devices such as Waltwin and SR AIR. In addition, the forward displacement and lateral displacement may be detected when a care recipient is taking a meal on the bed 510. Likewise, in the case of the bed 510, the wearable module 100 may be used, or the pressure sensor disposed on the bed 510 may be used, or the devices such as Waltwin and SR AIR may be used.

For example, the processing unit 310 measures the time that elapses since a care recipient starts taking a meal on the wheelchair 520 until he/she becomes off balance. The level of the seating ability is evaluated by the length of this time. In addition, when the care recipient becomes off balance, the processing unit 310 may evaluate whether the displacement is forward displacement or lateral displacement (rightward displacement/leftward displacement) and the degree of this displacement. Further, the processing unit 310 may classify care recipients into multiple classes based on whether a care recipient has a seating ability, the level of the seating ability, and the degree of lateral displacement/forward displacement. In the case of using Waltwin and SR AIR, a more detailed classification may be carried out using a time series change in pressure distribution.

Note that, evaluation of the seating ability such as the JSSC-version displacement degree measurement is the method heretofore known. However, in this embodiment, since it is possible to use results of processing executed in daily assistance to a care recipient, such as taking a meal and the falling down determination processing, the seating ability can be presumed more easily than in the existing method.

In this manner, the processing unit 310 of this embodiment may presume the seating ability, which represents the ability of a care recipient to keep a seated position, based on sensor information corresponding to the bed 510 or sensor information corresponding to the wheelchair 520. Note that, the sensor information mentioned here corresponds to the output of the acceleration sensor 120 of the wearable module 100; however, as described previously, the output of another sensor may be used for presuming the seating ability. Then, based on the seating ability thus presumed, the processing unit 310 may execute determination processing on assistance at other locations including at least the toilet 600.

For example, the processing unit 310 uses the seating ability presumption result for situations such as determination on whether assistance should be provided when a care recipient is in the toilet, change of parameters (such as a threshold for forward fall) in the falling down determination processing in the toilet, and change of parameters in the falling down determination processing during walking. For example, if the seating ability is high, determinations such as one that no assistance is required and one that the risk of falling down is low even if a care recipient becomes off balance to some extent are likely to be made. In addition, changes such as one that the risk of falling down in a certain direction is likely to be evaluated as high while the risk of falling down in another direction is likely to be evaluated as low may be made according to the tendency of forward displacement and lateral displacement.

In this way, the ability presumption result based on sensor information at a certain location may affect processing at another location. To put it differently, in a case where information that is applicable irrespective of the location such as the ability of a care recipient is requested, by sharing this information with other locations, it is possible to enhance processing precision at each location.

### 3.4.2 Walking Ability

As illustrated in the falling down determination processing during walking, the method of this embodiment enables detection of the risk of falling down during walking. For example, as described previously, the processing unit 310 may determine the risk of falling down based on whether a periodic swinging rhythm in the left-right direction becomes off balance.

The processing unit 310 evaluates the walking ability based on the length of time that elapses since a care recipient starts walking until the risk of falling down increases. In addition, the processing unit 310 may evaluate the way of falling down such as a forward fall or a rearward fall and the degree of falling down. The walking ability may be evaluated based on the evaluation on the seating ability as described above.

However, server load may increase if all cases of falling down that may occur during walking are determined in real time. To deal with this, in this embodiment, assessment of walking may be performed using Waltwin described above. For example, based on the output of Waltwin, the processing unit 310 determines the position of the center of gravity (whether the center of gravity is shifted forward or rearward), the time during which the foot is on the ground, the order in which the pressure is released, and in what speed the pressure is applied in chronological order, for example. Then, based on these pieces of information, the processing unit 310 may narrow down a pattern by which the rhythm becomes off balance and execute the falling down determination processing with this pattern set as a detection target.

For example, a care recipient whose center of gravity tends to be shifted rearward is likely to fall rearward. In the case of a rearward fall, a signal value on the Y axis often increases gradually. For example, in the case of a rearward fall, a lower peak value and an upper peak value of a periodic signal increase with time. Accordingly, if it is already known by assessment that a care recipient tends to fall rearward, the processing unit 310 makes a determination in the falling down determination processing only on whether an acceleration value increases gradually, so that processing load can be decreased. As described above, since the walking ability presumption processing can be performed using the result of the falling down determination processing, it is possible to decrease processing load caused by the walking ability presumption processing.

As another example, in a case where the time during which the foot of a care recipient is on the ground is long, for example, an event that the rhythm becomes off balance can be detected as a change in the time during which the foot is on the ground. Accordingly, the processing unit 310 may perform the falling down determination processing based on a change in the time during which the foot is on the ground. Alternatively, in the case of a care recipient who tends to apply a pressure slowly, an event that the rhythm becomes off balance is shown as an inclination in the pressure values or a change in the period. Accordingly, the processing unit 310 may obtain an inclination in the acceleration values and the period, and may perform the falling down determination processing based on their change. Also in these methods, processing only on patterns that a care recipient is likely to have can be performed, processing load can be decreased.

In this manner, the processing unit 310 of this embodiment may presume the walking ability, which represents the ability of a care recipient to walk stably, based on sensor information corresponding to walking. Then, based on the walking ability thus presumed, the processing unit 310 may execute determination processing on assistance at other locations including at least the toilet 600.

For example, the processing unit 310 uses the walking ability presumption result for situations such as determination on whether assistance should be provided when a care recipient is in the toilet and change of parameters (such as a threshold for forward fall) in the falling down determination processing in the toilet. In this way, the same goes for the seating ability in that the ability presumption result based on sensor information at a certain location may affect processing at another location.

Meanwhile, as described previously, different patterns appear in the sensor information of the acceleration sensor 120 depending on the way of falling down during walking. For example, in a case where the walking ability of a care recipient is presumed based on one pattern, parameters (such as thresholds) used when the falling down determination processing based on another pattern is performed for this care recipient may be changed based on the walking ability thus presumed. For example, in a case where the processing capacity of the server system 300 has enough room to spare and where the number of ways of falling down of a care recipient increases for example, the falling down determination processing in which multiple patterns are combined may be performed. In this event, by reflecting the walking ability having been presumed on other patterns, it is possible to enhance processing precision.

Note that, in a case where there are multiple patterns by which the rhythm becomes off balance or where the number of patterns by which the rhythm becomes off balance increases than before, the method of this embodiment may recommend constant use of a foot pressure sensor to a nursing care staff in charge. This makes it possible to acquire detailed information on walking of a target care recipient and identify a pattern to be detected appropriately.

In addition, as described previously, the wearable module 100 may include a temperature sensor to detect a body surface temperature. For example, if determining that a care recipient has fallen down, the processing unit 310 activates the temperature sensor of the wearable module 100 corresponding to this care recipient and acquires a temperature change. By doing so, in a case where there is a possibility of injury such as bone fracture, it is possible to monitor vital information of the care recipient appropriately. Note that, by deactivating the temperature sensor except when falling down occurs, it is possible to reduce power consumption of the wearable module 100.

Meanwhile, in the falling down determination processing, the processing unit 310 may presume whether there is a possibility that a care recipient has hit his/her head by simulating the way of falling down. If determining that there is a possibility that the care recipient has hit his/her head, the processing unit 310 may present information on the necessity of detailed examination using the mobile terminal device 410 or the headset 420 of a caregiver.

### 3.4.3 Swallowing Ability

As described above using FIG. 25, in this embodiment, the swallowing time required for a care recipient to swallow food since he/she opens his/her mouth is measured based on the throat microphone TM and the camera of the communication device 200-5. The processing unit 310 may presume the swallowing ability of a care recipient based on a long-term change in the swallowing time. For example, the processing unit 310 continuously measures the swallowing time in the breakfast, lunch, dinner, snack, etc. in one day, and obtains the swallowing time of this day based on their average value and the like. Then, the processor determines a change of the values once data on the swallowing time per day for 30 days have been accumulated. For example, the processing unit 310 may determine the swallowing time on a per-month basis, and determine that the swallowing ability deteriorates if the swallowing time increases with time.

Further, besides the swallowing time, the processing unit 310 may classify the swallowing ability into multiple classes based on the swallowing sound e.g. the amplitude and cycle of a signal output from the throat microphone TM.

### 3.5 Application of Skeleton Tracking

Note that, the foregoing description has been given of the example of using the skeleton tracking for the bed position, the wheelchair position, and changing of a diaper, for example. However, the skeleton tracking may be used in other scenes.

For example, while a camera is disposed at a location where many people gather and do activities, such as a living room and hall of a nursing care facility, the skeleton tracking may be performed based on images taken by the camera. As described above using FIG. 2, while the communication device 200-6 is disposed on the TV set in the living room, for example, images may be taken using the camera of the communication device 200-6. In the example of FIG. 2, the communication device 200-6 outputs an image including three care recipients. For example, OpenPose described above discloses the method of performing the skeleton tracking for each of multiple persons taken in an image and displaying its result.

For example, the processing unit 310 may perform the skeleton tracking of each person in an image taken by the communication device 200-6 according to the same method, and perform processing for identifying a target care recipient by face recognition processing. Then, the processing unit 310 performs the falling down determination processing for each of care recipients based on the skeleton tracking result. For example, as described previously, the processing unit 310 may classify care recipients into classes according to their walking ability, seating ability, and the like and perform the falling down determination processing suitable for the class.

For example, a care recipient whose walking ability is low may fall down even by taking the standing posture. Accordingly, the processing unit 310 may determine whether the care recipient is taking the standing posture using the skeleton tracking. For example, if determining that the care recipient leans forward from the sitting posture with his/her hands placed on his/her knees, the seat surface of a chair, and the like, the processing unit 310 determines that the care recipient is taking the standing posture and notifies a caregiver of the risk of falling down.

Alternatively, while sectioning data to be processed into windows on a several-seconds basis, the processing unit 310 may determine that a posture change such as standing up occurs if the position of a specific portion such as the head or neck moves in each window by a predetermined threshold or more. Note that, the portion whose movement is to be detected may be other than the head and neck. In addition, the movement direction may be vertical, horizontal, or diagonal. Further, a threshold used for detection may be changed according to the portion to be detected. Furthermore, these conditions may be changed according to the attributes of the care recipient. Besides, various modifications are possible as the state of the care recipient and the risk of falling down that should be detected.

By doing so, even in a location where multiple care recipients do activities, it is possible to appropriately execute the falling down determination processing according to each care recipient.

### 4. End-of-Life Care

Meanwhile, implicit knowledge provided in this embodiment may include information giving suggestions for each of care recipients on whether end-of-life care should be started after a predetermined period. For example, the processing unit 310 acquires, as input data, five types of information including the amount or percentage of each type of food (e.g., may be for each of main and side dishes or may be for each of ingredients such as meat and fish) consumed at each meal, the amount of fluid intake, the timing when the meal is taken, information on diseases, and a weight (or BMI). Then, based on the input data, the processing unit 310 outputs output data indicating whether end-of-life care should be started after a predetermined period and whether it is the timing when the care contents should be changed after the end-of-life care is started. For example, machine learning may be performed based on training data in which ground truth data by a skilled worker is assigned to the input data. In this case, the processing unit 310 obtains output data by inputting the input data into learned model. Besides, other machine learning methods such as SVM may be used, or methods other than machine learning may be used.

End-of-life care mentioned here indicates assistance provided to a care recipient who is deemed to be highly likely to die in the near future. End-of-life care is different from normal assistance in that the emphasis is placed on alleviating physical and emotional pain, supporting a dignified life for a target care recipient, etc. In addition, since the condition of a care recipient changes with time during end-of-life care, assistance suitable for the target patient may change. In other words, by presenting the timing to start end-of-life care and the timing to change the assistance contents during the end-of-life care, it is possible to provide appropriate assistance to a care recipient to his/her last breath. For example, a skilled caregiver has implicit knowledge of presuming the timing when end-of-life care is needed and the care contents from various perspectives such as the volume of meal, and other caregivers can provide appropriate end-of-life care by digitizing such implicit knowledge.

FIGS. 32A to 32D illustrate an example of screens for displaying the end-of-life care determination result. The screens illustrated in FIGS. 32A to 32D may be displayed on the display of the mobile terminal device 410 or may be displayed on a display of a PC and the like used in a nursing care facility. Hereinbelow, a description will be given of an example where the mobile terminal device 410 is used.

FIG. 32A illustrates an example of a screen for uploading input data and giving instructions to execute analysis processing related to end-of-life care. Log data serving as input data of end-of-life care is stored on a per-care recipient basis in a management server of a nursing care facility and the storage unit of the mobile terminal device 410, for example. As described previously, the log data is time series data such as the amount or percentage of each type of food consumed at each meal, the amount of fluid intake, the timing when the meal is taken, information on diseases, and a weight or BMI. A user such as a caregiver presses an object OB12 which is a browse files button to designate a file which is the log data of a care recipient whom the user intends to set as an analysis target. In a box in FIG. 32A, the selected file name of the selected file is displayed, for example. When the user selects an object OB13 which is a start analysis button with the selected file designated, the mobile terminal device 410 or the like uploads the selected file to the server system 300. The processing unit 310 of the server system 300 obtains output data by inputting the selected file thus uploaded into learned model as input data. The processing unit 310 obtains the probability of starting end-of-life care after 30 days, for example. In addition, the processing unit 310 may output the transition prediction result of the amount of each type of food consumed at each meal or the like.

FIG. 32B illustrates an example of a screen for displaying an analysis result. FIG. 32B illustrates an example of the screen displayed when it is determined based on the output data that there is no need to start end-of-life care after 30 days. Note that, although FIG. 32B illustrates an example of using a file with extension .xlsx as an upload file, the data format is not limited to this. The same goes for FIG. 32C.

For example, the processing unit 310 of the server system 300 determines that there is no need to start end-of-life care if a probability value which is the output data is equal to or smaller than a given threshold. In this case, as illustrated in FIG. 32B, the display of the mobile terminal device 410 displays a text “there is no possibility of starting end-of-life care after 30 days” and an object including a check mark, for example.

FIG. 32C illustrates an example of a screen for displaying an analysis result, and illustrates an example of the screen displayed when there is a possibility of starting end-of-life care after 30 days. For example, the processing unit 310 of the server system 300 determines that there is a possibility of starting end-of-life care if a probability value which is the output data is larger than the given threshold described above. For example, the display of the mobile terminal device 410 displays a text “there is a possibility of starting end-of-life care”. As illustrated in FIG. 32C, the text may include the date of the input data and the date when there is a possibility of starting end-of-life care. In addition, as illustrated in FIG. 32, the display of the mobile terminal device 410 may display an object indicating a warning. Further, the display of the mobile terminal device 410 may display an object OB14 corresponding to a more details button for displaying the analysis result in more detail.

FIG. 32D illustrates an example of an analysis result screen displayed on the display of the mobile terminal device 410 when the object OB14 is selected. The analysis result screen may be displayed in a pop-up screen different from the screen of FIG. 32C, for example. However, their specific display aspects can be modified in various ways.

As illustrated in FIG. 32D, the analysis result screen may include a time series change in feature data obtained based on the input data and the result of determination on whether end-of-life care should be started after a predetermined period. The feature data mentioned here may be information, such as a moving average of the volume of meal, determined as important among the input information, or may be information obtained by calculation based on the five types of input information described above. For example, in the case of using the NN, the feature data may be an output from a given intermediate layer or output layer. For example, the input data includes the amount of main dish consumed, the amount of fluid, and a BMI actual measurement value acquired until Feb. 13, 2020. The processing unit 310 may presume the transition of the amount of main dish consumed, the amount of fluid, and BMI since Feb. 14, 2020 based on learned model. The analysis screen may include a graph representing a time series change of the actual measurement value and the presumed value for each of the three items. Note that, FIG. 32D illustrates a graph indicating a 7 days moving average of each of these values. This enables a caregiver to easily understand the transition of items important in end-of-life care. Note that, as described previously, the input data may include other items, and information to be displayed on the analysis result screen is not limited to that in the example of FIG. 32D.

Meanwhile, a period in which end-of-life care may be carried out may be displayed on the analysis result screen. In the example of FIG. 32D, a text “there is a possibility of end-of-life care from Mar. 14, 2020” is displayed, and the corresponding period in the graph is displayed so as to be identifiable by using a background color different from that of the other period. By doing so, the timing when and the period in which end-of-life care is presumed to be needed are specified clearly, so that information on end-of-life care can be presented to a user appropriately.

In the method of this embodiment, as described previously, the processing unit 310 may presume information after 30 days. For example, the processing unit 310 determines whether end-of-life care should be started after 30 days based on the input data. In this event, the data that serves as input may be configured in multiple ways. For example, the processing unit 310 may be capable of switching processing between processing of determining end-of-life care after 30 days based on input data such as the amount of food consumed for the past 15 days, and processing of determining end-of-life care after 30 days based on input data such as the amount of food consumed for the past 30 days.

Since end-of-life care is care provided right before a care recipient passes away, it may not be easy to collect a large volume of data used for determination. In that respect, by enabling determination with a relatively small volume of data such as the data for 15 days as described above, it is possible to make a determination on end-of-life care even in a phase where not enough data have been collected. Further, in a case where enough data have been collected, it is possible to improve determination precision by setting data for a relatively long period such as the data for 30 days as input data. Note that, although the two types of input data i.e. the data for 15 days and the data for 30 days are illustrated here, three or more types of input data target period may be provided. In addition, the timing to determine whether end-of-life care should be started is not limited to after 30 days. For example, the input data target period and the timing to determine whether end-of-life care should be started may be set by a user. For example, since the concept for end-of-life care is different from one facility to another, these values may be changed depending on the facility.

Meanwhile, in this embodiment, control to switch processing modes based on an output from a device may be performed on the basis of the result of determination on end-of-life care. FIG. 33 is a diagram illustrating the device related to the processing mode switching control. As illustrated in FIG. 33, the device mentioned here may be the sheet-shaped detection device 810 placed between the sections of the bed 510 and the mattress 820. The detection device 810 is configured to detect body vibration as a biological signal of a care recipient who is lying on the mattress 820. Then, the detection device 810 is configured to calculate biological information of the care recipient based on the vibration thus detected. For example, the biological information may include the respiratory rate, the heartbeat rate, and the amount of activity. Note that, the processing of obtaining biological information based on vibration is not limited to one executed by the detection device 810 and may be executed by the processing unit 310 of the server system 300, for example. Note that, such a detection device 810 is stated in Japanese Patent Application No. 2017-231224, filed on Nov. 30, 2017, and entitled “ABNORMALITY DETERMINATION DEVICE, PROGRAM”. This patent application is incorporated herein in its entirety by reference.

In Japanese Patent Application No. 2017-231224, it is determined whether a care recipient is close to the end of life based on the biological information. For example, a method is disclosed which determines whether a care recipient has such characteristics that he/she hardly moves or leaves the bed for a long period of time after the respiratory rate and heartbeat rate no longer show abnormal values, for example.

In the case of using it in combination with end-of-life care as in this embodiment, processing may be executed in such a way that processing in a normal mode is executed based on biological information output from the detection device 810 if it is determined that end-of-life care is not needed while processing in an abnormality determination mode is executed based on biological information output from the detection device 810 if it is determined that end-of-life care is needed. The normal mode is a processing mode without determination on the end of life, and may be a mode for determining a sleeping condition and the like based on the respiratory rate and heartbeat rate, for example. The abnormality determination mode is a mode for determining whether a care recipient is close to the end of life described above. Note that, the processing based on biological information which is an output from the detection device 810 may be executed by the server system 300, may be executed by the detection device 810, or may be executed by other devices such as the communication device 200. In other words, the processing mode mentioned here may represent the operation mode of the server system 300, may represent the operation mode of the detection device 810, or may represent the operation mode of other devices.

This makes it possible to use the result of determination on end-of-life care based on implicit knowledge and the processing mode based on biological information detected by the detection device 810 in conjunction with each other. Specifically, since when the end of life comes can be presumed roughly to some extent in end-of-life care, it is possible to execute processing in the abnormality determination mode when it is highly needed. In other words, processing in the normal mode is executed when it is determined that a care recipient is not close to the end of life, so that a decrease in processing load and the like are possible.

### 5. Recommendation

Meanwhile, in this embodiment, processing of recommending tools and instruments necessary for a care recipient may be performed based on the result of each determination processing having been described above.

For example, the processing unit 310 may recommend the type, size, etc. of a cushion to be used on the bed 510, the wheelchair 520, and the like based on information such as information on the bed position and the wheelchair position and information representing the attributes of a care recipient. In this event, the processing unit 310 may make recommendation using information that has been collected in a facility different from a facility where a care recipient to be determined is living. In addition, the processing unit 310 may recommend the type of a diaper and the type of a pad based on information that has been collected at the time of using implicit knowledge in changing a diaper. Further, the processing unit 310 may recommend a change of the type of tools, such as a spoon and a self-help device used in taking a meal, based on information that has been collected at the time of using implicit knowledge in taking a meal.

Meanwhile, the processing unit 310 may recommend a tilting wheelchair or a reclining wheelchair according to the presumed seating ability and walking ability. More specifically, the processing unit 310 may presume the timing to repurchase a wheelchair or a necessary rental period of a wheelchair through machine learning with time series data on the seating ability set as input data. This makes it possible to create an efficient plan for using a device having a high unit cost. In addition, the processing unit 310 may predict how much the need of nursing care level becomes higher through machine learning with time series data on the seating ability and walking ability set as input data, and recommend a nursing care item according to the prediction result. For example, assuming an example where the need of nursing care level becomes higher in the order of independent walking, walking using a stick, wheeled walker, and the processing unit 310 may recommend the timing to purchase a stick or wheeled walker and the type of a stick etc. recommended for use.

Further, the processing unit 310 may make comprehensive recommendation of instruments considered necessary for a target care recipient, such as a walking aid, a wheelchair, a bed, etc., upon accepting input on several items, such as living environment/equipment environment/space at home and the facility, the concept of the facility and family, etc. The concept of the facility and family is information that represents the family's or facility personnel's thoughts on what style of living they would like a care recipient to have, for example, to make use of residual abilities while ensuring safety. This makes it possible to collectively propose instruments necessary for the family, facility, etc., and thus possible to increase the level of convenience for a caregiver.

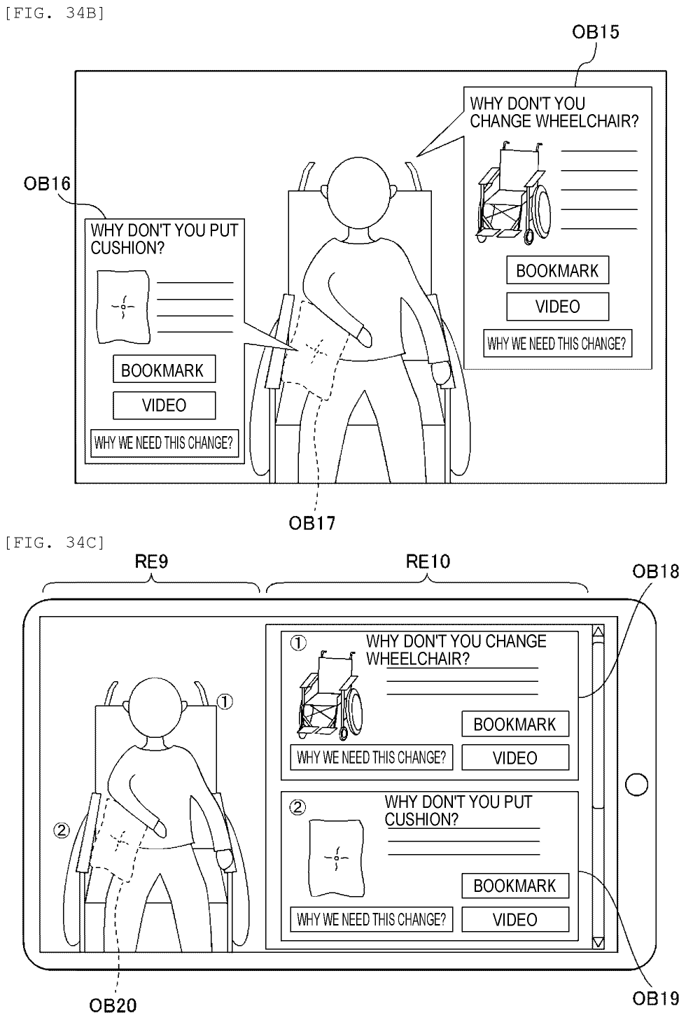

FIG. 34A illustrates an example of a system used for recommendation. For example, a caregiver wears, as the caregiver terminal 400, an eyeglasses-type device 430 which is an AR glass or an MR glass. The eyeglasses-type device 430 has a camera that takes an image of a region corresponding to a user's field of view, for example. The eyeglasses-type device 430 has a lens portion a part or all of which serves as a display, and enables a user to visually check the situation of the surrounding by transmitting light from the surrounding through the display or by displaying an image corresponding to the user's field of view that is taken by the camera. Further, using the display, the eyeglasses-type device 430 additionally displays some sort of information on the user's field of view. For example, as illustrated in FIG. 34A, in response to an event where a caregiver views a care recipient while wearing the eyeglasses-type device 430, recommendation suitable for this care recipient is displayed on the display of the eyeglasses-type device 430. For example, control to display a recommendation screen may be performed upon detection of a care recipient, for whom recommendation is to be made, as a result of face recognition processing of the care recipient in a processing unit of the eyeglasses-type device 430 or the processing unit 310 of the server system 300.

FIG. 34B illustrates an example of a recommendation display screen. As illustrated in FIG. 34B, an image taken by the eyeglasses-type device 430 includes a target care recipient and the wheelchair 520, for example. In this case, instruments etc. recommended when the target care recipient moves with the wheelchair 520 may be recommended. For example, the processing unit 310 recognizes assistance instruments and the like located near the care recipient in addition to face recognition processing of the care recipient, and displays recommendation information based on this result. Note that, information indicating which of the communication devices 200 the wearable module 100 is connected to may be used for identifying the instruments located near the care recipient.

In the example of FIG. 34B, the display of the eyeglasses-type device 430 displays, on the image in which the care recipient is taken, an object OB15 indicating recommendation information on a new wheelchair and an object OB16 indicating recommendation information on a cushion. As illustrated in FIG. 34B, the object OB15 displays a text “why don't you change a wheelchair?”. In addition, the fact that this object corresponds to the wheelchair 520 in the taken image is specified clearly using a dialogue balloon frame. This makes it possible to deliver, to a caregiver etc., an easy-to-understand message that proposes replacing the wheelchair 520 being used with a new wheelchair.

For example, the object OB15 includes information such as an image of an instrument to be proposed, a text explaining its features, its price, and an evaluation value made by a user who uses it. The object OB15 may also include a bookmark button, a video button, and a reason display button. The bookmark button is a button for enabling a caregiver to easily access information on the instrument displayed. For example, in the case of selecting the bookmark button on the screen illustrated in FIG. 34B, information on the wheelchair displayed is stored as a bookmark in association with the caregiver. For example, in a case where the caregiver selects the bookmark using the caregiver terminal 400, information which is the same as the object OB15 or information corresponding to the object OB15 is presented to the caregiver terminal 400. For example, an image, price, etc. included in the object OB15 are information extracted from a website of a manufacturer and a shopping website, and the bookmark may be information indicating their URLS.

Meanwhile, the video button is a button for displaying a video related to a target instrument. The video mentioned here may be a promotion video created by an instrument manufacturer or may be a review video posted by a user who uses this instrument. In addition, other application software such as video posting/browsing application may be activated when the video button is pressed. For example, a search result screen obtained by searching for a video by a product name may be displayed in response to an event where the video button is pressed.

The reason display button is a button for displaying the reason why the target instrument is recommended. As described previously, in this embodiment, the falling down determination processing and the determination on the seating ability are performed, and other determinations using implicit knowledge are also performed in various scenes, and instruments etc. to be recommended are determined as a result. By presenting the reason of determination based on the reason display button, it is possible to present information for a caregiver, a care recipient, the family of the care recipient, or the like to make a determination on whether to introduce the target instrument.

The object OB16 is an example of recommendation information for recommending a cushion. Since information to be displayed is the same as that of the object OB15, its detailed description will be omitted. Note that, an object OB17 indicating a location where the target cushion should be disposed may be displayed in conjunction with the object OB16. In the example of FIG. 34B, the object OB17 is displayed on the right side of a care recipient. This makes it possible to recommend not only the name and type of a product but also where it should be disposed and how to use it. For example, the cushion illustrated in FIG. 34B may be recommended if it is determined that the care recipient suffers paralysis on one side of his/her body based on the recognition result of the taken image or information such as the attributes of the care recipient. This makes it possible to present, to a caregiver etc., a cushion usable for preventing contracture etc. and how to use this cushion.

Meanwhile, although the foregoing description has been given of the example of displaying the recommendation information using the eyeglasses-type device 430, the display method is not limited to this. For example, the recommendation information may be displayed in the same manner using an AR app in a smartphone etc. FIG. 34C illustrates an example of a screen displayed on a display of a smartphone. In a region RE9 of FIG. 34C, an image obtained by superimposing numbers and an object OB20 on an image taken by a camera of the smartphone is displayed. In addition, in a region RE10, objects OB18 and OB19 indicating recommendation information are each displayed in association with the same number in the region RE9. Note that, since the objects OB18 to OB20 are the same as the objects OB15 to OB17 in FIG. 34B, their detailed description will be omitted.

This makes it possible to browse recommendation information using a device widely used such as a smartphone. For example, the family of a care recipient etc. can browse the screen of FIG. 34C by taking an image of the care recipient using his/her own smartphone, and thus can acquire recommendation information easily.

Note that, although this embodiment has been described in detail above, it will be readily understood by those skilled in the art that various modifications are possible that do not materially depart from the new matters and effects of this embodiment. Accordingly, all of these modifications shall fall within the scope of this disclosure. For example, the term that is mentioned at least once in the specification or drawings with a different term that is a broader term or synonym may be replaced by that different term at any point in the specification or drawings. In addition, all combinations of this embodiment and the modifications shall fall within the scope of this disclosure. Further, the configuration, operation, and the like of the wearable module, the communication device, the server system, etc. are not limited to those described in this embodiment and various modifications are possible.

1.-12. (canceled)

13. An information processing apparatus comprising:
a receiver configured to acquire sensor information received from a wearable device and location information identifying a location of a communication device which receives the sensor information; and
a controller configured to execute, based on the location information and the sensor information, an evaluation processing to evaluate a risk of a user who wears the wearable device, wherein the evaluation processing being configured to be changed based on the location information.

14. The information processing apparatus according to claim 13, wherein
the location of the communication device includes a location of a bed, a location of a wheelchair, and a location of a toilet, and
the controller is configured to execute the evaluation processing to determine whether the user may be falling down, and
wherein the evaluation processing to determine whether the user may be falling down from the bed, the evaluation processing to determine whether the user may be falling down from the wheelchair, the evaluation processing to determine whether the user may be falling down in the toilet, and the evaluation processing to determine whether the user may be falling down during walking are different respectively.

15. The information processing apparatus according to claim 13, wherein the controller is configured to:
execute a processing identifying a peripheral device located around the user based on at least one of the location information and information identifying the user who wears the wearable device, and
control the peripheral device based on the evaluation processing to determine whether the user may be falling down.

16. The information processing apparatus according to claim 15, wherein
the peripheral device is a device including a caster, and
the controller is configured to lock the caster of the peripheral device if the controller determines the user has the risk of falling down.

17. The information processing apparatus according to claim 15, wherein
the peripheral device is a device including a caster, and
if the controller determines the user has the risk of falling down, the controller is configured to control the peripheral device to move the peripheral device closer to the user by driving the caster of the peripheral device.

18. The information processing apparatus according to claim 17, wherein the peripheral device including the caster includes at least one of a table and a wheeled walker.

19. The information processing apparatus according to claim 14, wherein the controller is configured to:
presume a seating ability, which represents an ability of the user to keep a seated position, based on any of the sensor information corresponding to the bed and the sensor information corresponding to the wheelchair, and
based on the presumed seating ability, execute a processing whether the user needs assistance at other locations including at least the toilet.

20. The information processing apparatus according to claim 14, wherein the controller is configured to:
presume a walking ability, which represents an ability of the user to walk stably, based on the sensor information corresponding to the during walking, and
based on the presumed walking ability, execute a processing whether the user needs assistance at other locations including at least the toilet.

21. The information processing apparatus according to claim 13, wherein a controller is configured to:
execute a processing identifying a peripheral device located around the user based on the location information,
activate the peripheral device,
execute, based on the sensor information from the peripheral device, an evaluation processing to evaluate the risk of a user who wears the wearable device.

22. The information processing apparatus according to claim 13, wherein a controller is configured to:
activate a first device including a throat microphone can be mounted on a neck of the user and a camera if the controller identifies the first device as the peripheral device located around the user,
execute, based on the location information and the sensor information from the first device, the evaluation processing to evaluate the aspiration risk of the user, the sensor information from the first device including information whether the user is choking or swallowing, and information whether a mouth of the user is open or closed.

23. The information processing apparatus according to claim 13, wherein a controller is configured to:
activate a second device including a camera and a display if the controller identifies the second device as the peripheral device located around the user,
execute, based on the location information and the sensor information from the second device, the evaluation processing to evaluate the bed sore risk of the user, the sensor information from the second device including information representing a posture of the user.

24. The information processing apparatus according to claim 22, wherein a controller is configured to:
activate a second device including a camera and a display if the controller identifies the second device as the peripheral device located around the user,
execute, based on the location information and the sensor information from the second device, the evaluation processing to evaluate the bed sore risk of the user, the sensor information from the second device including information representing a posture of the user, the evaluation processing to evaluate the bed sore risk of the user being different from the evaluation processing to evaluate the aspiration risk of the user.

25. The information processing apparatus according to claim 23, wherein the second device is configured to display an image in real time while superimposing a correct image having been subjected to transparent processing on the image in real time.

26. The information processing apparatus according to claim 25, wherein the second device is configured to:
determine a posture of the user is correct or not based on the image in real time and the correct image, and
display a result indicating whether the posture of the user is correct or not.

27. The information processing apparatus according to claim 26, wherein the second device is configured to display recommend information based on the result indicating whether the posture of the user is correct or not.

28. The information processing apparatus according to claim 13, wherein a controller is configured to:
activate a third device including pressure sensors which can be installed on the wheelchair if the controller identifies the third device as the peripheral device located around the user,
execute, based on the location information and the sensor information from the third device, the evaluation processing to evaluate whether a forward displacement or a lateral displacement occurs, the sensor information from the third device including pressure information on the wheelchair.

29. An information processing method comprising the steps of:
acquiring sensor information received from a wearable device and location information identifying a location of a communication device which receives the sensor information;
executing, based on the location information and the sensor information, an evaluation processing to evaluate a risk of a user who wears the wearable device, wherein the evaluation processing being configured to be changed based on the location information.
